(19) United States
(12) Patent Application Publication
(10) Pub. No.: US 2018/0011098 A1
(43) Pub. Date: Jan. 11, 2018

(54) METHODS AND KITS FOR ANALYSIS OF HMGB1 ISOFORMS

(71) Applicant: University of Hawaii, Honolulu, HI (US)

(72) Inventors: Haining Yang, Honolulu, HI (US); Michele Carbone, Honolulu, HI (US)

(73) Assignee: University of Hawaii, Honolulu, HI (US)

(21) Appl. No.: 15/544,835

(22) PCT Filed: Jan. 19, 2016
(86) PCT No.: PCT/US2016/013964

§ 371 Date: Jul. 19, 2017

Related U.S. Application Data
(60) Provisional application No. 62/106,092, filed on Jan. 21, 2015.

(51) Int. Cl. G01N 33/574; C07K 16/24; G01N 33/68

(52) U.S. Cl. CPC G01N 33/57423; G01N 33/6863; G01N 33/57415; G01N 33/57419; G01N 33/57438; C07K 16/24; G01N 33/57488; G01N 2333/52; G01N 2440/10; G01N 2440/20; G01N 2800/40

(57) ABSTRACT

In accordance with some embodiments herein, methods of determining signatures of HMGB1 isoforms in a subject are provided. In some embodiments, antibodies that bind specifically to HMGB1 isoforms are provided. In some embodiments, immunoassay kits are provided.

### CROSS-REFERENCE TO RELATED APPLICATIONS

This application claims the benefit of U.S. Provisional Application No. 62/106,092 filed Jan. 21, 2015, which is hereby incorporated by reference in its entirety.

### REFERENCE TO SEQUENCE LISTING

The present application is being filed along with a Sequence Listing in electronic format. The Sequence Listing is provided as a file entitled UOH051WOSEQUENCE.TXT, created and last saved on Jan. 18, 2016, which is 2,198 bytes in size. The information in the electronic format of the Sequence Listing is incorporated herein by reference in its entirety.

### FIELD

Some embodiments herein relate generally to the field of cancer biology, pathology, and diagnostics. Some embodiments relate to detecting asbestos exposure or malignant mesothelioma or inflammatory cancer. Some embodiments relate to differentiating whether a subject has malignant mesothelioma or asbestos exposure.

### BACKGROUND

Malignant mesothelioma (MM) is a relatively rare, aggressive tumor with prognosis of about 1 year, usually associated to chronic exposure to carcinogenic mineral fibers such as asbestos and erionite. In the US alone, 27 million people have been exposed to asbestos fibers, and MM causes about 3,200 deaths per year. Asbestos refers to a family of mineral fibers that includes crocidolite, often considered the most oncogenic type. Since asbestos does not induce malignant transformation of primary human mesothelial cells (HM) directly, indirect mechanisms of carcinogenesis have been investigated. Inhaled asbestos fibers become entrapped in the lung and some migrate through the lymphatics to the pleura.

A number of other inflammatory cancers are known, for example mesothelioma, colorectal cancer, pancreatic cancer, breast cancer, and liver cancer.

High Mobility Group Box 1 protein (HMGB1) is a prototypical damage-associated molecular pattern molecule (DAMP).

### SUMMARY

In some embodiments, a method of identifying asbestos exposure, malignant mesothelioma, or inflammatory cancer in a subject is provided. The method can comprise determining an amount of an isoform of HMGB1 in a biological sample of the subject, wherein the isoform of HMGB1 is selected from (a) a 24.585 KDa isoform of HMGB1; (b) a 24.587 KDa isoform of HMGB1; (c) a 25.467 KDa isoform of HMGB1; or (d) a 25.469 KDa isoform of HMGB1. The method can comprise comparing an amount of the isoform of HMGB1 to a predetermined level, wherein an amount greater than the predetermined level identifies asbestos exposure, malignant mesothelioma, or inflammatory cancer in the subject. In some embodiments, (a) a hypo-acetylated disulfide isoform of HMGB1 has a mass of about 24.585 kDa; (b) a hypo-acetylated fully-reduced isoform of HMGB1 has a mass of about 24.587 kDa; (c) a hyper acetylated disulfide isoform of HMGB1 has a mass of about 25.467 kDa; and/or (d) a hyper-acetylated fully-reduced isoform of HMGB1 has a mass of about 25.693 kDa. In some embodiments, a level of (c) or (d) greater than 5 ng/mL indicates a presence of mesothelioma, and a level of (a) or (d) greater than 5-fold of a lower limit of detection indicates a presence of mesothelioma or inflammatory cancer. In some embodiments, a level of (c) or (d) less than 1 ng/mL and a level of (a) or (d) greater than 2 ng/mL indicates asbestos exposure, but not mesothelioma. In some embodiments, the inflammatory cancer is selected from the group consisting of: mesothelioma, colorectal cancer, pancreatic cancer, breast cancer, and liver cancer. In some embodiments, the amount of the isoform of HMGB1 in the biological sample is determined by mass spectrometry. In some embodiments, the amount of the isoform of HMGB1 in the biological sample is determined by contacting the sample with: (a) an antibody that binds specifically to hypo-acetylated disulfide HMGB1 to detect the 24.585 kDa isoform; (b) an antibody that binds specifically to hypo-acetylated fully reduced HMGB1 to detect the 24.587 kDa isoform; (c) an antibody that binds specifically to hyper-acetylated disulfide HMGB1 to detect the 25.467 isoform; or (d) an antibody that binds specifically to hyper-acetylated fully-reduced HMGB1 to detect the 25.693 kDa isoform. In some embodiments, malignant mesothelioma patients are discriminated from patients with pleural effusions (malignant or benign), for example based on total HMGB1 levels, or hyper-acetylated HMGB1 levels.

In some embodiments, a method of identifying asbestos exposure, malignant mesothelioma, or inflammatory cancer in a subject is provided. The method can comprise determining an amount of an isoform of HMGB1 in a biological sample of the subject, wherein the isoform of HMGB1 is selected from (a) a hypo-acetylated disulfide isoform of HMGB1; (b) a hypo-acetylated fully-reduced isoform of HMGB1; (c) a hyper acetylated disulfide isoform of HMGB1; or (d) a hyper-acetylated fully-reduced isoform of HMGB1. The method can comprise comparing an amount of the isoform of HMGB1 to a predetermined level, wherein an amount greater than the predetermined level identifies asbestos exposure, malignant mesothelioma, or inflammatory cancer in the subject. In some embodiments, (a) the hypo-acetylated disulfide isoform of HMGB1 has a mass of about 24.585 kDa; (b) the hypo-acetylated fully-reduced isoform of HMGB1 has a mass of about 24.587 kDa; (c) the hyper acetylated disulfide isoform of HMGB1 has a mass of about 25.467 kDa; and/or (d) the hyper-acetylated fully-reduced isoform of HMGB1 has a mass of about 25.693 kDa. In some embodiments, a level of (c) or (d) greater than 5 ng/mL indicates a presence of mesothelioma, and a level of (a) or (d) greater than 5-fold of a lower limit of detection indicates a presence of mesothelioma or inflammatory cancer. In some embodiments, a level of (c) or (d) less than 1 ng/mL and a level of (a) or (d) greater than 2 ng/mL indicates asbestos exposure, but not mesothelioma. In some embodiments, the inflammatory cancer is selected from the group consisting of: mesothelioma, colorectal cancer, pancreatic cancer, breast cancer, and liver cancer. In some embodiments, the amount of the isoform of HMGB1 in the biological sample is determined by mass spectrometry. In some embodiments, the amount of the isoform of HMGB1 in the biological sample is determined by contacting the sample with: (a) an antibody that binds specifically to hypo-acetylated disulfide HMGB1 to detect the 24.585 kDa isoform; (b) an antibody that binds specifically to hypo-acetylated fully reduced HMGB1 to detect the 24.587 kDa isoform; (c) an antibody that binds specifically to hyper-acetylated disulfide HMGB1 to detect the 25.467 isoform; or (d) an antibody that binds specifically to hyper-acetylated fully-reduced HMGB1 to detect the 25.693 kDa isoform. In some embodiments, malignant mesothelioma patients are discriminated from patients with pleural effusions (malignant or benign), for example based on total HMGB1 levels, or hyper-acetylated HMGB1 levels.

In some embodiments, a method of detecting an HMGB1 isoform signature in a sample of a subject is provided. The method can comprise providing a sample of a subject, for example a biological sample. The method can comprise contacting the sample with at least one of: (a) an antibody that binds specifically to hyper-acetylated HMGB1; (b) an antibody that binds specifically to disulfide HMGB1 (HMGB1C23-C45); (c) an antibody that binds specifically to hypo-acetylated HMGB1; (d) an antibody that binds specifically to fully reduced HMGB1; (e) an antibody that binds specifically to hyper-acetylated disulfide HMGB1; (f) an antibody that binds specifically to hyper-acetylated fully reduced HMGB1; (g) an antibody that binds specifically to hypo-acetylated disulfide HMGB1; or (h) an antibody that binds specifically to hypo-acetylated fully-reduced HMGB1. The method can comprise detecting a level of the antibody bound to HMGB1 in the sample. In some embodiments, the method is performed in vitro. In some embodiments, the method further comprises comparing the level of bound antibody to a predetermined level. In some embodiments, binding of any of (a)-(h) below the predetermined level identifies an HMGB1 isoform signature characteristic of a healthy subject. In some embodiments, binding of any of (a)-(h) above the predetermined level identifies an HMGB1 isoform characteristic of a subject that has at least one of asbestos exposure, malignant mesothelioma or an inflammatory cancer. In some embodiments, the sample is contacted with (a) and (c), and wherein binding of (c) above a first predetermined level and binding of (a) above a second predetermined level identifies an HMGB1 isoform characteristic of a subject that has malignant mesothelioma or an inflammatory cancer. In some embodiments, the sample is contacted with (a) and (c), and wherein binding of (c) above a first predetermined level and binding of (a) below a second predetermined level identifies an HMGB1 isoform characteristic of a subject that has asbestos exposure, but not malignant mesothelioma. In some embodiments, the sample is contacted with (b) and (d), and wherein binding of (d) above a first predetermined level and binding of (b) above a second predetermined level identifies an HMGB1 isoform characteristic of a subject that has malignant mesothelioma or an inflammatory cancer. In some embodiments, the sample is contacted with (b) and (d), and wherein binding of (d) above a first predetermined level and binding of (b) below a second predetermined level identifies an HMGB1 isoform characteristic of a subject that has asbestos exposure, but not malignant mesothelioma. In some embodiments, the sample is contacted with (a) and (c), and wherein binding of (c) above a first predetermined level and binding of (a) above a second predetermined level identifies an HMGB1 isoform characteristic of a subject that has an inflammatory cancer. In some embodiments, the sample is contacted with (b) and (d), and wherein binding of (d) above a first predetermined level and binding of (b) above a second predetermined level identifies an HMGB1 isoform characteristic of a subject that has an inflammatory cancer. In some embodiments, binding of any of (a)-(h) above the predetermined level identifies an HMGB1 isoform characteristic of a subject that has an inflammatory cancer. In some embodiments, the inflammatory cancer is selected from the group consisting of mesothelioma, colorectal cancer, pancreatic cancer, breast cancer, and liver cancer. In some embodiments, the antibody that binds specifically to hyper-acetylated HMGB1 binds specifically to an acetylated lysine in NLS1 or an acetylated lysine in NLS2 of HMGB1. In some embodiments, the antibody that binds specifically to hyper-acetylated HMGB1 binds specifically to one of acetylated K30, acetylated K43, acetylated K90, or acetylated K141 of HMGB1. In some embodiments, the antibody that binds specifically to disulfide HMGB1 binds specifically to a C23 and/C45 of HMGB1 when C23 and C45 of HMGB1 are joined by a disulfide bond. In some embodiments, the antibody that binds specifically to fully reduced HMGB1 binds specifically to at least one of reduced C23, reduced C45, or reduced C106 of HMGB1. In some embodiments, the sample comprises a plasma or serum sample. In some embodiments, the sample comprises a serum sample. In some embodiments, the sample comprises a plasma sample. In some embodiments, the method further comprises comparing binding of the antibody to a negative control. In some embodiments, the negative control comprises a sample of an individual known not to have mesothelioma. In some embodiments, the method further comprises contacting the sample with an antibody that binds specifically to HMGB1 of any isoform, determining the level of total HMGB1 in the sample, and comparing the level of total HMGB1 to a negative control or predetermined level. In some embodiments, if the subject is a smoker, the negative control is a smoker, and wherein if the subject comprises a non-smoker, the negative control comprises a non-smoker. In some embodiments, if the subject is a smoker, the predetermined level is higher than if the subject is a non-smoker. In some embodiments, a subject having malignant mesothelioma is discriminated from a subject with pleural effusions (malignant and/or benign), for example based on total HMGB1 levels, or hyper-acetylated HMGB1 levels. In some embodiments, binding of any of (a)-(h) below the predetermined level identifies an HMGB1 isoform signature characteristic of a subject who does not have malignant mesothelioma. In some embodiments, binding of any of (a)-(h) below the predetermined level identifies an HMGB1 isoform signature characteristic of a subject who does not have malignant mesothelioma, but without ruling-out pleural effusions (malignant and/or benign).

In some embodiments, a method of identifying malignant mesothelioma in a subject is provided. The method can comprise providing a sample of the subject, for example a biological sample. The method can comprise contacting the sample with at least one of (a) an antibody that binds specifically to hyper-acetylated HMGB1; (b) an antibody that binds specifically to disulfide HMGB1 (HMGB1C23-C45); (c) an antibody that binds specifically to hypo-acetylated HMGB1; or (d) an antibody that binds specifically to fully reduced HMGB1. The method can comprise detecting a level of the antibody bound to HMGB1 in the sample. The method can comprise comparing the level of binding of antibody to a predetermined level. The method can comprise diagnosing the subject as having malignant mesothelioma when the level of antibody bound to HMGB1 is greater than the predetermined level. In some embodiments, the sample is contacted with (a), and the predetermined level is a level of the antibody of (a) bound to 1 ng/mL hyper-acetylated HMGB1. In some embodiments, the sample is contacted with (a), and the predetermined level is a level of the antibody of (a) bound to 2 ng/mL hyper-acetylated HMGB1. In some embodiments, the predetermined level is a level of the antibody of (a) bound to at least 2 ng/mL hyper-acetylated HMGB1, for example 2 ng/ml, 3, 4, 5, 6, 7, 8, 9, 10, 15, or 20 ng/mL hyper-acetylated HMGB1. In some embodiments, the sample is contacted with (a), and the predetermined level is a level of the antibody of (a) bound to 5 ng/mL hyper-acetylated HMGB1. In some embodiments, the sample is contacted with (b), and the predetermined level is a level of the antibody of (b) bound to 0.1 ng/mL disulfide HMGB1. In some embodiments, the predetermined level is a level of the antibody of (b) bound to at least 0.2 ng/mL disulfide HMGB1, for example 0.2 ng/mL, 0.3, 0.4, 0.5, 0.9, 1, 1.5, 2, 3, 4, 5, 6, 7, 8, 9, 10, 15, or 20 ng/mL disulfide HMGB1. In some embodiments, the sample is contacted with (b), and the predetermined level is a lower limit of detection of the antibody of (b) bound to disulfide HMGB1. In some embodiments, the sample is contacted with (b), and the predetermined level is a level of the antibody of (b) bound to 5 ng/mL disulfide HMGB1. In some embodiments, the sample is contacted with at least two of (a), (b), (c), and (d). In some embodiments, the sample is contacted with (a), and the predetermined level is a level of the antibody of (a) bound to 1 ng/mL hyper-acetylated HMGB1, and the sample is further contacted with (b), (c), or (d). In some embodiments, the sample is contacted with (a), and the predetermined level is a level of the antibody of (a) bound to 2 ng/mL hyper-acetylated HMGB1, and the sample is further contacted with (b), (c), or (d). In some embodiments, the predetermined level is a level of the antibody of (a) bound to at least 1 ng/mL hyper-acetylated HMGB1, for example 1 ng/ml, 2, 3, 4, 5, 6, 7, 8, 9, 10, 15, 20, 25, 30, 35, 40, 45, or 50 ng/mL hyper-acetylated HMGB1. In some embodiments, the sample is contacted with (b), and the predetermined level is a lower limit of detection of the antibody of (b) bound to disulfide HMGB1, and the sample is further contacted with (a), (c), or (d). In some embodiments, the sample is contacted with (b), and the predetermined level is a level of the antibody of (b) bound to 0.1 ng/ml disulfide HMGB1, and the sample is further contacted with (a), (c), or (d). In some embodiments, the predetermined level is a level of the antibody of (b) bound to at least 0.2 ng/mL disulfide HMGB1, for example 0.2 ng/mL, 0.3, 0.4, 0.5, 0.9, 1, 1.5, 2, 3, 4, 5, 6, 7, 8, 9, 10, 15, or 20 ng/mL disulfide HMGB1. In some embodiments, the sample of the subject comprises a fluid. In some embodiments, the sample of the subject comprises a serum or a plasma. In some embodiments, the sample of the subject comprises a serum. In some embodiments, the sample of the subject comprises a plasma. In some embodiments, the antibody that binds specifically to hyper-acetylated HMGB1 binds specifically to an acetylated lysine in NLS1 or an acetylated lysine in NLS2 of HMGB1. In some embodiments, the antibody that binds specifically to hyper-acetylated HMGB1 binds specifically to one of acetylated K30, acetylated K43, acetylated K90, or acetylated K141 of HMGB1. In some embodiments, the antibody that binds specifically to disulfide HMGB1 binds specifically to a C23 and/C45 of HMGB1 when C23 and C45 of HMGB1 are joined by a disulfide bond. In some embodiments, the antibody that binds specifically to fully reduced HMGB1 binds specifically to at least one of reduced C23, reduced C45, or reduced C106 of HMGB1. In some embodiments, the antibody further comprises a detectable moiety. In some embodiments, the method comprises an in vitro method. In some embodiments, the method comprises an in vivo method. In some embodiments, the detection of a presence of disulfide or hyper-acetylated HMGB1 indicates that the subject has malignant mesothelioma. In some embodiments, the detection of a presence of disulfide HMGB1 indicates that the subject has malignant mesothelioma. In some embodiments, the detection of a presence of hyper-acetylated HMGB1 indicates that the subject has malignant mesothelioma. In some embodiments, the method further comprises diagnosing the subject as not having malignant mesothelioma, when the level of antibody bound to HMGB1 is less than the predetermined level. In some embodiments, the detection of a presence of disulfide or hyperacetylated HMGB1 indicates that the subject has malignant mesothelioma. In some embodiments, the method further comprises diagnosing the subject as not having malignant mesothelioma, though not ruling-out pleural effusions, when the level of antibody bound to HMGB1 is less than the predetermined level.

In some embodiments, a method of differentiating between malignant mesothelioma and asbestos-exposure in a subject is provided. The method can comprise providing a sample from a subject suspected of having malignant mesothelioma, for example a biological sample. The method can comprise contacting the sample with at least one of: (a) an antibody that binds specifically to hyper-acetylated HMGB1; (b) an antibody that binds specifically to disulfide HMGB1 (HMGB1C23-C45); or (c) an antibody that binds specifically to fully reduced HMGB1. The detecting a level of the antibody bound to hyper-acetylated HMGB1, disulfide HMGB1 (HMGB1C23-C45) or fully reduced HMGB1; and comparing the level of antibody bound to hyper-acetylated HMGB1, disulfide HMGB1, or fully reduced HMGB1 to a predetermined level, wherein a level of hyper-acetylated HMGB1, disulfide HMGB1, or fully reduced HMGB1 greater than a predetermined level indicates a presence of malignant mesothelioma in the subject. In some embodiments, the sample is contacted with (a), and wherein the predetermined level is a level of the antibody of (a) bound to 2 ng/mL of hyper-acetylated HMGB1. In some embodiments, the predetermined level is a level of the antibody of (a) bound to at least 1 ng/mL hyper-acetylated HMGB1, for example 1 ng/ml, 2, 3, 4, 5, 6, 7, 8, 9, 10, 15, or 20 ng/mL hyper-acetylated HMGB1. In some embodiments, the sample is contacted with (a), and wherein the predetermined level is a level of the antibody of (a) bound to 5 ng/mL of hyper-acetylated HMGB1. In some embodiments, the sample is contacted with (b), and wherein the predetermined level is a lower limit detection of the antibody of (b). In some embodiments, the sample is contacted with (b), and wherein the predetermined level is a 5-fold increase over a lower limit detection of the antibody of (b). In some embodiments, the sample is contacted with (b), and wherein the predetermined level is a level of the antibody of (b) bound to 0.1 ng/mL of disulfide HMGB1. In some embodiments, the predetermined level is a level of the antibody of (b) bound to at least 0.1 ng/mL disulfide HMGB1, for example 0.1 ng/ml, 0.2, 0.3, 0.4, 0.5, 0.6, 0.7, 0.8, 0.9, 1, 1.5, 2, 3, 4, 5, 6, 7, 8, 9 or 10 ng/mL disulfide HMGB1. In some embodiments, the detection of a presence of disulfide HMGB1 indicates that the subject has malignant mesothelioma. In some embodiments, the antibody that binds specifically to hyper-acetylated HMGB1 binds specifically to an acetylated lysine in NLS1 or an acetylated lysine in NLS2 of HMGB1. In some embodiments, the antibody that binds specifically to hyper-acetylated HMGB1 binds specifically to one of acetylated K30, acetylated K43, acetylated K90, or acetylated K141 of HMGB1. In some embodiments, the antibody that binds specifically to disulfide HMGB1 binds specifically to a C23 and/C45 of HMGB1 when C23 and C45 of HMGB1 are joined by a disulfide bond. In some embodiments, the antibody that binds specifically to fully reduced HMGB1 binds specifically to at least one of reduced C23, reduced C45, or reduced C106 of HMGB1. In some embodiments, the method comprises an in vitro method. In some embodiments, the method comprises an in vivo method. In some embodiments, the antibody comprises a detectable moiety.

In some embodiments, any of the above methods further comprises contacting the sample with an antibody that binds specifically to total HMGB1. In some embodiments, for any of the above methods, the predetermined level is selected based on at least one of sex, age, or smoking status of the subject. In some embodiments, for any of the above methods, the predetermined level is higher for a female subject than for a male subject. In some embodiments, for any of the above methods, the predetermined level is higher for a subject 55 years or older than for a subject under the age of 55 years. In some embodiments, for any of the above methods the predetermined level is higher for a subject who is a previous or active smoker than for a subject who is a non-smoker. In some embodiments, for any of the above methods, the antibody comprises a non-native antibody. In some embodiments, for any of the above methods, the antibody comprises a monoclonal antibody. In some embodiments, for any of the above methods, the antibody comprises an antibody engineered against an HMGB1 isoform. In some embodiments, any of the above methods further comprises detecting a binding pattern of the antibody. In some embodiments, for any of the above methods, the predetermined level is based upon an electronically stored or written value. In some embodiments, for any of the above methods the predetermined level is based upon a control or standard sample. In some embodiments, any of the above methods further comprises recommending a treatment regimen for malignant mesothelioma or inflammatory cancer for a subject for whom the level of bound antibody exceeds the predetermined level. In some embodiments, for any of the above methods further comprises recommending a treatment regimen for malignant mesothelioma for a subject for whom the level of bound antibody exceeds the predetermined level.

In some embodiments, an immunoassay kit is provided. The kit can comprise at least one of: (a) a first antibody that binds specifically to hyper-acetylated HMGB1; (b) a first antibody that binds specifically to disulfide HMGB1 (HMGB1C23-C45); (c) a first antibody that binds specifically to hypo-acetylated HMGB1; (d) a first antibody that binds specifically to fully reduced HMGB1; (e) a first antibody that binds specifically to hyper-acetylated disulfide HMGB1; (f) a first antibody that binds specifically to hyper-acetylated fully reduced HMGB1; (g) a first antibody that binds specifically to hypo-acetylated disulfide HMGB1; or (h) a first antibody that binds specifically to hypo-acetylated fully-reduced HMGB1. The kit can comprise a first detectable moiety. In some embodiments, the immunoassay comprises an ELISA kit. In some embodiments, the immunoassay comprises an ELISA substrate. In some embodiments, the immunoassay comprises a lateral flow assay kit. In some embodiments, the immunoassay comprises a lateral flow assay substrate. In some embodiments, the first antibody that binds specifically to hyper-acetylated HMGB1 binds specifically to an acetylated lysine in NLS1 or an acetylated lysine in NLS2 of HMGB1. In some embodiments, the first antibody that binds specifically to hyper-acetylated HMGB1 binds specifically to one of acetylated K30, acetylated K43, acetylated K90, or acetylated K141 of HMGB1. In some embodiments, the first antibody that binds specifically to disulfide HMGB1 binds specifically to a C23 and/C45 of HMGB1 when C23 and C45 of HMGB1 are joined by a disulfide bond. In some embodiments, the first antibody that binds specifically to fully reduced HMGB1 binds specifically to at least one of reduced C23, reduced C45, or reduced C106 of HMGB1. In some embodiments, the kit further comprises a second antibody that binds specifically to HMGB1. In some embodiments, the second antibody is immobilized on a substrate. In some embodiments, the first antibody is immobilized on a substrate. In some embodiments, the second antibody comprises the detectable moiety. In some embodiments, the kit further comprises a third antibody that binds specifically to the second antibody, in which the third antibody comprises the detectable moiety. In some embodiments, the first antibody comprises the detectable moiety. In some embodiments, the kit further comprises a third antibody that binds specifically to the first antibody, in which the third antibody comprises the detectable moiety. In some embodiments, the first antibody comprises a monoclonal antibody. In some embodiments, the first antibody comprises a polyclonal antibody. In some embodiments, the immunoassay comprises no wash assay. In some embodiments, the kit further comprises a second detectable moiety, wherein the first detectable moiety and the second detectable moiety comprise a FRET pair. In some embodiments, the first antibody comprises a non-native antibody. In some embodiments, the second antibody comprises a polyclonal antibody. In some embodiments, the third antibody comprises a polyclonal antibody. In some embodiments, the first antibody was engineered against an HMGB1 isoform.

In some embodiments, a kit for detecting at least one HMGB1 isoform by mass spectrometry is provided. The kit can comprise a first standard consisting essentially of a single isoform of HMGB1, wherein the single isoform is selected from: (a) a 24.585 KDa isoform of HMGB1; (b) a 24.587 KDa isoform of HMGB1; (c) a 25.467 KDa isoform of HMGB1; and (d) a 25.469 KDa isoform of HMGB1. In some embodiments, the kit further comprises a second standard consisting essentially of a single isoform of HMGB1, wherein the single isoform is selected from: (a) a 24.585 KDa isoform of HMGB1; (b) a 24.587 KDa isoform of HMGB1; (c) a 25.467 KDa isoform of HMGB1; and (d) a 25.469 KDa isoform of HMGB1, in which the second standard consists essentially of a different HMGB1 isoform than the first standard. In some embodiments, the kit further comprises an antibody that binds specifically to HMGB1. By way of example, a specific antibody against total HMGB1 can be used to pull down HMGB1 for further mass spectrometry analysis to identify HMGB1 isoforms in accordance with some embodiments herein.

### BRIEF DESCRIPTION OF THE DRAWINGS

FIGS. 1A-1L are a series of graphs showing, for example, that HMGB1 is highly expressed in malignant mesothelioma (MM) serum samples of MM patients, as can be ascertained in accordance with some embodiments herein.

FIG. 1A is a graph showing the total amount of HMGB1 in healthy subjects, MM patients and asbestos-exposed individuals, as can be ascertained in accordance with some embodiments herein.

FIG. 1B is a graph showing the types of HMGB1 isoforms detected in asbestos-exposed individuals by Electrospray ionization liquid chromatography mass spectrometry (ESI-LC-MS), as can be ascertained in accordance with some embodiments herein.

FIG. 1C is a graph showing the types of HMGB1 isoforms detected in MM individuals by ESI-LC-MS, as can be ascertained in accordance with some embodiments herein.

FIG. 1D is a graph showing the amounts of hyper-acetylated HMGB1 and hypo-acetylated HMGB1 isoforms in healthy subjects, MM patients, and asbestos-exposed individuals, as can be ascertained in accordance with some embodiments herein.

FIG. 1E is a graph showing the ratio of hyper-acetylated HMGB1 and hypo-acetylated HMGB1 in MM patients and asbestos-exposed individuals, in accordance with some embodiments herein.

FIG. 1F is a graph showing the amount of disulfide HMGB1 and fully reduced HMGB1 in healthy subjects, MM patients, and asbestos-exposed individuals, as can be ascertained in accordance with some embodiments herein.

FIG. 1G is a graph showing HMGB1 levels compared in healthy control cohort, asbestos-exposed individuals, and in MM patients, as can be ascertained in accordance with some embodiments herein.

FIG. 1H is a graph showing hypo-acetylated HMGB1 and hyper-acetylated HMGB1 levels compared in healthy control cohort, asbestos-exposed individuals, and in MM patients, as can be ascertained in accordance with some embodiments herein.

FIG. 1I is a graph showing fully-reduced HMGB1 and disulfide HMGB1 levels compared in healthy control cohort, asbestos-exposed individuals, and in MM patients, as can be ascertained in accordance with some embodiments herein.

FIG. 1J is a graph of mass spectra, showing hypo-acetylated HMGB1 levels upon exposure to asbestos fibers, as can be ascertained in accordance with some embodiments herein. A mass of 24,585 KDa corresponds to disulfide hypo-acetylated HMGB1, and a mass of 24,587 KDa corresponds to fully reduced hypo-acetylated HMGB1.

FIG. 1K is a graph of mass spectra, showing hyper-acetylated HMGB1 levels in the supernatant from Ren MM cell cultures in accordance with methods and kits of some embodiments herein. A mass of 24,585 KDa corresponds to disulfide hypo-acetylated HMGB1; a mass of 24,587 KDa corresponds to fully reduced hypo-acetylated HMGB1; a mass of 24,731 KDa corresponds to sulphonyl hypo-acetylated HMGB1; a mass of 25,467 KDa corresponds to disulfide acetylated HMGB1; a mass of 25,469 KDa corresponds to fully reduced acetylated HMGB1; and a mass of 25,613 KDa corresponds to sulphonyl acetylated HMGB1.

FIG. 1L is a graph of mass spectra, showing hyper-acetylated HMGB1 levels in the supernatant from Hmeso MM cell cultures, as can be ascertained in accordance with some embodiments herein. A mass of 24,585 KDa corresponds to disulfide hypo-acetylated HMGB1; a mass of 24,587 KDa corresponds to fully reduced hypo-acetylated HMGB1; a mass of 24,731 KDa corresponds to sulphonyl hypo-acetylated HMGB1; a mass of 25,467 KDa corresponds to disulfide acetylated HMGB1; a mass of 25,469 KDa corresponds to fully reduced acetylated HMGB1; and a mass of 25,613 KDa corresponds to sulphonyl acetylated HMGB1.

FIGS. 2A-2I are a series of graphs showing, for example, the performance of total and isoform-specific HMGB1 as biomarkers to discriminate MM patients from asbestos-exposed individuals, for example in accordance with some embodiments herein.

FIG. 2A is a graph showing the area under the ROC curve (AUC), sensitivity at 100% specificity, specificity at 100% sensitivity of total HMGB1 in accordance with some embodiments herein.

FIG. 2B is a graph showing AUC, sensitivity at 100% specificity, specificity at 100% sensitivity of hyper-acetylated HMGB1 in accordance with some embodiments herein.

FIG. 2C is a graph showing AUC, sensitivity at 100% specificity, specificity at 100% sensitivity for disulfide HMGB1 in accordance with some embodiments herein.

FIG. 2D is a graph showing AUC, sensitivity at 100% specificity, specificity at 100% sensitivity for fibulin-3.

FIG. 2E is a graph showing AUC, sensitivity at 100% specificity, specificity at 100% sensitivity for osteopontin.

FIG. 2F is a graph showing AUC, sensitivity at 100% specificity, specificity at 100% sensitivity for SMRP.

FIG. 2G is a graph showing the levels of isoform-specific HMGB1, as can be ascertained in accordance with some embodiments herein, compared to levels of fibulin-3.

FIG. 2H is a graph showing the levels of isoform-specific HMGB1, as can be ascertained in accordance with some embodiments herein, compared to levels of osteopontin.

FIG. 2I is a graph showing the levels of isoform-specific HMGB1, as can be ascertained in accordance with some embodiments herein, compared to levels of SMRP.

FIGS. 3A-3C are a series of graphs and pictures showing, for example, detection of high levels of hyper-acetylated and hypo-acetylated HMGB1 in MM cells, as can be ascertained in accordance with some embodiments herein.

FIG. 3A is a graph of mass spectra, showing detection of hypo-acetylated HMGB1 in crocidolite asbestos-exposed HM (Asb-HM) cultures, as can be ascertained in accordance with some embodiments herein.

FIG. 3B is a graph of mass spectra, showing detection of both hyper-acetylated and hypo-acetylated HMGB1 in MM cells, as can be ascertained in accordance with some embodiments herein.

FIG. 3C is a graph showing levels of HMGB1 isoforms in supernatant of primary HM culture, Asb-HM, and MM cells, as can be ascertained in accordance with some embodiments herein.

FIGS. 3D-3F is a series of microscope pictures (40×) of HM, Asb-HM, and MM cells showing respectively classical cobblestone morphology in normal culture (HM) (FIG. 3D), or dying HM due to asbestos exposure (Asb-HM) (FIG. 3E), and MM cells with spindle-like morphology (MM) (FIG. 3F), as can be ascertained in accordance with some embodiments herein.

FIGS. 4A-4F are a series of graphs showing, for example, that HMGB1 is highly expressed in malignant mesothelioma (MM) serum samples of MM patients in accordance with methods and kits of some embodiments herein.

FIG. 4A is a graph showing HMGB1 levels compared in healthy control cohort, asbestos-exposed individuals, and in MM patients in accordance with methods and kits of some embodiments herein.

FIG. 4B is a graph showing hyper-acetylated HMGB1 levels compared in healthy control cohort, asbestos-exposed individuals, and in MM patients in accordance with methods and kits of some embodiments herein.

FIG. 4C is a graph showing AUC, sensitivity at 100% specificity, specificity at 100% sensitivity of total HMGB1 in accordance with methods and kits of some embodiments herein.

FIG. 4D is a graph showing AUC, sensitivity at 100% specificity, specificity at 100% sensitivity of hyper-acetylated HMGB1 in accordance with methods and kits of some embodiments herein.

FIG. 4E is a graph showing total HMGB1 levels compared in patients with early (Stages I-II) and late stage of MM (Stage III-IV) in accordance with methods and kits of some embodiments herein.

FIG. 4F is a graph showing hyper-acetylated HMGB1 levels compared in compared in patients with early (Stages I-II) and late stage of MM (Stage III-IV) in accordance with methods and kits of some embodiments herein.

FIGS. 5A-5D are a series of graphs showing, for example, that MM patients had significantly higher levels of total HMGB1 compared to patients with cytologically benign pleural effusions and malignant (non-MM) pleural effusions in accordance with methods and kits of some embodiments herein.

FIG. 5A is a graph showing total HMGB1 levels compared in MM patients, patients with cytologically benign pleural effusions, and patients with malignant (non-MM) pleural effusions in accordance with methods and kits of some embodiments herein.

FIG. 5B is a graph showing hyper-acetylated HMGB1 levels compared in MM patients, patients with cytologically benign pleural effusions, and patients with malignant (non-MM) pleural effusions in accordance with methods and kits of some embodiments herein.

FIG. 5C is a graph showing AUC, sensitivity at 100% specificity, specificity at 100% sensitivity of total HMGB1 in accordance with methods and kits of some embodiments herein.

FIG. 5D is a graph showing AUC, sensitivity at 100% specificity, specificity at 100% sensitivity of hyper-acetylated HMGB1 in accordance with methods and kits of some embodiments herein.

FIGS. 6A-6L are a series of graphs showing, for example, levels of Mesothelin, and OPN in MM patients, patients with cytologically benign pleural effusions, and patients with malignant (non-MM) pleural effusions in accordance with methods and kits of some embodiments herein.

FIG. 6A is a graph showing levels of fibulin-3 in MM patients and asbestos-exposed individuals in accordance with methods and kits of some embodiments herein.

FIG. 6B is a graph showing levels of mesothelin in MM patients and asbestos-exposed individuals in accordance with methods and kits of some embodiments herein.

FIG. 6C is a graph showing levels of OPN in MM patients and asbestos-exposed individuals in accordance with methods and kits of some embodiments herein.

FIG. 6D is a graph showing levels of Fibulin-3 in MM patients and asbestos-exposed individuals in accordance with methods and kits of some embodiments herein.

FIG. 6E is a graph showing AUC, sensitivity at 100% specificity, specificity at 100% sensitivity of mesothelin in accordance with methods and kits of some embodiments herein.

FIG. 6F is a graph showing AUC, sensitivity at 100% specificity, specificity at 100% sensitivity of OPN in accordance with methods and kits of some embodiments herein.

FIG. 6G is a graph showing levels of fibulin-3 in MM patients (“MM”), patients with cytologically benign pleural effusions (“Ben-PE”), and malignant (non-MM) pleural effusions (“Mal-PE”).

FIG. 6H is a graph showing levels of mesothelin in MM patients (“MM”), patients with cytologically benign pleural effusions (“Ben-PE”) and malignant (non-MM) pleural effusions (“Mal-PE”).

FIG. 6I is a graph showing levels of OPN in MM patients (“MM”), patients with cytologically benign pleural effusions (“Ben-PE”) and malignant (non-MM) pleural effusions (“Mal-PE”).

FIG. 6J is a graph showing AUC, sensitivity at 100% specificity, specificity at 100% sensitivity of fibulin-3.

FIG. 6K is a graph showing AUC, sensitivity at 100% specificity, specificity at 100% sensitivity of mesothelin.

FIG. 6L is a graph showing AUC, sensitivity at 100% specificity, specificity at 100% sensitivity of osteopontin.

FIGS. 7A-7B are graphs showing, for example, correlations between different ways of measuring the levels of mesothelin and HMGB1 in accordance with methods and kits of some embodiments herein.

FIG. 7A is a graph showing correlation of mesothelin levels measured using an FDA-approved MESOMARK™ kit (SMRP Fujirebio) and an R&D Systems mesothelin ELISA kit (Mesothelin R&D).

FIG. 7B is a graph showing correlation of total HMGB1 levels measured using a commercially available ELISA kit and MS protocol in accordance with methods and kits of some embodiments herein.

FIG. 8 is a graph showing relative percentages of hyper-acetylated and hypo-acetylated HMGB1 in sera of asbestos-exposed individuals and MM patients in accordance with methods and kits of some embodiments herein.

FIGS. 9A-9D are a series of graphs showing AUC curves, for example, that total HMGB1 is a reliable biomarker to discriminate individuals with asbestos-exposure and/or MM from healthy controls.

FIG. 9A is a graph showing graph showing AUC, sensitivity at 100% specificity, specificity at 100% sensitivity of total HMGB1 in asbestos-exposed individuals as compared to healthy controls in accordance with methods and kits of some embodiments herein.

FIG. 9B is a graph showing AUC, sensitivity at 100% specificity, specificity at 100% sensitivity of hyper-acetylated HMGB1 in asbestos-exposed individuals as compared to healthy controls in accordance with methods and kits of some embodiments herein.

FIG. 9C is a graph showing AUC, sensitivity at 100% specificity, specificity at 100% sensitivity of total HMGB1 in MM patients as compared to healthy controls in accordance with methods and kits of some embodiments herein.

FIG. 9D is a graph showing AUC, sensitivity at 100% specificity, specificity at 100% sensitivity of hyper-acetylated HMGB1 in MM patients as compared to healthy controls in accordance with methods and kits of some embodiments herein.

### DETAILED DESCRIPTION

Different isoforms of HMGB1, including hyper-acetylated HMGB1, disulfide HMGB1 (HMGB1C23-C45), hypo-acetylated HMGB1, fully reduced HMGB1, hyper-acetylated disulfide HMGB1, hyper-acetylated fully reduced HMGB1, hypo-acetylated disulfide HMGB1, and hypo-acetylated fully-reduced HMGB1, or signatures based on the presence or levels of some of these isoforms can identify a subject as having asbestos exposure, malignant mesothelioma or an inflammatory cancer. Furthermore, total HMGB1 can be used to differentiate asbestos-exposed individuals from non-exposed people. Moreover, specific HMGB1 isoforms can be used to differentiate malignant mesothelioma (MM) patients from asbestos-exposed individuals. Provided in accordance with some embodiments herein are antibodies that can bind to and detect particular isoforms of HMGB1. Also provided in accordance with some embodiments herein are mass spectrometry methods for identifying HMGB1 isoforms. Such antibodies, kits, and mass spectrometry methods can be useful for identifying HMGB1 isoform signatures characteristic of asbestos exposure, malignant mesothelioma or an inflammatory cancer in a subject. It is contemplated that detection of a level of antibody bound to a HMGB1 isoform or isoform in a sample can also be used to determine a level of HMGB1 isoform(s) in a sample. As such, it will be understood that when detection of a level of antibody bound to an HMGB1 isoform or isoforms is described herein, the skilled artisan can also determine a level of the HMGB1 isoform or isoforms in the sample, for example by comparing the levels to a suitable control or standard, such as a reference sample having a known level of the HMGB1 isoform(s), or a set of electronically stored or written values.

Early MM detection is associated with better responses to therapy and prolonged survival. The long latency period of 20-60 years between exposure to carcinogenic fibers and MM development provides physicians with a potential window for early detection and intervention. It is contemplated that detection of HMGB1 isoforms in accordance with some embodiments herein, and detection of HMGB1 isoform signatures in accordance with some embodiments herein can be useful in early detection of malignant mesothelioma, as well as inflammatory cancers such as colorectal cancer, pancreatic cancer, breast cancer, liver cancer, and the like.

Without being limited by any theory, it is contemplated herein that HMGB1 can be involved in asbestos-induced MM initiation and progression, and that serum HMGB1 levels are increased both in asbestos-exposed individuals and in patients with MM compared to healthy controls (see US Pub. No. 2014/0170685, hereby incorporated by reference in its entirety).

As used herein, “subject” includes organisms which are capable of suffering from asbestos exposure, mesothelioma, or an inflammatory cancer, such as human and non-human animals. Optionally, the subject comprises a human. The term “non-human animals” includes all vertebrates, for example, mammals, e.g., rodents (e.g., mice, rats, Guinea Pigs, etc.), rabbits, canines, ruminants (e.g., sheep, cows, etc.), non-human primates, and non-mammals, such as chickens, amphibians, reptiles, etc. In the case of the non-human animals, the subject can be a laboratory animal, including an engineered animal, for example a mouse engineered to have a cancer tissue.

### HMGB1 and HMGB1 Isoforms

HMGB1 is a damage-associated molecular pattern (DAMP) molecule and a mediator of chronic inflammation (Bianchi M E (2007) DAMPs, PAMPs and alarmins: all we need to know about danger. J Leukoc Biol 81: 1-5; 13-15; which is incorporated herein by reference in its entirety). HMGB1 is actively secreted by macrophages and dendritic cells (DCs) and passively released by cells undergoing necrosis. HMGB1 is a nuclear protein, but can be detected in the cytoplasm of cells undergoing necrosis and in cells that actively secrete HMGB1, such as macrophages. HMGB1 binds to the Receptor for Advanced Glycation Endproducts (RAGE) and to the Toll-like Receptors (TLRs) 2 and 4, responsible for inflammatory responses. The activation of RAGE by HMGB1 induces tumor cell proliferation, migration, and invasion. HMGB1 induces migration in certain cell types. Wild-type human HMGB1 has the amino acid sequence:

(SEQ ID NO: 1)
MGKGDPKKPRGKMSSYAFFVQTCREEHKKKHPDASVNFSEFSKKCSERWK
TMSAKEKGKFEDMAKADKARYEREMKTYIPPKGETKKKFKDPNAPKRPPS
AFFLFCSEYRPKIKGEHPGLSIGDVAKKLGEMWNNTAADDKQPYEKKAAK
LKEKYEKDIAAYRAKGKPDAAKKGVVKAEKSKKKKEEEEDEEDEEDEEEE
EDEEDEDEEEDDDDE.

Without being limited by any theory, it is contemplated that when asbestos is deposited in the pleura, mesothelial cells and reactive macrophages attempt to phagocyte these fibers and as they cannot “digest” these fibers they undergo programmed cell necrosis and release HMGB1. HMGB1 attracts more mesothelial cells and macrophages and propagates this process that continues over time as asbestos fibers lodge in the tissues. Mesothelioma cells that grow out of a HMGB1 rich environment are often “addicted” to HMGB1, and accordingly actively secrete and require HMGB1 to sustain their own growth (Carbone and Yang, “Molecular pathways: targeting mechanisms of asbestos and erionite carcinogenesis in mesothelioma” Clinical Cancer Res 2012, 18: 598-604.).

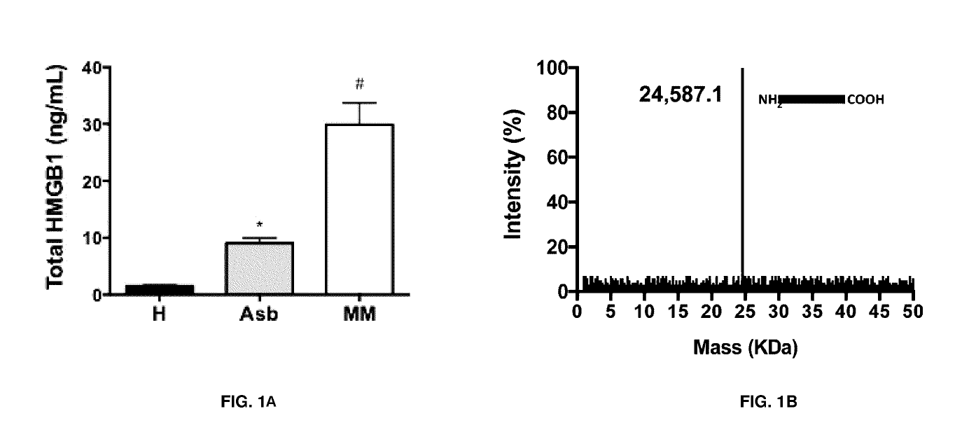

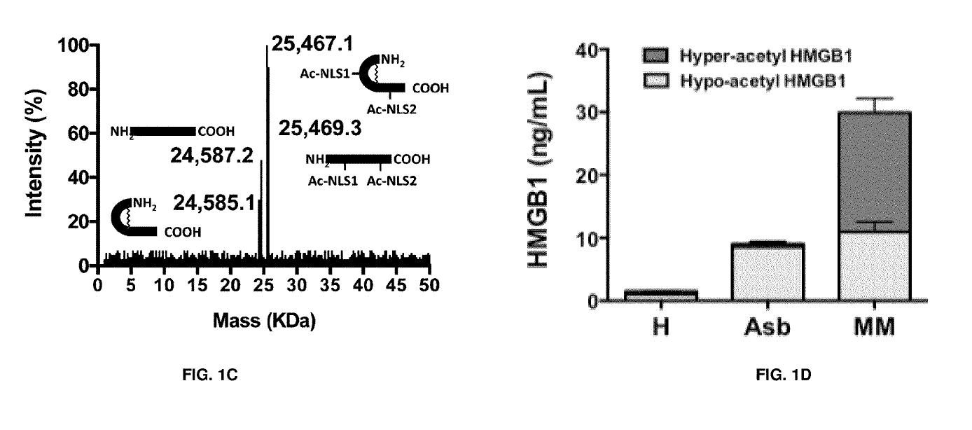

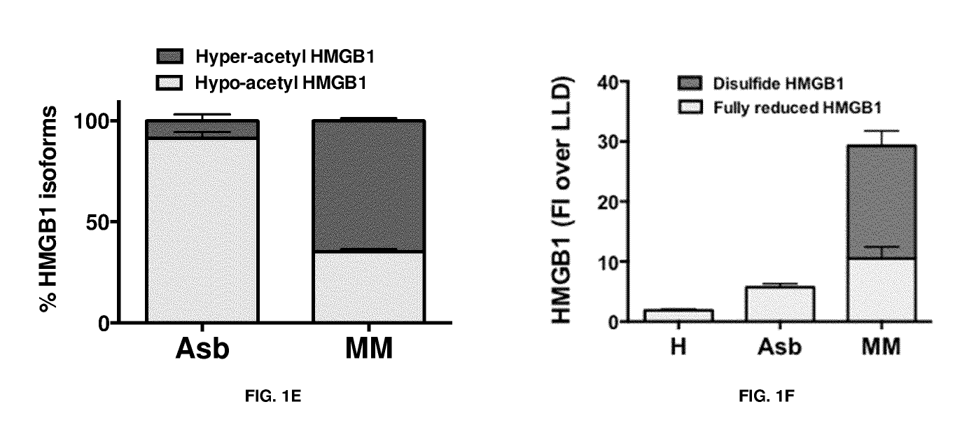

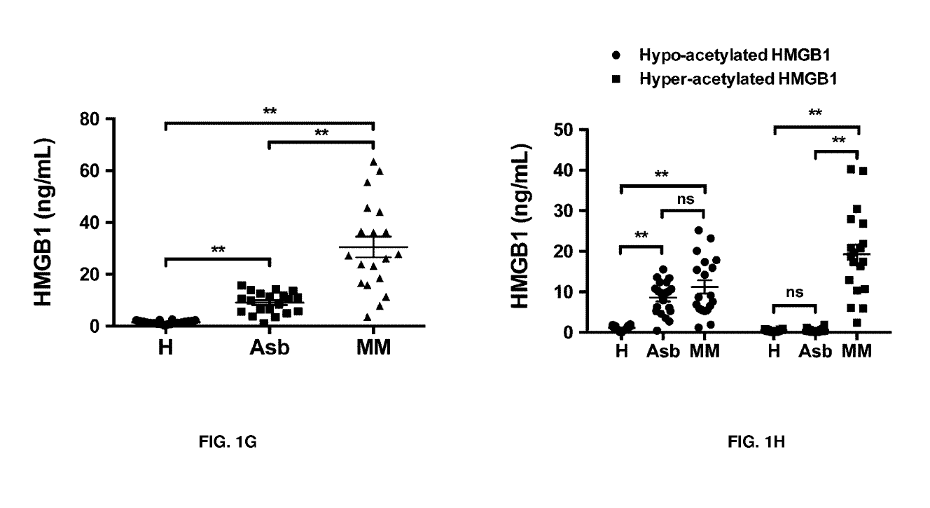

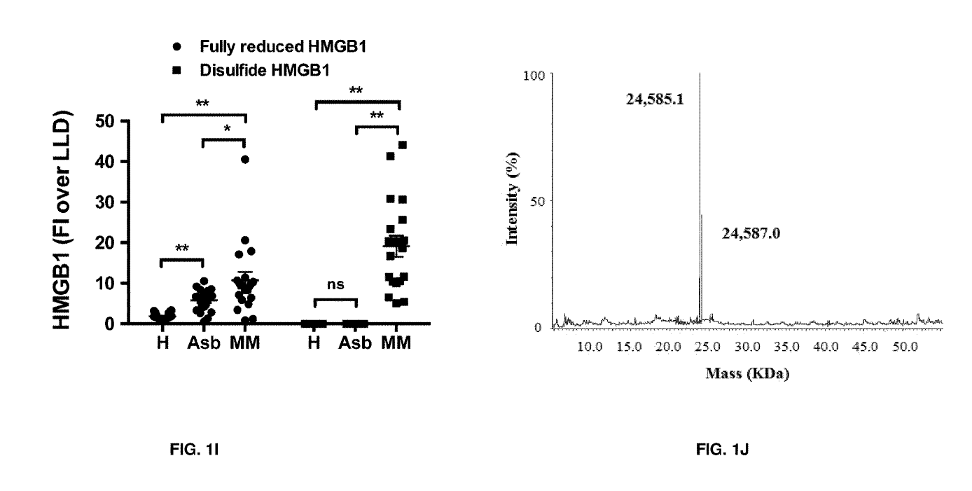

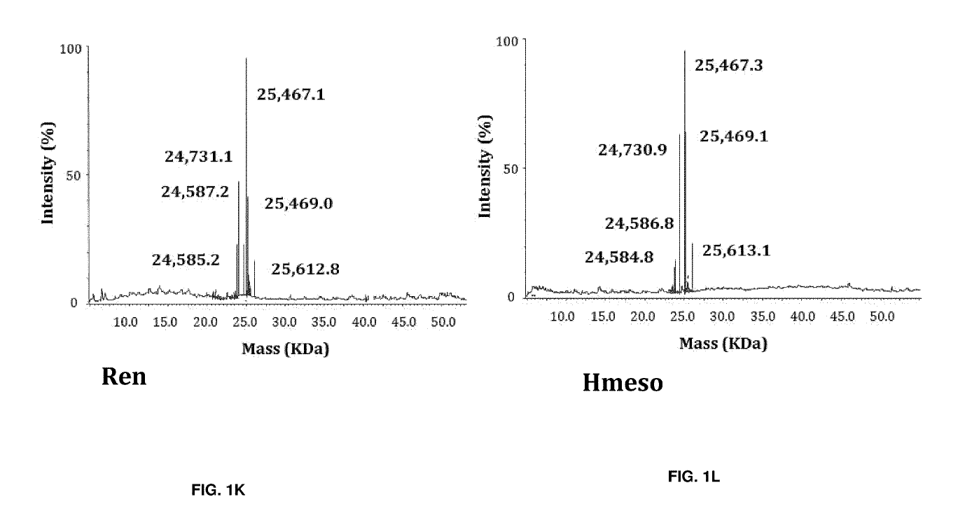

A number of different HMGB1 isoforms have been observed, and can be detected in accordance with some embodiments herein. By way of example, HMGB1 can be hyper-acetylated at one or more lysine residues in its two nuclear localization signals NLS1 and NLS2, for example K30, K43, K90, and or K141. As used herein, “hyper-acetylated” HMGB1 refers to isoforms of HMGB1 with acetylation at two or more positions. HMGB1 can also undergo redox-sensitive modifications of its three cysteines. As used herein, “hypo-acetylated” HMGB1 is synonymous with “non-acetylated” HMGB1, these two terms may be used interchangeably. As such, for “hypo-acetylated” HMGB1 (a.k.a. “non-acetylated HMGB1), there is no acetylation of any of the lysine residues in NLS1, and no acetylation of any of the lysine residues of NLS2. An unreduced form of HMGB1 comprises a disulfide bond between C23 and C45 (this form is also referred to herein as “HMGB1 C23-C45”). On the other hand, this disulfide bond is absent in fully reduced HMGB1 isoforms. Disulfide HMGB1 have been observed to have cytokine activity, while fully reduced HMGB1 has been observed to have chemotactic activity. Accordingly, “fully reduced” HMGB1 has been referred to as “chemokine HMGB1”. An additional HMGB1 isoform, HMGB1 with all cysteine residues terminally oxidized to sulphonates (“sulphonyl HMGB1”) appears to be immunologically inert. Different HMGB1 isoforms in accordance with embodiments herein have also been observed to have characteristic molecular masses. For example, disulfide hypo-acetylated HMGB1 has been observed to have a mass of 24,585.1 Da; fully reduced hypo-acetylated HMGB1 has been observed to have a mass of 24,587.2 Da; disulfide hyper-acetylated HMGB1 has been observed to have a mass of 25,467.1 Da; and fully reduced hyper-acetylated HMGB1 has been observed to have a mass of 25,469.3 (see, e.g FIG. 1C).

It has been observed that different HMGB1 isoforms are present in different stages of asbestos-induced MM pathogenesis. Additionally, primary human mesothelial cells (HM) release negligible amounts of HMGB1, and the total and isoform-specific HMGB1 can be analyzed in concentrated supernatants of cell cultures mimicking different stages of MM pathogenesis (see FIGS. 1A-1L) Without being limited by any theory, it has been observed that hyper-acetylated HMGB1 is actively secreted, while hypo-acetylated HMGB1 is passively released upon necrotic cell death. Fully reduced HMGB1 can have chemotactic activity, while disulfide HMGB1 (HMGB1C23-C45) can have cytokine activity.

It has been observed herein that hyper-acetylated isoforms of HMGB1 have a greater molecular mass than corresponding hypo-acetylated isoforms (see FIG. 1C). It has been observed herein that fully reduced isoforms of HMGB1 have a greater molecular mass than corresponding disulfide isoforms (see FIG. 1C). As such, it is contemplated that in order of lowest molecular mass to greatest molecular mass, several isoforms of HMGB1 (assuming no other differences) are as follows: (lowest molecular mass) disulfide hypo-acetylated<fully reduced hypo-acetylated<disulfide hyper-acetylated<fully reduced hyper-acetylated (greatest molecular mass). Accordingly, it is contemplated that in accordance with some embodiments herein, isoforms of HMGB1 can be differentiated based on molecular mass. By way of example, molecular masses that can differentiate HMGB1 isoforms in accordance with some embodiments herein are disulfide hypo-acetylated (24,585.1 Da)<fully reduced hypo-acetylated (24,587.2 Da)<disulfide hyper-acetylated (25,467.1 Da)<fully reduced hyper-acetylated (25,469.3) (see, e.g. FIG. 1C, summarizing molecular masses of various HMGB1 isoforms were determined by Electrospray ionization liquid chromatography mass spectrometry (ESI-LC-MS)). In some embodiments, HMGB1 isoforms are differentiated by mass spectrometry. In some embodiments, HMGB1 isoforms are differentiated by antibodies that bind to epitopes characteristic of each isoform.

### Antibodies

As used herein, “antibody” refers to a capture agent that comprises at least an epitope binding domain of an antibody. These terms are well understood by those skilled in the art, and refer to a protein comprising one or more polypeptides that specifically binds an antigen. The basic structural unit of a full-length antibody is known, and includes a tetramer and consists of two identical pairs of antibody chains, each pair having one light and one heavy chain. In each pair, the light and heavy chain variable regions are together responsible for binding to an antigen, and the constant regions are responsible for the antibody effector functions. Antibodies useful in accordance with some embodiments herein also encompass immunoglobulin molecules and immunologically active fragments of immunoglobulin molecules, i.e., molecules that contain an antigen binding site, these fragments may or may not be fused to another immunoglobulin domain including but not limited to, an Fc region or fragment thereof. As outlined herein, the terms “antibody” and “antibodies” include full length antibodies and Fc variants thereof comprising Fc regions, or fragments thereof, comprising at least one novel amino acid residue described herein fused to an immunologically active fragment of an immunoglobulin or to other proteins as described herein. Such variant Fc fusions include but are not limited to, scFv-Fc fusions, variable region (e.g., VL and VH)-Fc fusions, scFv-scFv-Fc fusions. Immunoglobulin molecules can be of any type (e.g., IgG, IgE, IgM, IgD, IgA and IgY), class (e.g., IgG1, IgG2, IgG3, IgG4, IgA1 and IgA2) or subclass.

As used herein “specifically”, “specifically bind”, “preferentially”, and “preferentially bind”, including variations of these root terms denote that the antibody has a dissociation constant (K_{D}=k_{off}/kₒₙ) of less than or equal to 10⁻⁵ M. As such, in some embodiments, the antibody has a dissociation constant (K_{D}) for the indicated epitope of less than or equal to 10⁻⁵ M, for example less than or equal to 10⁻⁶ M, less than or equal to 10⁻⁷ M, less than or equal to 10⁻⁸ M, less than or equal to 10⁻⁹ M, or less than or equal to less than or equal to 10⁻¹⁰ M, including ranges better any two of the listed values. As used herein, an antibody that binds specifically to a particular isoform of HMGB1, or an epitope characteristic of a particular isoform of HMGB1 is understood to not bind to different isoforms of HMGB1 (or epitopes characteristic of those other isoforms). For example, an antibody that binds specifically to hyper-acetylated HMGB1 is understood not to bind specifically to hypo-acetylated HMGB1. For example, an antibody that binds specifically to hypo-acetylated HMGB1 is understood not to bind specifically to hyper-acetylated HMGB1. For example, an antibody that binds specifically to disulfide HMGB1 is understood not to bind specifically to fully reduced HMGB1. For example, an antibody that binds specifically to fully reduced HMGB1 is understood not to bind specifically to disulfide HMGB1.

Antibodies useful in accordance with methods and kits in accordance with some embodiments herein include, but are not limited to, synthetic antibodies, monoclonal antibodies, recombinantly produced antibodies, intrabodies, multispecific antibodies (including bi-specific antibodies), human antibodies, humanized antibodies, chimeric antibodies, synthetic antibodies, single-chain Fvs (scFv), Fab fragments, Fab fragments, disulfide-linked Fvs (sdFv) (including bi-specific sdFvs), and anti-idiotypic (anti-Id) antibodies, and epitope-binding fragments of any of the above. In some embodiments, the antibody is a monoclonal antibody. In some embodiments, the antibody is a polyclonal antibody.

Antibodies useful in accordance with some embodiments herein include monospecific antibodies, bispecific antibodies, trispecific antibodies or antibodies of greater multispecificity. Multispecific antibodies can be specific for different epitopes of a polypeptide or may be specific for both a polypeptide as well as for a heterologous epitope, such as a heterologous polypeptide or solid support material. See, e.g., PCT Publication Nos. WO 93/17715; WO 92/08802; WO91/00360; WO 92/05793; Tutt, et al., J. Immunol. 147:60-69 (1991); U.S. Pat. Nos. 4,474,893; 4,714,681; 4,925,648; 5,573,920; 5,601,819; and Kostelny et al., J. Immunol. 148:1547-1553 (1992); each of which is incorporated herein by reference in its entirety.

In some embodiments, the antibodies have half-lives (e.g., serum half-lives) in a mammal, (e.g., a human), of greater than 5 days, greater than 10 days, greater than 15 days, greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies in a mammal, (e.g., a human), results in a higher serum titer of said antibodies or antibody fragments in the mammal, and thus, reduces the frequency of the administration of said antibodies or antibody fragments and/or reduces the concentration of said antibodies or antibody fragments to be administered. Antibodies having increased in vivo half-lives can be generated by techniques known to those of skill in the art. For example, antibodies with increased in vivo half-lives can be generated by modifying (e.g., substituting, deleting or adding) amino acid residues identified as involved in the interaction between the Fc domain and the FcRn receptor (see, e.g., International Publication Nos. WO 97/34631; WO 04/029207; U.S. Pat. No. 6,737,056 and U.S. Patent Publication No. 2003/0190311); each of which is incorporated herein by reference in its entirety.

In some embodiments, an antibody is a non-human antibody, for example mouse, rat, guinea pig, rabbit, donkey, goat, horse, pig, and the like. For some uses, for example in vivo use of antibodies in humans and in vitro methods and kits in accordance with some embodiments herein, it may be desirable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See, e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397; each of which is incorporated herein by reference in its entirety. Humanized antibodies are antibody molecules from non-human species antibody that bind the desired antigen having one or more complementarity determining regions (CDRs) from the non-human species and a framework region from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988); each of which is incorporated herein by reference in its entirety). Antibodies can be humanized using a variety of techniques known in the art including, for example, CDR-grafting (EP 239,400; PCT publication WO 91/09967; U.S. Pat. Nos. 5,225,539; 5,530,101; and 5,585,089; each of which is incorporated herein by reference in its entirety), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332); each of which is incorporated herein by reference in its entirety.

In some embodiments, the antibody comprises a non-native antibody (i.e. an antibody from a host other than the subject from which a sample came). In some embodiments, the antibody is from a host of the same species as the subject. In some embodiments, the antibody is from a host of a different species than the subject.

Antibodies in accordance with kits and methods in accordance with some embodiments herein can bind specifically to an HMGB1 isoform of interest. Examples of isoforms that the antibody can specifically bind include hyper-acetylated HMGB1, hypo-acetylated HMGB1, disulfide HMGB1, fully-reduced HMGB1, hyper-acetylated disulfide HMGB1, hypo-acetylated disulfide HMGB1, hyper-acetylated fully-reduced HMGB1, or hypo-acetylated fully-reduced HMGB1. The antibody can bind specifically to one of the indicated isoforms, while not appreciably binding to the other isoforms. Additionally, antibodies that bind to HMGB1 in general, but do not necessarily bind to specific isoforms of HMGB1 can be useful in accordance with some embodiments, for example as capture antibodies (or detection antibodies if a capture antibody binds specifically to a particular isoform), controls, and the like.

Optionally, the antibody comprises a monoclonal antibody that recognizes an epitope characteristic of the HMGB1 isoform. Optionally, the antibody can bind specifically to the epitope characteristic of the HMGB1 isoform, while not appreciably binding to epitopes characteristic of other HMGB1 isoforms. Examples of epitopes characteristic of hyper-acetylated isoforms of HMGB1 that can be bound by the antibody include epitopes comprising acetylated K30, acetylated K43, acetylated K90, acetylated K141, or two or more of these, for example acetylated K30 and acetylated K43, acetylated K30 and acetylated K90, acetylated K30 and acetylated K141, acetylated K43 and acetylated K90, acetylated K43 and acetylated K141, acetylated K90 and acetylated K141, acetylated K30 and acetylated K43 and acetylated K90, acetylated K30 and acetylated K90 and acetylated K141, acetylated K30 and acetylated K43 and acetylated K141, acetylated K43 and acetylated K90 and acetylated K14, or acetylated K30 and acetylated K43 and acetylated K90 and acetylated K141. Examples of epitopes characteristic of the hypo-acetylated isoforms of HMGB1 can be bound by the antibody in accordance with embodiments herein include epitopes comprising include epitopes comprising hypo-acetylated K30 and hypo-acetylated K43, hypo-acetylated K30 and hypo-acetylated K90, hypo-acetylated K30 and hypo-acetylated K141, hypo-acetylated K43 and hypo-acetylated K90, hypo-acetylated K43 and hypo-acetylated K141, hypo-acetylated K90 and hypo-acetylated K141, hypo-acetylated K30 and hypo-acetylated K43 and hypo-acetylated K90, hypo-acetylated K30 and hypo-acetylated K90 and hypo-acetylated K141, hypo-acetylated K30 and hypo-acetylated K43 and hypo-acetylated K141, hypo-acetylated K43 and hypo-acetylated K90 and hypo-acetylated K14, or hypo-acetylated K30 and hypo-acetylated K43 and hypo-acetylated K90 and hypo-acetylated K141. Examples of epitopes characteristic of the disulfide isoforms of HMGB1 that can be bound by the antibody include epitopes comprising a disulfide bond between C23 and C45. Examples of epitopes characteristic of the fully reduced isoforms of HMGB1 that can be bound by the antibody include reduced C23, reduced C45, or reduced C106, or combinations of these, for example reduced C23 and reduced C45, reduced C23 and reduced C106, reduced C45 and reduced C106, or reduced C23 and reduced C45 and reduced C106.

In some embodiments, an antibody that binds specifically to a particular isoform of HMGB1 binds to an epitope characteristic of that isoform, for example a hyper-acetylated lysine, a hypo-acetylated lysine, a reduced cysteine, or a disulfide bond as described herein. The antibody can bind specifically to the indicated particular HMGB1 isoform, while not binding appreciably to other isoforms of HMGB1. Optionally, the antibody is a monoclonal antibody. Methods for making monoclonal antibodies are well known in the art. For example, monoclonal antibodies can be created through the recovery (cloning or identification) of the binding domain of an individual immunoglobulin from a larger polyclonal response. Antibodies can be raised in a variety of hosts, for example mouse, rat, guinea pig, rabbit, donkey, goat, horse, pig, and the like, by administering one or more regimens of antigen (for example, a particular HMGB1 isoform) to the host. For example, the predominant heavy chain variable region genes and predominant light chain variable region genes can be recovered from purified antibody-producing cells. These binding domains can be cloned by either immortalizing the cell (e.g., hybridoma fusion to a myeloma, or EBV immortalization), selection for binding from an in vitro display library (f-phage, phagemid, yeast, ribosome display, whole bacterial display, mammalian cell display), or direct cloning from individually sorted cells via RT-PCR amplification (limiting dilution, FACS sorting to wells).

Hybridoma techniques are well known in the art. A host animal is typically injected with the antigen, and, after a period of time, antibody-making cell can be isolated, usually from the spleen. The antibody-making cell can be fused with myeloma (or other immortalized cell) cells to provide fused cells, referred to as hybridomas. The hybridomas can be separated from unfused antibody-making cells and myeloma cells. Specific hybridomas can be isolated and tested to confirm that the isolated hybridoma produces antibody specific for the antigen used in the immunization step. The hybridoma so produced combines the ability of the parent antibody-making cell to produce a specific single antibody with the ability of its parent myeloma (or other immortalized) cell to continually grow and divide, either in vitro as a cell culture or in vivo as a tumor after injection into the peritoneal cavity of an animal. Hybridoma lines can be used, for example to produce monoclonal antibodies.

In some embodiments, monoclonal antibodies are created through the recovery (cloning or identification) of the binding domain of an individual immunoglobulin from a larger polyclonal response. For example, recovering the predominant heavy chain variable region genes and predominant light chain variable region genes from purified bulk antibody-producing cells. These binding domains can be cloned by either immortalizing the cell (e.g., Hybridoma fusion to a myeloma, or EBV immortalization), selection for binding from an in vitro display library (f-phage, phagemid, yeast, ribosome display, whole bacterial display, mammalian cell display), or direct cloning from individually sorted cells via RT-PCR amplification (limiting dilution, FACS sorting to wells).

### Detectable Moieties

As used herein, “detectable moiety” refers to a molecule or complex, the presence or absence of which can be determined by the presence or absence of a signal characteristic of that molecule or complex. A variety of detectable moieties can be suitable in accordance with embodiments herein, for example fluorophores, nanoparticles, radiolabels, enzyme-substrate pairs, members of FRET pairs, and the like.

In some embodiments, the detectable moiety comprises a fluorophore. Exemplary suitable fluorophores include, but are not limited to: xanthene dyes, e.g., fluorescein and rhodamine dyes, such as fluorescein isothiocyanate (FITC), 2-[ethylamino)-3-(ethylimino)-2-7-dimethyl-3H-xanthen-9-yl]benzoic acid ethyl ester monohydrochloride (R6G)(emits a response radiation in the wavelength that ranges from about 500 to 560 nm), 1,1,3,3,3′,3′-Hexamethylindodicarbocyanine iodide (HIDC) (emits a response radiation in the wavelength that ranged from about 600 to 660 nm), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3 (emits a response radiation in the wavelength that ranges from about 540 to 580 nm), Cy5 (emits a response radiation in the wavelength that ranges from about 640 to 680 nm), etc; BODIPY dyes and quinoline dyes. Specific fluorophores of interest include: Pyrene, Coumarin, Diethylaminocoumarin, FAM, Fluorescein Chlorotriazinyl, Fluorescein, R110, Eosin, JOE, R6G, HIDC, Tetramethylrhodamine, TAMRA, Lissamine, ROX, Napthofluorescein, Texas Red, Napthofluorescein, Cy3, and Cy5, and the like.

In some embodiments, the detectable moiety comprises a quencher. A quencher can absorb electromagnetic radiation and dissipate it as heat, thus remaining dark. Example quenchers that be used in accordance with some embodiments herein include Dabcyl, NFQ's, such as BHQ-1 or BHQ-2 (Biosearch), IOWA BLACK FQ (IDT), and IOWA BLACK RQ (IDT). In some embodiments, the quencher is selected to pair with a fluorophore so as to absorb electromagnetic radiation emitted by the fluorophore. Fluorophore/quencher pairs useful in the compositions and methods disclosed herein are well-known in the art, and can be found, e.g., described in S. Marras, “Selection of Fluorophore and Quencher Pairs for Fluorescent Nucleic Acid Hybridization Probes” available at the world wide web site molecular-beacons.org/download/marras,mmb06(335)3.pdf. As such, in some embodiments, the detectable moiety comprises a quencher (for example, the presence of the detectable moiety can be ascertained by quenching activity).

The association of two molecules can be detected by fluorescence resonance energy transfer (FRET). The characteristics of FRET can depend on the FRET pair selected. For example, association of the FRET pair within a FRET radius can permit excitation of a FRET acceptor by a FRET donor. For example, association of a FRET pair within a FRET radius can permit quenching of a fluorophore by a quencher. As such, in some embodiments, for example, in no-wash assays, the detectable moiety comprises a member of a FRET pair. For example, the no wash assay can comprise a first antibody that specifically binds to an HMGB1 isoform of interest, and a second antibody that can simultaneously bind to the HMGB1 isoform, and the first antibody can comprise a first member of a FRET pair while the second antibody can comprise the second member of the fret pair.

Enzyme-substrate pairs can be used to determine the presence or absence of a molecule in an assay. An enzyme and substrate pair can be selected so that, upon processing of the substrate by the enzyme, a signal can be detected, for example a color change or chemiluminescence. In some embodiments, the detectable moiety comprises an enzyme. The presence or absence of the detectable moiety can be determined by the addition of an appropriate substrate. In some embodiments, the detectable moiety comprises a substrate. The presence or absence of the detectable moiety can be determined by the addition of an appropriate enzyme. In some embodiments, the enzyme comprises alkaline phosphatase (AP) or horseradish peroxidase (HRP). In some embodiments, the substrate comprises a chromagen. Exemplary enzyme-chromagen pairs include AP and p-Nitrophenyl Phosphate, Disodium Salt (PNPP), HRP and any of 3,3′,5,5′-Tetramethylbenzidine (TMB), o-Phenylenediamine (OPD), or 2,2′-Azinobis [3-ethylbenzothiazoline-6-sulfonic acid]-diammonium salt (ABTS), and the like. In some embodiments, the substrate comprises a substrate for chemiluminescence. HRP can catalyze the oxidation of luminol, or a variety of other chemiluminscene substrates (often referred to as “ECL” or enhanced chemiluminesce substrates), for example QuantaBlu Fluorogenic Peroxidase Substrate™ substrate (Thermo-Fisher Scientific), NOVEX ECL Chemiluminescent Substrate (Life Technologies), or Clarity™ Western ECL Substrate (BioRad), thus emitting light. As such, in some embodiments, the enzyme-chemiluminescent substrate pair is HRP and luminol or a derivative or luminol.

Raman scattering is a laser-based optical spectroscopy that generates a fingerprint-like vibrational spectrum with features that are much narrower than fluorescence. Raman scattering can be excited using monochromatic far-red or near-IR light, photon energies too low to excite the inherent background fluorescence in biological samples. In surface enhanced Raman spectroscopy (SERS), molecules in very close proximity to nanoscale roughness features on noble metal surfaces (e.g., gold, silver copper) give rise to million- to trillion-fold increases in scattering efficiency compared to normal Raman spectroscopy. In some embodiments, the detectable moiety comprises a SERS tag. A SERs tag can comprise any molecule that provides a Raman signal upon exposure to appropriate irradiation. A number of distinct reporter molecules with strong Raman spectra are known and can be used to create distinct “flavors’ of SERS-active particles to enable multiplexing capabilities (the term “flavors” indicates particles that provide distinct Raman signatures upon irradiation). Examples of molecules that provide strong Raman signals and are suitable for use in SERs tags include, but are not limited to, 4-mercaptopyridine (4-MP); trans-4,4′ bis(pyridyl)ethylene (BPE); quinolinethiol; 4,4′-dipyridyl, 1,4-phenyldiisocyanide; mercaptobenzamidazole; 4-cyanopyridine; 1′,3,3,3′,3 ‘-hexamethylindotricarbocyanine iodide; 3,3’-diethyltiatricarbocyanine; malachite green isothiocyanate; bis-(pyridyl)acetylenes; Bodipy; and isotopes of the foregoing, such as deuterated BPE, deuterated 4,4′-dipyridyl, and deuterated bis-(pyridyl)acetylenes; as well as pyridine, pyridine-d5 (deuterated pyridine), and pyridine-¹⁵N. Various SERS-active particles, SERS-reporter probes and techniques to produce those SERS-active particles and SERS-reporter probes for detection of bioagents, such as nucleic acids, are described, for example, in U.S. Pat. Nos. 6,149,868, 6,514,767, 6,861,263, 7,723,100, and US Patent Publication Nos. 2003/0166297, 2003/0166297, 2007/0259437, 2009/0298197, 2009/0121193, and 2011/0275061, which are herein incorporated by references in their entireties.

In some embodiments, the detectable moiety comprises a nanoparticle. In some embodiments, the nanoparticle comprises at least one noble metal. In some embodiments, the nanoparticle comprises at least one of gold, silver, or copper. In some embodiments, the nanoparticle comprises a metal oxide. In some embodiments, the detectable moiety comprises a quantum dot. In some embodiments, the detectable moiety comprises a radiolabel. For example, the detectable moiety can comprise a gamma-radioactive isotopes of iodine, such as 125-I, attached to tyrosine. Exemplary radioactive substances that can be used as suitable radio labels include, but are not limited to ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac.

In some embodiments, a detectable moiety is directly conjugated to an antibody. For example, the detectable moiety can be covalently linked to an antibody, for example via peptide linker or crosslinker. As such, in some embodiments, an antibody that binds specifically to an HMGB1 isoform of interest, in which the antibody comprises a detectable moiety is provided.

In some embodiments, a detectable moiety is associated with a secondary antibody that binds specifically to an antibody that binds specifically to an HMGB1 isoform of interest.

A secondary antibody can aid in the detection of target antigens by binding to a primary antibody that directly binds to the target molecule of interest, for example HMGB1, through a mechanism that is well known by those skilled in the art. Secondary antibodies increase sensitivity through the signal amplification that occurs as multiple secondary antibodies bind to a single primary antibody.

Secondary antibodies can be generated by immunizing a host animal with an antibody from a different species. Acceptable secondary antibodies in accordance with some embodiments herein include those with specificity against whole IgG (IgG, IgM, IgA, IgD, IgE), and against specific antibody fragments including but not limited to, heavy and light chains, Fragment crystallizable region, fragment antigen binding, F(ab′)2 (heavy and light chain regions forming the antigen-binding domains as well as the hinge region), IgM, Fc5μ (5 connected Fc regions of IgM including the lower portion of the mu heavy chain), M (mu heavy chain), γ (gamma heavy chain), κ (kappa light chain), λ (lambda light chain), alpha heavy chain, and monovalent Fab fragments.

The presence or absence of an antibody comprising a detectable moiety can be detected directly. Accordingly, in some embodiments, an antibody that binds to an HMGB1 isoform of interest itself comprises a detectable moiety. The presence of an antibody that binds to an HMGB1 isoform of interest can also be detected indirectly. Accordingly, in some embodiments, the antibody does not comprise a detectable moiety, but the detectable moiety can later be directly or indirectly associated with the antibody. In some embodiments, a secondary antibody that binds specifically to the antibody that binds to an HMGB1 isoform of interest comprises the detectable moiety. In some embodiments, the antibody that binds to an HMGB1 isoform of interest is biotinyalted, and the detectable moiety comprises avadin.

### Samples

A number of samples can be used in accordance with embodiments herein. As used herein, a “biological sample” contemplates a sample obtained from an organism or from components (e.g., cells) of an organism, including cell cultures. The sample may be of any biological tissue or fluid, for example. Usually, the sample is a biological or a biochemical sample. Frequently the sample will be a “clinical sample” which is a sample derived from a patient. Such samples include, but are not limited to, sputum, cerebrospinal fluid, blood, blood fractions such as serum including fetal serum (e.g., SFC) and plasma, blood cells (e.g., white cells), tissue or fine needle biopsy samples, urine, peritoneal fluid, and pleural fluid, or cells there from. Biological samples may also include sections of tissues such as frozen sections taken for histological purposes. The sample can be, for example, also a physiological sample. In some embodiments, the biological sample comprises serum from a subject. Optionally, the subject can be known to have, or suspected of having mesothelioma, asbestos exposure, or an inflammatory cancer.

As used herein, a “control sample” or “standard” relates to a sample of which the expression level, amount and/or abundance of HMGB1 (or one or more particular isoforms of HMGB1) is known, or has been determined previously. As such, the control sample may be derived from a “healthy” person, i.e., a person diagnosed previously as not suffering or predisposed from the pathological condition(s) at issue (e.g. mesothelioma or inflammatory cancer). Alternatively, the control sample may be derived from a “diseased” person, i.e. a person diagnosed previously as suffering or predisposed from a disease other than mesothelioma or inflammatory cancer, or a person diagnosed as having mesothelioma or inflammatory cancer at a particular stage. A control sample or standard can be spiked with a known amount or level of molecules, for example a known amount or level of one or more of hyper-acetylated HMGB1, hypo-acetylated HMGB1, disulfide HMGB1, fully-reduced HMGB1, hyper-acetylated disulfide HMGB1, hypo-acetylated disulfide HMGB1, hyper-acetylated fully-reduced HMGB1, or hypo-acetylated fully-reduced HMGB1. In a further alternative, the control sample may be synthetic, i.e. not derived from a person, but comprising a known amount of molecules. In some embodiments, a predetermined level is based upon a control or standard, for example a control known to be characteristic of a subject that has malignant mesothelioma or inflammatory cancer.

### Immunological Assays

Immunological assays (also referred to herein as “immunoassays”), including radioimmunoassays and enzyme-linked immunoassays, are useful in the methods of several embodiments provided herein. Furthermore, a variety of monoclonal and polyclonal antibodies that bind to HMGB1 isoforms as described herein are useful in immunoassays. A variety of immunoassay formats, including competitive and non-competitive immunoassay formats, antigen capture assays and two antibody sandwich assays also are useful in accordance with the methods and kits of embodiments herein (for a summary of various immunoassays, see Self and Cook, Curr. Opin. Biotechnol. 7:60-65 (1996), which is hereby incorporated by reference in its entirety). Exemplary immunoassays that are suitable in accordance with some embodiments herein include immunoassays, lateral flow assays, no-wash assays, sandwich immunoassays, competition immunoassays, ELISA, immunoblot assays, flow cytometry, immunohistochemistry, surface plasmon resonance, western blots, immunoblots, and the like.

### ELISA

Enzyme-linked immunosorbent assays (ELISAs) can be useful in accordance with some embodiments herein.

In some embodiments, ELISA comprises a sandwich ELISA. Sandwich ELISA is known to the skilled artisan. In a sandwich ELISA, a capture antibody that binds to the molecule of interest, for example HMGB1, is provided. The capture antibody can be immobilized on a substrate, for example a membrane, a bead, or a well of plate such a microtiter plate. A primary antibody that binds to the molecule of interest can be provided. The presence of bound primary antibody can be detected, for example directly (e.g. if the primary antibody comprises a detectable moiety) or indirectly (e.g. by contacting the assay environment with a secondary antibody comprising the detectable moiety).

It will be appreciated by the skilled artisan that at least one of the primary antibody or capture antibody binds specifically to a specific isoform of HMGB1 (e.g. hyper-acetylated HMGB1, hypo-acetylated HMGB1, disulfide HMGB1, fully-reduced HMGB1, hyper-acetylated disulfide HMGB1, hypo-acetylated disulfide HMGB1, hyper-acetylated fully-reduced HMGB1, or hypo-acetylated fully-reduced HMGB1).

Optionally, the capture antibody can comprise a monoclonal antibody that binds specifically to an epitope characteristic of the HMGB1 isoform of interest, and the primary antibody can comprise a polyclonal antibody that binds generally to HMGB1.

Optionally, the capture antibody can comprise a polyclonal antibody that binds generally to HMGB1, and the primary antibody can comprise a monoclonal antibody that binds specifically to an epitope characteristic of the HMGB1 isoform of interest

In some embodiments, ELISA comprises competitive ELISA. Known to those skilled in the art, competitive ELISA is a competitive binding process between any molecule of interest in a sample, for example HMGB1, and a “competing molecule” that competes for binding with the molecule of interest (for example the molecule of interest itself, or an analog thereof). In competitive ELISA, the unlabeled primary antibody is incubated with a sample that possibly comprises the molecule of interest, which is then added to a reaction environment comprising the competing molecule. Optionally, the competing molecule can be immobilized on a substrate, for example coated on the surface of a well in a multi-well plate such as a 96-well plate. Any unbound primary antibody is washed away, and a secondary antibody that is specific to the primary antibody and conjugated with a detectable moiety, for example an enzyme, is added. The detectable moiety can be detected, for example, if the detectable moiety comprises an enzyme, by adding an appropriate substrate for the enzyme so as to produce a chromogenic or fluorescent signal. The higher the concentration of the molecule of interest in the sample, for example an HMGB1 isoform of interest, the weaker the eventual signal from the detectable moiety. It will be appreciated that if the molecule of interest comprises a particular HMGB1 isoform (as in accordance with the methods and kits of various embodiments herein), the primary antibody can bind specifically to a that isoform of HMGB1, for example, hyper-acetylated HMGB1, hypo-acetylated HMGB1, disulfide HMGB1, fully-reduced HMGB1, hyper-acetylated disulfide HMGB1, hypo-acetylated disulfide HMGB1, hyper-acetylated fully-reduced HMGB1, or hypo-acetylated fully-reduced HMGB1.

### Competition Immunoassays

In some embodiments, competition immunoassays (sometimes also referred to a “competitive immunoassays” and the like) are provided. Competition immunoassays can comprise providing a quantity of binding agent and a quantity of target molecules (e.g. biomarker) labeled with a detectable moiety. In the presence of unlabeled target molecule, for example the presence of biomarker in a sample, the unlabeled target molecule can compete with the labeled target molecule for binding to the binding agent. Accordingly, the greater the quantity unlabeled target molecule that is present in the sample, the greater the quantity of labeled target molecule that gets displaced. As such, a lower amount of label (detectable moiety) associated with the binding agent can indicate a higher amount of unlabeled target molecule in a sample. By way of example, competition immunoassays can be used for The Enzyme-Linked Immunosorbent Assay (ELISA), lateral flow systems, no-wash assays, and the like.

### Lateral Flow Systems

In some embodiments, lateral flow systems are provided. The lateral flow system can comprise a single device of platform on which any of a plurality of different types of assay pellets can be used. The lateral flow system can comprise a substrate. The substrate can be configured for the capillary flow of an analyte over and/or through the substrate. The substrate can be configured to bind specifically to a binding partner that comprises at least one of a biomarker, binding agent, or complex comprising a biomarker and binding agent. Accordingly, when the substrate is contacted with a fluid, complexes of biomarker, binding agent, and detectable moiety, if present in the fluid, can be immobilized on the substrate, while unbound binding agent and/or detectable moiety are not immobilized.

In some embodiments, a single surface of the substrate is configured to bind to each of the binding agents of the assay pellets. The detectable moiety of each different assay pellet can be different.

In some embodiments, a lateral flow assay cartridge comprising a single assay pellet, and a substrate configured to bind specifically to the binding agent of the assay pellet or associated biomarker is provided. In some embodiments, a plurality of lateral flow assay cartridges is provided, each of which comprises an assay pellet for a different biomarker. In some embodiments, a reservoir configured for fluid communication with the substrates of two or more lateral flow cartridges is provided.

### No-Wash Assays

In some embodiments, no-wash assays are provided. A no-wash assay can detect the presence or absence of a molecule of interest through the detection of a signal (or the absence of a signal) indicating the association of two different detectable moieties. In some embodiments, the two different detectable moieties are a FRET pair. In some embodiments, the FRET pair comprises a donor moiety and an acceptor moiety. In some embodiments, the two different detectable moieties are a fluorophore quencher pair. The no-wash system can include a first antigen binding molecule (for example an antibody) and a second antigen binding molecule, each of which bind to the same target at a different epitope. As such, in some embodiments, the first antigen binding molecule and the second antigen binding molecule can be bound to the same target at the same time. The signal (or absence of signal) produced by the association of the detectable moiety of the first antigen binding molecule and that of the second antigen binding molecule can indicate that both antigen binding molecules have bound to the target. By way of example, in accordance with some embodiments herein, a no-wash assay (or kit therefor) comprises a first antibody that binds to an HMGB1 isoform of interest, for example a monoclonal antibody that specifically binds to an epitope characteristic of the HMGB1 isoform of interest. The no-way assay (or kit) can further comprise a second antibody that also binds to the HMGB1 isoform of interest, but does not compete for binding with the first antibody. As such, the second antibody can optionally bind to a second epitope characteristic of the HMGB1 isoform of interest (for example a second monoclonal antibody that binds to a different epitope than the first antibody), or can optionally bind generally to HMGB1 (for example a polyclonal antibody that binds to HMGB1).

In some embodiments, a no-wash assay includes a plurality of different detection assays in a single reaction environment. In some embodiments a first binding agent-detectable moiety pair each comprising a different member of a first FRET pair, and a second binding agent-detectable moiety pair each comprising a different member of a second FRET pair that is different from the first FRET pair are assessed in the same reaction environment. In some embodiments, at least two different FRET pairs, for example 2, 3, 4, 5, 6, 7, 8, 9, or 10, FRET pairs are assessed in the same reaction environment. As such, a multiplex no-wash assay can be performed.

In some embodiments, the reaction environment of a no-wash assay comprises a well in a multi-well format plate, a test tube, a cuvette, a flask, or the like. In some embodiments, the reaction environment of a no-wash system is configured for detection by an electromagnetic radiation detector. As such, in some embodiments, at least one surface of the reaction environment is penetrable to electromagnetic radiation. In some embodiments, the electromagnetic radiation has a wavelength in the visible spectrum. In some embodiments, the electromagnetic radiation has a wavelength in a fluorescent excitation and emission spectrum.

### Mass Spectrometry

Mass spectrometry can be used in methods in accordance with some embodiments herein to detect isoforms of HMGB1.

Example isoforms of HMGB1 include a 24.585 KDa isoform of HMGB1 (e.g. hypo-acetylated disulfide HMGB1); a 24.587 KDa isoform of HMGB1 (e.g. hypo-acetylated fully reduced HMGB1); a 25.467 KDa isoform of HMGB1 (e.g. hyper-acetylated disulfide HMGB1); and a 25.469 KDa isoform of HMGB1 (e.g. hyper-acetylated fully reduced HMGB1). Mass spectrometry is a technique that helps measure the amount and type of chemicals present in a sample. It analyzes the mass-to-charge ratio and presence of gas-phase ions. Known to those skilled in the art, the mass spectrum can be used to determine the elements or isotopes in a sample, the masses of particles of molecules, and thus the structures of molecules can be determined. Mass spectrometry can be used in accordance with embodiments herein to determine the masses and structures of various isoforms of HMGB1.

In some embodiments, mass spectrometry/mass spectrometry (MS/MS) is used to determine the presence and/or levels of one or more isoform of HMGB1. In MS/MS a particular characteristic peak of a mass spec profile is further analyzed (e.g. sequenced). This is also known as Triple Quadruple Mass Spectrometry.

In some embodiments, Electrospray Ionization Liquid Chromatography Mass Spectrometry (ESI-LC-MS) is used to determine the presence and/or levels of one or more isoform of HMGB1. ESI-LC-MS is well known to those skilled in the art. In ESI-LC-MS, molecular ions, as well as structural information of the molecules can be observed, and the solution-phase information can also be retained into the gas-phase if needed.

In some embodiments, Matrix-assisted Laser desorption/ionization (MALDI) is used to determine the presence and/or levels of one or more isoform of HMGB1. MALDI is a soft ionization technique used in mass spectrometry, and can allows the analysis of proteins and other biomolecules, which tend to be fragile and fragmented when ionized by more conventional ionization methods.

### Method of Determining an HMGB1 Isoform Signature of a Subject

HMGB1 isoform “signatures” include the presence or level of one or more isoforms of HMGB1 (or presence or level of antibodies bound to one more isoforms of HMGB1) in a sample in the subject relative to a predetermined threshold. For example, the signal can include the presence or level of two or more, three or more, four or more, five or more, or six or more HMGB1 isoforms. The signature can provide information about a condition of the subject. For example, an HMGB1 isoform signature for a subject can comprise hypo-acetylated HMGB1 determined to be above or below a predetermined level. For example, an HMGB1 isoform signature for a subject can comprise hypo-acetylated HMGB1 determined to be above or below a first predetermined level and hyper-acetylated HMGB1 as determined to be present or absent, or above or below a second predetermined level. For example, an HMGB1 isoform signature can comprise disulfide HMGB1 determined to be present or absent, or above or below a predetermined level. For example, and HMGB1 isoform signature can comprise disulfide HMGB1 determined to be present or absent, or above or below a first predetermined level, and fully reduced HMGB1 determined to be above or below a second predetermined level. HMGB1 signatures in accordance with some embodiments herein can be determined by a variety of methods, for example by contacting a biological sample with one or more antibodies that bind specifically to particular HMGB1 isoforms as described herein, or by mass spectrometry. Optionally, the HMGB1 isoform signature comprises a binding pattern of one or more antibodies specific for HMGB1 isoforms, for example a spatial and/or temporal pattern of binding.

In some embodiments, the HMGB1 signature is compared to a suitable negative control, for example a healthy patient, and/or for example, from a patient with pleural effusions. As shown in Tables 3.1-3.12 and 4.1-4.4, smoking status was observed to vary with total HMGB1 levels. Accordingly, in some embodiments, for example, embodiments in which total HMGB1 is assessed, a suitable negative control comprises a sample of an individual with the same smoking status (e.g. smoker or non-smoker) as the subject being assessed. In some embodiments, for example embodiments in which total HMGB1 is assessed, there is a first predetermined level for non-smokers, and a second predetermined level for smokers. For example, the predetermined level of total HMGB1 for non-smokers can be at least about 9 ng/ml, for example, at least about 9 ng/ml, 10, 11, 12, 13, 14, 15, 16, 17, 18, 19, or 20 ng/ml. For example, the predetermined level of total HMGB1 for smokers can be at least about 19 ng/ml, for example, at least about 19 ng/ml, 20, 21, 22, 23, 24, 25, 26, 27, 28, 29, or 30 ng/ml.

In accordance with some embodiments, methods of determining an HMGB1 isoform signature of a subject are provided. Optionally, the subject is suspected of having mesothelioma or an inflammatory condition. The method can include contacting a biological sample of the subject with an antibody that binds to an HMGB1 isoform of interest, for example hyper-acetylated HMGB1, disulfide HMGB1 (HMGB1C23-C45), hypo-acetylated HMGB1, fully reduced HMGB1, hyper-acetylated disulfide HMGB1, hyper-acetylated fully reduced HMGB1, hypo-acetylated disulfide HMGB1, or hypo-acetylated fully-reduced HMGB1. The method can include detecting a level of the antibody bound to HMGB1 in the biological sample. Bound levels of antibody to the sample can identify a signature characteristic of a condition in the subject. Optionally, the method can include detecting a level of one or more HMGB1 isoforms in the biological sample. Levels of HMGB1 isoforms in the sample can identify a signature characteristic of a condition in the subject

Optionally, the signature is determined in accordance with the example signatures of Table 1. Example signatures are shown across rows, with optional parameters of the signature shown as “-”

TABLE 1
Example signatures

| | Hyper- | Hypo- | | Fully | |
|---|---|---|---|---|---|
| Associated | acetylated | acetylated | Disulfide | Reduced | Total |
| Condition | HMGB1 | HMGB1 | HMGB1 | HMGB1 | HMGB1 |
| Healthy | — | <4 ng/ml | — | — | — |
| Individual | | | | | |
| Healthy | — | — | — | Not | — |
| Individual | | | | detected | |
| Healthy | — | <4 ng/ml | — | Not | — |
| Individual | | | | detected | |
| Asbestos-Exposed | — | — | — | — | >3.00 ng/ml |
| or MM | | | | | |
| Asbestos-Exposed | — | — | — | — | >3.05 ng/ml |
| or MM | | | | | |
| Asbestos-Exposed | — | >4 ng/ml | — | — | |
| or MM | | | | | |
| Asbestos-Exposed | — | — | — | — | >3.05 ng/ml |
| | | | | | and |
| | | | | | <15.75 ng/ml |
| Asbestos-Exposed | <10 ng/ml | >4 ng/ml | — | — | — |
| Asbestos-Exposed | Not | >4 ng/ml | — | — | — |
| | detected | | | | |
| Asbestos-Exposed | — | — | Not detected | >lower | — |
| | | | | detection | |
| | | | | limit | |
| Asbestos-Exposed | <10 ng/ml | — | — | >lower | — |
| | | | | detection | |
| | | | | limit | |
| Asbestos-Exposed | Not | — | — | >lower | — |
| | detected | | | detection | |
| | | | | limit | |
| Asbestos-Exposed | — | >4 ng/ml | Not detected | — | — |
| Asbestos-Exposed | present, but | >4 ng/ml | — | — | — |
| | <2 ng/ml | | | | |
| Asbestos-Exposed | present, but | — | — | >lower | — |
| | <2 ng/ml | | | detection | |
| | | | | limit | |
| MM or | — | — | — | — | >15.75 ng/ml |
| inflammatory | | | | | |
| cancer | | | | | |
| MM or | >2 ng/ml | — | — | — | >15.75 ng/ml |
| inflammatory | | | | | |
| cancer | | | | | |
| MM or | — | — | >5-fold over | — | >15.75 ng/ml |
| inflammatory | | | lower | | |
| cancer | | | detection | | |
| MM or | >10 ng/ml | — | — | — | — |
| inflammatory | | | | | |
| cancer | | | | | |
| MM or | >2 ng/ml | — | — | — | — |
| inflammatory | | | | | |
| cancer | | | | | |
| MM or | — | — | >5-fold over | — | — |
| inflammatory | | | lower | | |
| cancer | | | detection | | |
| | | | limit | | |
| MM or | — | — | >5-fold over | >lower | — |
| inflammatory | | | lower | detection | |
| cancer | | | detection | limit | |
| | | | limit | | |
| MM or | — | >4 ng/ml | >5-fold over | — | — |
| inflammatory | | | lower | | |
| cancer | | | detection | | |
| | | | limit | | |
| MM or | >10 ng/ml | — | >5-fold over | >lower | — |
| inflammatory | | | lower | detection | |
| cancer | | | detection | limit | |
| | | | limit | | |
| MM or | >10 ng/ml | >4 ng/ml | >5-fold over | >lower | — |
| inflammatory | | | lower | detection | |
| cancer | | | detection | limit- | |
| | | | limit | | |
| MM or | >10 ng/ml | >4 ng/ml | — | >lower | — |
| inflammatory | | | | detection | |
| cancer | | | | limit | |
| MM or | >2 ng/ml | — | >5-fold over | >lower | — |
| inflammatory | | | lower | detection | |
| cancer | | | detection | limit | |
| | | | limit | | |
| MM or | >2 ng/ml | >4 ng/ml | >5-fold over | >lower | — |
| inflammatory | | | lower | detection | |
| cancer | | | detection | limit | |
| | | | limit | | |
| MM or | >2 ng/ml | >4 ng/ml | — | >lower | — |
| inflammatory | | | | detection | |
| cancer | | | | limit | |
| MM or | — | >4 ng/ml | >5-fold over | >lower | — |
| inflammatory | | | lower | detection | |
| cancer | | | detection | limit | |
| | | | limit | | |
| MM or | — | — | — | >10-fold | — |
| inflammatory | | | | over lower | |
| cancer | | | | detection | |
| | | | | limit | |
| MM or | — | — | >5-fold over | >10-fold | — |
| inflammatory | | | lower | over lower | |
| cancer | | | detection | detection | |
| | | | limit | limit | |
| MM or | — | >4 ng/ml | — | >10-fold | — |
| inflammatory | | | | over lower | |
| cancer | | | | detection | |
| | | | | limit | |
| MM or | >10 ng/ml | — | — | >10-fold | — |
| inflammatory | | | | over lower | |
| cancer | | | | detection | |
| | | | | limit | |
| MM or | — | >4 ng/ml | >5-fold over | >10-fold | — |
| inflammatory | | | lower | over lower | |
| cancer | | | detection | detection | |
| | | | limit | limit | |
| MM or | >10 ng/ml | >4 ng/ml | | >10-fold | — |
| inflammatory | | | | over lower | |
| cancer | | | | detection | |
| | | | | limit | |
| MM or | >10 ng/ml | | >5-fold over | >10-fold | — |
| inflammatory | | | lower | over lower | |
| cancer | | | detection | detection | |
| | | | limit | limit | |
| MM or | Present | — | — | — | — |
| inflammatory | | | | | |
| cancer | | | | | |
| MM or | — | — | Present | — | — |
| inflammatory | | | | | |
| cancer | | | | | |
| MM or | Present | — | Present | — | — |
| inflammatory | | | | | |
| cancer | | | | | |
| MM (but not | — | — | — | — | >11.35 ng/ml |
| benign or non- | | | | | |
| MM pleural | | | | | |
| effusion) | | | | | |
| MM (but not | >9.70 ng/ml | — | — | — | — |
| benign or non- | | | | | |
| MM pleural | | | | | |
| effusion) | | | | | |
| MM (but not | >9.70 ng/ml | — | — | — | >11.35 ng/ml |
| benign or non- | | | | | |
| MM pleural | | | | | |
| effusion) | | | | | |

For HMGB1 isoform signatures, the presence or level relative to a predetermined level of one or more isoforms of HMGB1 in a sample in a subject can be useful to indicate the condition of a subject. An HMGB1 isoform signature can include a presence, level, or level of antibody bound to one or more of (a) hyper-acetylated HMGB1, (b) disulfide HMGB1 (HMGB1C23-C45), (c) hypo-acetylated HMGB1, (d) fully reduced HMGB1, (e) hyper-acetylated disulfide HMGB1, (f) hyper-acetylated fully reduced HMGB1, (g) hypo-acetylated disulfide HMGB1, and (h) hypo-acetylated fully-reduced HMGB1. Optionally, an HMGB1 isoform signature can comprise two or more of (a)-(h), for example two, three, four, five six, seven, or eight of (a)-(h), for example (a) and (b), (a) and (c), (a) and (d), (a) and (e), (a) and (f), (a) and (g), (a) and (h), (b) and (c), (b) and (d), (b) and (e), (b) and (f), (b) and (g), (b) and (h), (c) and (d), (c) and (e), (c) and (f), (c) and (g), (c) and (h), (d) and (e), (d) and (f), (d) and (g), (d) and (h), (e) and (f), (e) and (g), or (e) and (h).

Optionally, the levels of any of (a)-(h) in an HMGB1 isoform signature are compared to a predetermined level, in which the level in the subject below the predetermined level indicates an HMGB1 isoform signature characteristic of a healthy subject. For example, levels of (c) hypo-acetylated HMGB1 below the predetermined level indicates an HMGB1 isoform signature characteristic of a healthy subject. For example, an absence of (d) fully reduced HMGB1, (f) hyper-acetylated fully reduced HMGB1, and (h) hypo-acetylated fully-reduced HMGB1 indicate an HMGB1 isoform signature characteristic of a healthy subject (Table 1). Optionally, an HMGB1 isoform signature can differentiate a malignant mesothelioma patient from a patient with pleural effusions, for example based on the levels of any one of (a)-(h).

Optionally, the levels of any of (a)-(h) in a subject are compared to a predetermined level, in which the level in the subject above the predetermined level indicates an HMGB1 isoform signature characteristic of a subject that has at least one of asbestos exposure, malignant mesothelioma or an inflammatory cancer. For example, the levels of (c) hypo-acetylated HMGB1 above a first predetermined level and binding of (a) hyper-acetylated HMGB1 above a second predetermined level can identify an HMGB1 isoform signature characteristic of a subject that has malignant mesothelioma or an inflammatory cancer. Optionally, the level of binding of (c) hypo-acetylated HMGB1 above a first predetermined level and binding of (a) hyper-acetylated below a second predetermined level identifies an HMGB1 isoform signature characteristic of a subject that has asbestos exposure, but not malignant mesothelioma. For example, the level of binding of (d) fully reduced HMGB1 above a first predetermined level and binding of (b) disulfide HMGB1 (HMGB1C23-C45) above a second predetermined level can identify an HMGB1 isoform characteristic of a subject that has malignant mesothelioma or an inflammatory cancer. Optionally, the level of binding of (d) fully reduced HMGB1 above a first predetermined level and binding of (b) disulfide HMGB1 (HMGB1C23-C45) below a second predetermined level identifies an HMGB1 isoform signature characteristic of a subject that has asbestos exposure, but not malignant mesothelioma. For example, the level of binding of (c) hypo-acetylated HMGB1 above a first predetermined level and binding of (a) hyper-acetylated HMGB1 above a second predetermined level can identify an HMGB1 isoform signature characteristic of a subject that has an inflammatory cancer. Furthermore, optionally the level of binding of (d) fully reduced HMGB1 above a first predetermined level and binding of (b) disulfide HMGB1 (HMGB1C23-C45) above a second predetermined level can identify an HMGB1 isoform signature characteristic of a subject that has an inflammatory cancer. Inflammatory cancers that can be detected, detected, or diagnosed by the methods in accordance with these methods include, but are not limited to, mesothelioma, colorectal cancer, pancreatic cancer, breast cancer, liver cancer, and the like.

Optionally, the antibody that binds specifically to hyper-acetylated HMGB1 binds specifically to an acetylated lysine in NLS1 or an acetylated lysine in NLS2 of HMGB1. Optionally, the antibody that binds specifically hyper-acetylated HMGB1 binds specifically to acetylated K30, acetylated K43, acetylated K90, and/or acetylated K141 of HMGB1. Optionally, the antibody that binds specifically to reduced HMGB1 binds specifically to reduced C23, reduced C45, and/or reduced C106 of HMGB1. Optionally, the antibody that binds specifically to disulfide HMGB1 binds specifically to an epitope comprising C23 bonded to C45 by a disulfide bond.

Optionally, the predetermined level of (a) hyper-acetylated HMGB1 is at least about 0.1 ng/ml (or an antibody bound to at least about 0.1 ng/ml of hyper-acetylated HMGB1), for example at least about 0.1 ng/ml, 0.3, 0.4, 0.5, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 13, 14, 15, 16, 17, 18, 19, 20, 21, 22, 23, 34, 25, 30, 35, 40, 45, 50, 60, 70, 80, 90, 100, 150, or 200 ng/mL of hyper-acetylated HMGB1. Optionally, the subject is identified as having MM or an inflammatory cancer if the level falls within a range any two of the listed values, for example, about 3-23 ng/ml of hyper-acetylated HMGB1, for example about, 3-21 ng/ml, 3-19 ng/ml, 3-17 ng/ml, 3-15 ng/ml, 3-13 ng/ml, 3-11 ng/ml, 3-9 ng/ml, 3-7 ng/ml, 3-5 ng/ml, 5-23 ng/ml, 7-23 ng/ml, 9-23 ng/ml, 11-23 ng/ml, 13-23 ng/ml, 15-23 ng/ml, 17-23 ng/ml, 19-23 ng/ml, 21-23 ng/ml, 5-21 ng/ml, 7-19 ng/ml, 5-13 ng/ml, 7-13 ng/ml, 9-13 ng/ml. Optionally, a presence or absence of hyper-acetylated HMGB1 is determined.

Optionally, the predetermined level of (b) disulfide HMGB1 is at least about 0.1 ng/ml (or an antibody bound to at least about 0.1 ng/ml of disulfide HMGB1), for example at least about 0.1 ng/ml, 0.2, 0.3, 0.4, 0.5, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 13, 14, 15, 16, 17, 18, 19, 20, 21, 22, 23, 34, 25, 30, 35, 40, 45, 50, 60, 70, 80, 90, 100, 150, or 200 ng/ml of disulfide HMGB1. Optionally, the predetermined level of (b) disulfide HMGB1 is at least about 5 ng/ml. Optionally, the subject is identified as having MM or an inflammatory cancer if the level falls within a range of any two of the listed values, for example, about 3-23 ng/ml of disulfide HMGB1, for example about, 3-21 ng/ml, 3-19 ng/ml, 3-17 ng/ml, 3-15 ng/ml, 3-13 ng/ml, 3-11 ng/ml, 3-9 ng/ml, 3-7 ng/ml, 3-5 ng/ml, 5-23 ng/ml, 7-23 ng/ml, 9-23 ng/ml, 11-23 ng/ml, 13-23 ng/ml, 15-23 ng/ml, 17-23 ng/ml, 19-23 ng/ml, 21-23 ng/ml, 5-21 ng/ml, 7-19 ng/ml, 5-13 ng/ml, 7-13 ng/ml, 9-13 ng/ml. Optionally, the predetermined level of (b) disulfide HMGB1 represents a fold increase (FI) over the lower limit of detection (LLD) for the antibody. In some embodiments, the predetermined level of (c) disulfide HMGB1 comprises a FI of at least about 0.5 FI over LLD, for example, about 0.5 FI over LLD, 0.7, 1, 1.5, 2, 2.5, 3, 3.5, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 400, 500, 600, 700, 800, 900, 1000, 2000, 5000, or 10,000 FI over LLD, including ranges between any two of the listed values. In some embodiments, an individual is identified as having MM or an inflammatory cancer when the level of antibody bound to disulfide HMGB1 is within about 3-41 Fl over LLD, 3-39 Fl over LLD, 3-37 Fl over LLD, 3-35 Fl over LLD, 3-33 Fl over LLD, 3-31 Fl over LLD, 3-29 Fl over LLD, 3-27 Fl over LLD, 3-25 Fl over LLD, 3-23 Fl over LLD, 5-21 Fl over LLD, 5-19 Fl over LLD, 7-17 Fl over LLD, 9-15 Fl over LLD, 11-13 Fl over LLD, 7-31 Fl over LLD, 9-29 Fl over LLD, 11-27 Fl over LLD, 7-33 Fl over LLD, 9-27 Fl over LLD, 9-25 Fl over LLD. Optionally, a presence or absence of disulfide HMGB1 is determined.

Optionally, the predetermined level of (c) hypo-acetylated HMGB1 is at least about 0.1 ng/ml (or an antibody bound to at least about 0.1 ng/ml of hypo-acetylated HMGB1), for example at least about 0.1 ng/ml, 0.3, 0.4, 0.5, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 13, 14, 15, 16, 17, 18, 19, 20, 21, 22, 23, 34, 25, 30, 35, 40, 45, 50, 60, 70, 80, 90, 100, 150, or 200 ng/ml of hypo-acetylated HMGB1. Optionally, the subject is identified as having MM or asbestos exposure or an inflammatory cancer if the level falls within a range any two of the listed values, for example, about 3-23 ng/ml of hypo-acetylated HMGB1, for example about, 3-21 ng/ml, 3-19 ng/ml, 3-17 ng/ml, 3-15 ng/ml, 3-13 ng/ml, 3-11 ng/ml, 3-9 ng/ml, 3-7 ng/ml, 3-5 ng/ml, 5-23 ng/ml, 7-23 ng/ml, 9-23 ng/ml, 11-23 ng/ml, 13-23 ng/ml, 15-23 ng/ml, 17-23 ng/ml, 19-23 ng/ml, 21-23 ng/ml, 5-21 ng/ml, 7-19 ng/ml, 5-13 ng/ml, 7-13 ng/ml, 9-13 ng/ml. In some embodiments, the subject is determined to not have MM or inflammatory cancer or asbestos exposure if the level of hypo-acetylated HMGB1 falls below the predetermined level.

Optionally, the predetermined level of (d) fully reduced HMGB1 is at least about 0.1 ng/ml (or an antibody bound to at least about 0.1 ng/ml of fully reduced HMGB1), for example at least about 0.1 ng/ml, 0.3, 0.4, 0.5, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 13, 14, 15, 16, 17, 18, 19, 20, 21, 22, 23, 34, 25, 30, 35, 40, 45, 50, 60, 70, 80, 90, 100, 150, or 200 ng/ml of fully reduced HMGB1. Optionally, the predetermined level of (d) fully reduced HMGB1 is at least about 7 ng/ml (Optionally, the subject is identified as having MM or an inflammatory cancer if the level falls within a range any two of the listed values, for example, about 3-23 ng/ml of disulfide HMGB1, for example about, 3-21 ng/ml, 3-19 ng/ml, 3-17 ng/ml, 3-15 ng/ml, 3-13 ng/ml, 3-11 ng/ml, 3-9 ng/ml, 3-7 ng/ml, 3-5 ng/ml, 5-23 ng/ml, 7-23 ng/ml, 9-23 ng/ml, 11-23 ng/ml, 13-23 ng/ml, 15-23 ng/ml, 17-23 ng/ml, 19-23 ng/ml, 21-23 ng/ml, 5-21 ng/ml, 7-19 ng/ml, 5-13 ng/ml, 7-13 ng/ml, 9-13 ng/ml. Optionally, the predetermined level of (d) fully reduced HMGB1 represents a fold increase (FI) over the lower limit of detection (LLD) for the antibody. In some embodiments, the predetermined level of (d) fully reduced HMGB1 comprises a FI of at least about 0.5 FI over LLD, for example, about 0.5 FI over LLD, 0.7, 1, 1.5, 2, 2.5, 3, 3.5, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 400, 500, 600, 700, 800, 900, 1000, 2000, 5000, or 10,000 FI over LLD, including ranges between any two of the listed values. In some embodiments, an individual is identified as having MM or an inflammatory cancer when the level of antibody bound to fully reduced HMGB1 is within about 3-41 Fl over LLD, 3-39 Fl over LLD, 3-37 Fl over LLD, 3-35 Fl over LLD, 3-33 Fl over LLD, 3-31 Fl over LLD, 3-29 Fl over LLD, 3-27 Fl over LLD, 3-25 Fl over LLD, 3-23 Fl over LLD, 5-21 Fl over LLD, 5-19 Fl over LLD, 7-17 Fl over LLD, 9-15 Fl over LLD, 11-13 Fl over LLD, 7-31 Fl over LLD, 9-29 Fl over LLD, 11-27 Fl over LLD, 7-33 Fl over LLD, 9-27 Fl over LLD, 9-25 Fl over LLD.

### Methods of Diagnosing or Identifying Malignant Mesothelioma in a Subject

It is contemplated that levels and presence of some HMGB1 isoforms can be useful for identifying or diagnosing mesothelioma. In accordance with some embodiments herein, methods of diagnosing or identifying malignant mesothelioma in a subject are provided. The method can comprise providing a biological sample of the subject, contacting the biological sample with at least one antibody that binds to an HMGB1 isoform of interest. Optionally, the method comprises contacting the biological sample with one or more of: an antibody that binds specifically to hyper-acetylated HMGB1, an antibody that binds specifically to disulfide HMGB1 (HMGB1C23-C45), an antibody that binds specifically to hypo-acetylated HMGB1, or an antibody that binds specifically to fully reduced HMGB1. The method can comprise detecting a level of the antibody bound to HMGB1 in the sample. The method can comprise comparing the level of bound antibody to a predetermined level. The method can comprise diagnosing the subject as having malignant mesothelioma when the level of antibody bound to HMGB1 is greater than the predetermined level. Optionally, the method can comprise detecting a level of HMGB1 in the sample. The method can comprise diagnosing the subject as having malignant mesothelioma when the level of HMGB1 isoform is greater than the predetermined level.

Optionally, the biological sample is contacted with two, three, or four of: of antibody that binds specifically to hyper-acetylated HMGB1, an antibody that binds specifically to disulfide HMGB1 (HMGB1C23-C45), an antibody that binds specifically to hypo-acetylated HMGB1, or an antibody that binds specifically to fully reduced HMGB1. Optionally, the biological sample is contacted with an antibody that binds specifically to hyper-acetylated HMGB1 and an antibody that binds specifically to disulfide HMGB1 (HMGB1C23-C45). Optionally, the biological sample is contacted with an antibody that binds specifically to hyper-acetylated HMGB1 and an antibody that binds specifically to hypo-acetylated HMGB1. Optionally, the biological sample is contacted with an antibody that binds specifically to hyper-acetylated HMGB1 and an antibody that binds specifically to fully reduced HMGB1. Optionally, the biological sample is contacted with an antibody that binds specifically to disulfide HMGB1 (HMGB1C23-C45) and an antibody that binds specifically to hypo-acetylated HMGB1. Optionally, the biological sample is contacted with an antibody that binds specifically to disulfide HMGB1 (HMGB1C23-C45) and an antibody that binds specifically to fully reduced HMGB1. Optionally, the biological sample is contacted with an antibody that binds specifically to hypo-acetylated HMGB1 and an antibody that binds specifically to fully reduced HMGB1.

Optionally, the biological sample is contacted with an antibody that binds specifically to hyper-acetylated HMGB1, an antibody that binds specifically to disulfide HMGB1 (HMGB1C23-C45), and an antibody that binds specifically to hypo-acetylated HMGB1. Optionally, the biological sample is contacted with an antibody that binds specifically to hyper-acetylated HMGB1, an antibody that binds specifically to disulfide HMGB1 (HMGB1C23-C45), and an antibody that binds specifically to fully reduced HMGB1. Optionally, the biological sample is contacted with an antibody that binds specifically to hyper-acetylated HMGB1, and an antibody that binds specifically to hypo-acetylated HMGB1, an antibody that binds specifically to fully reduced HMGB1. Optionally, the biological sample is contacted with an antibody that binds specifically to disulfide HMGB1 (HMGB1C23-C45), an antibody that binds specifically to hypo-acetylated HMGB1, and an antibody that binds specifically to fully reduced HMGB1.

Optionally, the subject is identified as having MM when the level of hyper-acetylated HMGB1 (or level of antibody bound to hyper-acetylated HMGB1) in the sample is greater than a level of hyper-acetylated HMGB1 (or the antibody bound to hyper-acetylated HMGB1) of about 0.1 ng/ml of hyper-acetylated HMGB1, for example greater than about 0.1, 0.2, 0.3, 0.4, 0.5, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 13, 14, 15, 16, 17, 18, 19, 20, 21, 22, 23, 24, 25, 30, 35, 40, 45, 50, 60, 70, 80, 90, 100, 150, or 200 ng/mL, including ranges between any two of the listed values. Optionally, the subject is identified as having MM if the level falls within a range any two of the listed values, for example, 3-23 ng/ml, 3-21 ng/ml, 3-19 ng/ml, 3-17 ng/ml, 3-15 ng/ml, 3-13 ng/ml, 3-11 ng/ml, 3-9 ng/ml, 3-7 ng/ml, 3-5 ng/ml, 5-23 ng/ml, 7-23 ng/ml, 9-23 ng/ml, 11-23 ng/ml, 13-23 ng/ml, 15-23 ng/ml, 17-23 ng/ml, 19-23 ng/ml, 21-23 ng/ml, 5-21 ng/ml, 7-19 ng/ml, 5-13 ng/ml, 7-13 ng/ml, 9-13 ng/ml. In some embodiments, a presence or absence of hyper-acetylated HMGB1 can be determined. Accordingly, the predetermined level can optionally be 0. In some embodiments, the predetermined level can be a level that is at least 10% above the level of a control subject, for example, a healthy individual, or an asbestos-exposed individual. In some aspects the predetermined level can be a level that is at least 5% up to about 100% or up to about 200% or more, greater than the level of the comparison subject, or any value or subrange therebetween.

Optionally, the subject is identified as having MM when the level of disulfide HMGB1 (or level of antibody bound to disulfide HMGB1) in the sample is greater than a level of disulfide HMGB1 (or level of antibody bound to disulfide HMGB1) of about 0.1 ng/ml of disulfide HMGB1, for example greater than about 0.1, 0.2, 0.3, 0.4, 0.5, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 13, 14, 15, 16, 17, 18, 19, 20, 21, 22, 23, 24, 25, 30, 35, 40, 45, 50, 60, 70, 80, 90, 100, 150, or 200 ng/mL disulfide HMGB1, including ranges between any two of the listed values. Optionally, a level of antibody bound to about 5 ng/ml of disulfide HMGB1 indicates a presence of MM. Optionally, a level of antibody bound to about 3-23 ng/ml of disulfide HMGB1 indicates a presence of MM, for example about, 3-21 ng/ml, 3-19 ng/ml, 3-17 ng/ml, 3-15 ng/ml, 3-13 ng/ml, 3-11 ng/ml, 3-9 ng/ml, 3-7 ng/ml, 3-5 ng/ml, 5-23 ng/ml, 7-23 ng/ml, 9-23 ng/ml, 11-23 ng/ml, 13-23 ng/ml, 15-23 ng/ml, 17-23 ng/ml, 19-23 ng/ml, 21-23 ng/ml, 5-21 ng/ml, 7-19 ng/ml, 5-13 ng/ml, 7-13 ng/ml, 9-13 ng/ml disulfide HMGB1. Optionally, the subject is diagnosed as having MM when the level of antibody bound to disulfide HMGB1 in the sample is greater than two-fold above the lower limit of detection for the antibody, for example greater than two-, three-, four-, five-, six-, seven-, eight-, nine-, ten-, twenty-, thirty-, forty-, or fifty-fold.

Optionally, the subject is identified as having MM when the level of fully reduced HMGB1 (or antibody bound to fully reduced HMGB1) in the sample is greater than a level of fully reduced HMGB1 (or the antibody bound to fully reduced HMGB1) of about 3 ng/ml of fully reduced HMGB1, for example greater than about 3, 3.5, 4, 4.5, 5, 5.5, 6, 6.5, 7, 7.5, 8, 8.5, 9, 9.5, 10, 10.5, 11, 11.5, 12, 13, 14, 15, 16, 17, 18, 19, 20, 21, 22, 23, 24, 25, 30, 35, 40, 45, 50, 60, 70, 80, 90, 100, 150, or 200 ng/mL fully reduced HMGB1, including ranges between any two of the listed values. It is noted that subjects that were exposed to asbestos have been observed to have higher levels of fully reduced HMGB1 than healthy controls, and that subjects with MM were observed to have even higher levels of HMGB1 (see FIG. 1F). Optionally, a level of antibody bound to about 3-23 ng/ml of fully reduced HMGB1 indicates a presence of MM, for example about, 3-21 ng/ml, 3-19 ng/ml, 3-17 ng/ml, 3-15 ng/ml, 3-13 ng/ml, 3-11 ng/ml, 3-9 ng/ml, 3-7 ng/ml, 3-5 ng/ml, 5-23 ng/ml, 7-23 ng/ml, 9-23 ng/ml, 11-23 ng/ml, 13-23 ng/ml, 15-23 ng/ml, 17-23 ng/ml, 19-23 ng/ml, 21-23 ng/ml, 5-21 ng/ml, 7-19 ng/ml, 5-13 ng/ml, 7-13 ng/ml, 9-13 ng/ml. Optionally, the subject is diagnosed as having MM when the level of fully reduced antibody bound to disulfide HMGB1 in the sample is greater than two-fold above the lower limit of detection for the antibody, for example greater than two-, three-, four-, five-, six-, seven-, eight-, nine-, ten-, or twenty-, thirty-, forty-, or fifty-fold.

It has been observed that both asbestos-exposed individuals and MM patients have higher levels of hypo-acetylated HMGB1 than healthy controls (see FIG. 1D). As such, a presence or level of hypo acetylated HMGB1 can be useful, for example, for detecting false positives by healthy subjects. Optionally, the method further comprises detecting a level of antibody bound to hypo-acetylated HMGB1, and comparing the level of antibody bound to hypo-acetylated HMGB1 to a predetermined level of antibody bound to hypo-acetylated HMGB1. The predetermined level can be least about 0 ng/ml hypo-acetylated HMGB1, for example, 0, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 ng/ml, including ranges between any two of the listed values. Optionally, the predetermined level of hypo-acetylated HMGB1 (or level of antibody bound to hypo-acetylated HMGB1) comprises a range of hypo-acetylated HMGB1 values, for example, about 0-12 ng/ml, 0-10 ng/ml, 0-8 ng/ml, 0-6 ng/ml, 0-4 ng/mL, 0-2 ng/ml. In some embodiments, the predetermined level is at least 5% above the level of a comparison subject, for example, a subject that has no disease or exposure or a subject that has a different disease (e.g., lung cancer v. mesothelioma). In some aspects the predetermined level can be a level that is at least 5% up to about 100% or up to about 200% or more, greater than the level of the comparison subject, or a range between any two of these values.

It has been observed that both asbestos-exposed individuals and MM patients have higher levels of total HMGB1 than healthy controls (see Tables 3.1-3.2). Accordingly, in some embodiments, the method further comprises contacting the sample with an antibody that binds specifically to HMGB1, but detects total HMGB1 (e.g. an antibody that binds to an epitope common to all of the HMGB1 isoforms, or a polyclonal antibody against HMGB1). Optionally, the method comprises comparing the level of total HMGB1 to a predetermined level. The predetermined level can comprise at least 2 ng/mL of total HMGB1, for example at least about 2 ng/mL, 2.5, 3, 4, 5, 6, 7, 8, 9, 10, 11, 12, 13, 14, 15, 20, 25, 30, 35, 40, 45, 50, 60, 70, 80, 90, 100, 150, or 200 ng/mL, including ranges between any two of the listed values. It is noted that the predetermined level of total HMGB1 may be higher if the subject is a smoker rather than a non-smoker (see Tables 3.1-3.2). For example, in some embodiments, if the subject is a smoker, the predetermined level can be increased (compared to a non-smoker subject) by at least 5 ng/mL, for example, 5 ng/mL, 6, 7, 8, 9, 10, 11, 12, 13, 14, 15, 16, 17, 18, 19, or 20 ng/mL. In some embodiments, the predetermined level of total HMGB1 is 11.35 ng, and can differentiate subjects with MM (greater than 11.35 ng/ml) from subject with benign or malignant non-MM pleural effusions (less than 11.35 ng/ml). In some embodiments, the predetermined level of total HMGB1 is 15.75 ng/ml, and can differentiate subjects with MM (greater than 15.75 ng/ml) from subjects with benign or malignant non-MM pleural effusions (less than 15.75 ng/ml).

In some embodiments, if the HMGB1 or isoform(s) of HMGB1 are above the predetermined level, the method further comprises recommending or prescribing a treatment regimen for malignant mesothelioma or inflammatory cancer. Examples of treatment regimens include chemotherapy and radiation therapy.

Methods of Differentiating Malignant Mesothelioma from Asbestos Exposure

It has been observed herein that the presence and or levels of HMGB1 isoforms can differ between asbestos-exposed individuals and MM patients. It has been observed by mass spectrometry that the total amount of HMGB1 is significantly higher in MM patients than in asbestos-exposed individuals (see FIG. 1A). It has been observed by mass spectrometry that the ratio of hyper-acetylated HMGB1 to hypo-acetylated HMGB1 in MM patients is significantly higher than that in asbestos-exposed individuals (see FIG. 1D and FIG. 1E). It has been observed by mass spectrometry that the ratio of disulfide HMGB1 to fully reduced HMGB1 is significantly higher than that in asbestos-exposed individuals (see FIG. 1F). Accordingly, in some embodiments, methods for distinguishing, differentiating, identifying, or diagnosing subjects having MM from subjects exposed to asbestos are provided.

In some embodiments, a method of differentiating between malignant mesothelioma and asbestos-exposure in a subject is provided. The method can comprise providing a biological sample from a subject suspected of having malignant mesothelioma. The methods can comprise contacting the biological sample with at least one antibody that binds to an HMGB1 isoform of interest, for example, an antibody that binds specifically to hyper-acetylated HMGB1, an antibody that binds specifically to disulfide HMGB1 (HMGB1C23-C45), or an antibody that binds specifically to fully reduced HMGB1. The method can comprise detecting a level of the antibody bound to hyper-acetylated HMGB1, disulfide HMGB1 (HMGB1C23-C45) or chemokine HMGB1. The method can comprise comparing the level of antibody bound to hyper-acetylated HMGB1, disulfide HMGB1, or chemokine HMGB1 to a predetermined level, in which a level of hyper-acetylated HMGB1, disulfide HMGB1, or chemokine HMGB1 greater than a predetermined level indicates a presence of malignant mesothelioma in the subject.

Optionally, the subject is determined to have MM instead of being exposed to asbestos, when the level of hyper-acetylated HMGB1 (or level of antibody bound to hyper-acetylated HMGB1) from the sample is greater than a predetermined level, below which is characteristic of a healthy population and/or asbestos-exposed individuals. Example predetermined levels include levels of hyper-acetylated HMGB1 (or antibody bound to hype-acetylated HMGB1) of at least 0.1 ng/ml of hyper-acetylated HMGB1, for example 0.1 ng/ml, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, or 200 ng/ml hyper-acetylated HMGB1, including ranges between any two of the listed values. Optionally, a level of antibody bound to about 0.1-2 ng/ml, 0.1-1.8 ng/ml, 0.1-1.6 ng/ml, 0.1-1.4 ng/ml, 0.1-1.2 ng/mL, 0.1-1 ng/ml, 0.1-0.8 ng/ml, 0.1-0.6 ng/ml, 0.1-0.4 ng/ml, 0.1-0.2 ng/mL, 0.2-0.3 ng/ml, 0.2-0.5 ng/ml, 0.3-0.5 ng/ml, 0.3-0.7 ng/mL, 0.3-1 ng/ml, 0.5-1.2 ng/ml hyper-acetylated HMGB1 indicates that the subject has MM instead of asbestos. The term “about” as used herein when referring to a measurable value such as an amount, is meant to encompass variations of +/−20%, +/−10%, +/−5%, +/−1%, +/−0.5%, or +/−0.1%, or any number in between these percentages, from the specified value. In some aspects the level of antibody bound to hypo-acetylated HMGB1 includes ranges between any of the two listed values.

Optionally, a subject is identified as having MM when the level of disulfide HMGB1 (or antibody bound to disulfide HMGB1) is above a predetermined level. Example predetermined levels include levels disulfide HMGB1 (or antibody bound to disulfide HMGB1) of at least 0.1 ng/ml of disulfide HMGB1, for example 0.1 ng/ml, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, or 200 ng/ml disulfide HMGB1, including ranges between any two of the listed values. Optionally, a level of antibody bound to about 0.1-2 ng/ml, 0.1-1.8 ng/ml, 0.1-1.6 ng/ml, 0.1-1.4 ng/ml, 0.1-1.2 ng/mL, 0.1-1 ng/ml, 0.1-0.8 ng/ml, 0.1-0.6 ng/ml, 0.1-0.4 ng/ml, 0.1-0.2 ng/mL, 0.2-0.3 ng/ml, 0.2-0.5 ng/ml, 0.3-0.5 ng/ml, 0.3-0.7 ng/mL, 0.3-1 ng/ml, 0.5-1.2 ng/ml disulfide HMGB1 indicates that the subject has MM instead of asbestos. Optionally, the predetermined level of disulfide HMGB1 (or antibody bound to HMGB1) represents a fold increase (FI) over the lower limit of detection (LLD) for the antibody. In some embodiments, the predetermined level is a FI of at least about 0.5 FI over LLD, for example, about 0.5 FI over LLD, 0.7, 1, 1.5, 2, 2.5, 3, 3.5, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 400, 500, 600, 700, 800, 900, 1000, 2000, 5000, or 10,000 FI over LLD, including ranges between any two of the listed values. In some embodiments, an individual is identified as having MM when the level of antibody bound to disulfide HMGB1 is within about 3-41 Fl over LLD, 3-39 Fl over LLD, 3-37 Fl over LLD, 3-35 Fl over LLD, 3-33 Fl over LLD, 3-31 Fl over LLD, 3-29 Fl over LLD, 3-27 Fl over LLD, 3-25 Fl over LLD, 3-23 Fl over LLD, 5-21 Fl over LLD, 5-19 Fl over LLD, 7-17 Fl over LLD, 9-15 Fl over LLD, 11-13 Fl over LLD, 7-31 Fl over LLD, 9-29 Fl over LLD, 11-27 Fl over LLD, 7-33 Fl over LLD, 9-27 Fl over LLD, 9-25 Fl over LLD.

Optionally, the subject is determined to have asbestos exposure, when the level of fully reduced HMGB1 in the sample is greater than a predetermined level, below which is characteristic of healthy individuals, but below a level characteristic of MM. Accordingly, a range of fully reduced HMGB1 can identify an individual with asbestos exposure, for example a level of, or a level of fully-reduced HMGB1 (or antibody bound to fully reduced HMGB1) about 1-11 Fl over LLD, 1-10 Fl over LLD, 1-9 Fl over LLD, 1-8 Fl over LLD, 1-7 Fl over LLD, 1-6 Fl over LLD, 1-5 Fl over LLD, 1-4 Fl over LLD, 1-3 Fl over LLD, 2-9 Fl over LLD, 2-8 Fl over LLD, 2-7 Fl over LLD, 2-6 Fl over LLD, 2-5 Fl over LLD, 3-8 Fl over LLD, or 3-7 Fl over LLD.

### Methods of Identifying Asbestos Exposure, Malignant Mesothelioma, or Inflammatory Cancer

In some embodiments, a method of identifying asbestos exposure, malignant mesothelioma, or inflammatory cancer in a subject is provided. The method can comprise determining a presence or level of an isoform of HMGB1 in a biological sample of the subject. The isoform of HMGB1 can be selected from a 24.585 KDa isoform of HMGB1, a 24.587 KDa isoform of HMGB1, a 25.467 KDa isoform of HMGB1; or a 25.469 KDa isoform of HMGB1. In some embodiments, the presence of one or more of the listed isoforms is determined. In some embodiments, the presence of two, three, or four of the listed isoforms is determined. In some embodiments, the level of one or more of the listed isoforms is determined. In some embodiments, the level of two, three, or four of the listed isoforms is determined. The method can comprise comparing an amount of the isoform of HMGB1 to a predetermined level, wherein an amount greater than the predetermined level identifies asbestos exposure, malignant mesothelioma, or inflammatory cancer in the subject. Optionally, the method can comprise comparing an amount of the isoform of HMGB1 to an amount of the same isoform in a negative control sample from an individual known not to have mesothelioma and/or inflammatory cancer.

Optionally, the isoform or isoforms of HMGB1 are detected by mass spectrometry. Optionally, the isoform or isoforms of HMGB1 are detected by contacting the biological sample with an antibody that binds specifically to any of the isoforms, as described herein. Optionally, the isoform or isoforms of HMGB1 are detected by contacting the biological sample with an antibody that binds specifically to any of the isoforms as described herein and by performing mass spectrometry.

Optionally, mass spectrometry is performed to detect isoforms of HMGB1. Mass spectrometry is a technique that helps measure the amount and type of chemicals present in a sample. It analyzes the mass-to-charge ratio and presence of gas-phase ions. Optionally, Mass Spectrometry/Mass Spectrometry (MS/MS), in which a particular characteristic peak of a mass spec profile is further analyzed (e.g. sequenced). This is also known as Triple Quadruple Mass Spectrometry. In some aspects, Electrospray Ionization Liquid Chromatography Mass Spectrometry (ESI-LC-MS) can be used to observe the molecular ions as well as structural information of HMGB1 isoforms. And optionally, Matrix-assisted Laser desorption/ionization (MALDI) can be used to detect HMGB1 isoforms.

In some embodiments, an amount of an isoform of HMGB1 in a biological sample of the subject, including a) a 24.585 KDa isoform of HMGB1, b) a 24.587 KDa isoform of HMGB1, c) a 25.467 KDa isoform of HMGB1 or d) a 25.469 KDa isoform of HMGB1, is compared to an amount of the isoform of HMGB1 to a predetermined level, wherein an amount greater than the predetermined level identifies asbestos exposure, malignant mesothelioma, or inflammatory cancer in the subject. For example, a level of (c) 25.467 KDa isoform of HMGB1 or (d) 25.469 KDa isoform of HMGB1 greater than 5 ng/mL can indicate a presence of mesothelioma, and wherein a level of (a) 24.585 KDa isoform of HMGB1 or (d) 25.469 KDa isoform of HMGB1 greater than 5-fold of a lower limit of detection indicates a presence of mesothelioma or inflammatory cancer.

Optionally, the subject is identified as having an inflammatory cancer when the amount of HMGB1 isoform is above a predetermined level. Example inflammatory cancers include, but are not limited to, mesothelioma, colorectal cancer, pancreatic cancer, breast cancer, liver cancer, and the like.

### Kits

Some embodiments relate to immunoassay kits. The kits can comprise one or more antibodies that bind specifically to an isoform of HMGB1. The kit can further include a detectable moiety. By way of example, such kits can be useful for diagnosing or detecting MM and exposure to asbestos. The kits can be for use in any of a variety of immunoassays, for example, ELISA, Western Blot, later flow assays, no-wash assays, and the like, and can include reagents for any of these assays.

The kits can comprise at least one of the following antibodies, for example, one, two, three, four, five, six, seven, or eight of the antibodies: (a) an antibody that binds specifically to hyper-acetylated HMGB1; (b) an antibody that binds specifically to disulfide HMGB1 (HMGB1C23-C45); (c) an antibody that binds specifically to hypo-acetylated HMGB1; (d) an antibody that binds specifically to fully reduced HMGB1; (e) an antibody that binds specifically to hyper-acetylated disulfide HMGB1; (f) an antibody that binds specifically to hyper-acetylated fully reduced HMGB1; (g) an antibody that binds specifically to hypo-acetylated disulfide HMGB1; or (h) an antibody that binds specifically to hypo-acetylated fully-reduced HMGB1.

Optionally, the kit further comprises an antibody that binds to HMGB1, but does not necessarily bind to a particular isoform, for example, polyclonal antibody against HMGB1.

The kits can comprise one or more detectable moieties. Example detectable moieties include fluorophores, radiolabels, FRET pairs, enzyme-substrate pairs, enzyme, and the like as described herein. Optionally, any of the antibodies of (a), (b), (c), (d), (e), (f), (g), or (h) comprises a detectable moiety. Optionally, the kit comprises a secondary antibody that binds specifically to any of the antibodies of (a), (b), (c), (d), (e), (f), (g), or (h), and the secondary antibody comprises a detectable moiety. In some embodiments, for example in embodiments that contain two of more of the antibodies of (a), (b), (c), (d), (e), (f), (g), or (h), a first of the antibodies comprises a first detectable moiety, and a second of the antibodies comprises a second detectable moiety. Optionally, the second detectable moiety is different from the first detectable moiety. In some embodiments, each primary antibody of the kit (for example, antibodies of (a), (b), (c), (d), (e), (f), (g), or (h)) comprises a different detectable moiety. In some embodiments, the kit comprises secondary antibodies, and each secondary antibody of the kit comprises a different detectable moiety.

Optionally, the kit comprises a combination or panel of two antibodies that bind to different HMGB1 isoforms. Example combinations include (a) an antibody that binds specifically to hyper-acetylated HMGB1 and (b) an antibody that binds specifically to disulfide HMGB1 (HMGB1C23-C45); (a) an antibody that binds specifically to hyper-acetylated HMGB1 and (c) an antibody that binds specifically to hypo-acetylated HMGB1; (a) an antibody that binds specifically to hyper-acetylated HMGB1 and (d) an antibody that binds specifically to fully reduced HMGB1; (a) an antibody that binds specifically to hyper-acetylated HMGB1 and (e) an antibody that binds specifically to hyper-acetylated disulfide HMGB1; (a) an antibody that binds specifically to hyper-acetylated HMGB1 and (f) an antibody that binds specifically to hyper-acetylated fully reduced HMGB1; (a) an antibody that binds specifically to hyper-acetylated HMGB1 and (g) an antibody that binds specifically to hypo-acetylated disulfide HMGB1; (a) an antibody that binds specifically to hyper-acetylated HMGB1 and (h) an antibody that binds specifically to hypo-acetylated fully-reduced HMGB1; (b) an antibody that binds specifically to disulfide HMGB1 (HMGB1C23-C45) and (c) an antibody that binds specifically to hypo-acetylated HMGB1; (b) an antibody that binds specifically to disulfide HMGB1 (HMGB1C23-C45) and (d) an antibody that binds specifically to fully reduced HMGB1; (b) an antibody that binds specifically to disulfide HMGB1 (HMGB1C23-C45) and (e) an antibody that binds specifically to hyper-acetylated disulfide HMGB1; (b) an antibody that binds specifically to disulfide HMGB1 (HMGB1C23-C45) and (f) an antibody that binds specifically to hyper-acetylated fully reduced HMGB1; (b) an antibody that binds specifically to disulfide HMGB1 (HMGB1C23-C45) and (g) an antibody that binds specifically to hypo-acetylated disulfide HMGB1; (b) an antibody that binds specifically to disulfide HMGB1 (HMGB1C23-C45) and (h) an antibody that binds specifically to hypo-acetylated fully-reduced HMGB1; (c) an antibody that binds specifically to hypo-acetylated HMGB1 and (d) an antibody that binds specifically to fully reduced HMGB1; (c) an antibody that binds specifically to hypo-acetylated HMGB1 and (e) an antibody that binds specifically to hyper-acetylated disulfide HMGB1; (c) an antibody that binds specifically to hypo-acetylated HMGB1 and (f) an antibody that binds specifically to hyper-acetylated fully reduced HMGB1; (c) an antibody that binds specifically to hypo-acetylated HMGB1 and (g) an antibody that binds specifically to hypo-acetylated disulfide HMGB1; (c) an antibody that binds specifically to hypo-acetylated HMGB1 and (h) an antibody that binds specifically to hypo-acetylated fully-reduced HMGB1; (d) an antibody that binds specifically to fully reduced HMGB1 and (e) an antibody that binds specifically to hyper-acetylated disulfide HMGB1; (d) an antibody that binds specifically to fully reduced HMGB1 and (f) an antibody that binds specifically to hyper-acetylated fully reduced HMGB1; (d) an antibody that binds specifically to fully reduced HMGB1 and (g) an antibody that binds specifically to hypo-acetylated disulfide HMGB1; (d) an antibody that binds specifically to fully reduced HMGB1 and (h) an antibody that binds specifically to hypo-acetylated fully-reduced HMGB1; (e) an antibody that binds specifically to hyper-acetylated disulfide HMGB1 and (f) an antibody that binds specifically to hyper-acetylated fully reduced HMGB1; (e) an antibody that binds specifically to hyper-acetylated disulfide HMGB1 and (g) an antibody that binds specifically to hypo-acetylated disulfide HMGB1; (e) an antibody that binds specifically to hyper-acetylated disulfide HMGB1 and (h) an antibody that binds specifically to hypo-acetylated fully-reduced HMGB1; (f) an antibody that binds specifically to hyper-acetylated fully reduced HMGB1 and (g) an antibody that binds specifically to hypo-acetylated disulfide HMGB1; (f) an antibody that binds specifically to hyper-acetylated fully reduced HMGB1 and (h) an antibody that binds specifically to hypo-acetylated fully-reduced HMGB1; or (g) an antibody that binds specifically to hypo-acetylated disulfide HMGB1.

Optionally, the kit comprises a combination or panel of three antibodies. Example combinations include antibodies that bind to (a) hyper-acetylated HMGB1, (b) disulfide HMGB1 (HMGB1C23-C45), and (c) hypo-acetylated HMGB1; antibodies that bind to (a) hyper-acetylated HMGB1, (b) disulfide HMGB1 (HMGB1C23-C45), and (e) hyper-acetylated disulfide HMGB1; antibodies that bind to (a) hyper-acetylated HMGB1, (f) hyper-acetylated fully reduced HMGB1, (h) hypo-acetylated fully-reduced HMGB1; antibodies that bind to (a) hyper-acetylated HMGB1 and (g) hypo-acetylated disulfide HMGB1, and (h) hypo-acetylated fully-reduced HMGB1; antibodies that bind to (a) hyper-acetylated HMGB1, (e) hyper-acetylated disulfide HMGB1, and (g) hypo-acetylated disulfide HMGB1.

Optionally, the kit comprises a combination or panel of four antibodies. Example combinations include antibodies that bind to (a) hyper-acetylated HMGB1, (b) disulfide HMGB1 (HMGB1C23-C45), (c) hypo-acetylated HMGB1, and (d) fully reduced HMGB1; antibodies that bind to (a) hyper-acetylated HMGB1, (b) disulfide HMGB1 (HMGB1C23-C45), (d) fully reduced HMGB1, (g) hypo-acetylated disulfide HMGB1; antibodies that bind to (a) hyper-acetylated HMGB1, (b) disulfide HMGB1 (HMGB1C23-C45), (e) hyper-acetylated disulfide HMGB1, and (g) hypo-acetylated disulfide HMGB1.

In some embodiments, kit comprises a mass spectrometry kit. The kit can comprise a standard known to comprise or consist essentially of an isoform of HMGB1, for example (a) a 24.585 KDa isoform of HMGB1, (b) a 24.587 KDa isoform of HMGB1, (c) a 25.467 KDa isoform of HMGB1, (d) a 25.469 KDa isoform of HMGB1. In some embodiments, the standard comprises or consists essentially of two, three, or four HMGB1 isoforms, for example, (a) and (b), (a) and (c), (a) and (d), (b) and (c), (b) and (d), (c) and (d), (a) and (b) and (c), (a) and (b) and (d), (a) and (c) and (d), (b) and (c) and (d), or (a) and (b) and (c) and (d). In some embodiments, the standard comprises a known level or quantity of the indicated HMGB1 isoform(s). Optionally, the kit can comprise an antibody that binds specifically to HMGB1.

A subject kit may further include positive and negative controls. An example of a positive control is a sample, for example a biological sample, known to comprise the HMGB1 isoform (or isoforms) of interest. An example of a negative control is a sample for example a biological sample, which does not comprise the HMGB1 isoform (or isoforms) of interest. In some embodiments, the kits are useful in diagnostic applications, as described in detail herein. In some embodiments, the kit comprises a positive control, for example a substance comprising, or consisting essentially of only a single isoform of HMGB1, for example a 24.585 KDa isoform of HMGB1, a 24.587 KDa isoform of HMGB1, a 25.467 KDa isoform of HMGB1, or a 25.469 KDa isoform of HMGB1, or any two, three, or four of the above-referenced isoforms. The positive control can comprise known concentration or quantity of the indicated HMGB1 isoform(s). In some embodiments, the kit comprises a negative control, for example a substance known not to comprise one or more HMGB1 isoforms. Optionally, the negative control does not comprise any HMGB1.

A subject kit can further include, if desired, one or more of various conventional components, such as, for example, containers with one or more buffers (e.g., wash buffers), detection reagents or antibodies. Printed instructions, either as inserts or as labels, indicating quantities of the components to be used and guidelines for their use, can also be included in the kit. In the present disclosure it should be understood that the specified materials and conditions can be useful in accordance with some embodiments herein, but that unspecified materials and conditions are not excluded so long as they do not prevent the benefits of the invention from being realized.

A kit will in some embodiments provide a standard for normalization of a level of a target polypeptide to a standard, e.g., a level of an actin polypeptide, a level of a GAPDH polypeptide, etc. A kit will in some embodiments further include negative controls, e.g., antibodies specific for a non-target polypeptide; and the like.

Optionally, the kits can also include reagents for carrying out ELISAs (e.g., multi-well plates, 96-well plates; plates containing wells in multiples of 96, and the like). Optionally, the kits can also include substrates for lateral flow assays. Optionally, the kits can also include reagents for western blot. Optionally, the kits can also include components for conducting immunohistochemical analysis of a tissue sample (e.g. fixative, slides); and the like.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. flash drive, DVD-ROM, CD-ROM, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Additional Embodiments

Provided herein are embodiments based on the surprising and unexpected discovery that HMGB1 is highly expressed in malignant mesothelioma (MM) serum samples of MM patients and subjects exposed to asbestos. Also provided herein are embodiments drawn to the discovery that different HMGB1 isoforms are present in different stages of asbestos-induced MM pathogenesis. HMGB1 can be heavily regulated via post-translational modifications, in particular acetylation of lysines residues in its two nuclear localization signals (NLS1 or NLS2) and redox-sensitive modifications of its three cysteines. Upon exposure to asbestos fibers, HM passively released hypo-acetylated HMGB1 mostly in the fully reduced chemokine form. Supernatant from MM cells grown under hypoxic conditions to better mimic the tumor microenvironment presented conspicuous amount of actively secreted hyper-acetylated HMGB1, with the prevalent redox isoform being disulfide HMGB1. The long latency period between exposure to carcinogenic fibers and MM development provides physicians with a window for early diagnosis. As such, additional serological markers to differentiate asbestos-exposure and MM can be useful, since proposed biomarkers for MM—soluble mesothelin-related peptides (SMRPs), osteopontin, and fibulin-3—lack in sensitivity, specificity, or reproducibility.

Hyper-acetylated and disulfide HMGB1 isoforms have only been detected in non-malignant conditions like alcoholic liver disease (ALD), acute acetaminophen-induced liver failure, and severe macrophage activation syndrome. In larger cohorts of asbestos-exposed individuals, ALD might represent a relevant confounding factor. However, ALD patients present in their sera also a phosphorylated version of HMGB1, which is not present in healthy individuals, individuals exposed to asbestos, or MM patients. The levels of this phosphorylated isoform, together with accurate clinical history, physical examination, and other biochemical tests of liver function, can provide a further tool to distinguish ALD from MM in asbestos-exposed individuals with high HMGB1 levels. In some embodiments, a presence or level of phosphorylated HMGB1 isoforms above a predetermined level can identify a subject with Alcoholic Liver Disease (ALD).

### EXAMPLES

The following examples which are provided herein for purposes of illustration, and are not intended to be limiting.

For Examples 1-4, plasma samples from 19 MM patients, 20 asbestos-exposed individuals and 20 healthy controls were collected. HMGB1 isoforms were analyzed by ESI-LC-MS. Total and acetylated HMGB1 isoforms were expressed in ng/mL (median, 1st quartile-3rd quartile); redox HMGB1 isoforms were expressed as fold increase over lower limit of detection (FI over LLD) (median, 1st quartile-3rd quartile). Plasma osteopontin and fibulin-3 levels were measured in MM patients and asbestos-exposed individuals. The ability of these markers to distinguish cohorts was evaluated by the Mann-Whitney test for independent samples, without correction for multiple comparisons, and Receiver-Operating-Characteristic (ROC) curves. The area under the ROC curve (AUC) was calculated.

For Example 8, HMGB1 levels in supernatants of HM and MM cells in tissue culture were measured.

For Examples 9-11, serum samples from 20 unexposed healthy controls, 22 MM patients who had been diagnosed following the development of pleural effusion, 20 insulator workers that were exposed to asbestos due to their occupational history, and included individuals with 10 or more years of occupational asbestos exposure were collected and measured, 13 patients with cytologically benign pleural effusions, and 25 serum samples from patients with pleural effusions due to non-MM malignancy were measured. HMGB1 isoforms were analyzed by ESI-LC-MS. Total and acetylated HMGB1 isoforms were expressed in ng/mL (median, 1st quartile-3rd quartile). Plasma fibulin-3, mesothelin, and OPN levels were measured in MM patients, asbestos-exposed individuals, patients with benign pleural effusions, and patients with non-MM malignant pleural effusions. The ability of these markers to distinguish cohorts was evaluated by the Mann-Whitney test for independent samples, without correction for multiple comparisons, and Receiver-Operating-Characteristic (ROC) curves. The area under the ROC curve (AUC) was calculated.

### Example 1: Total and Isoform-Specific HMGB1 Levels

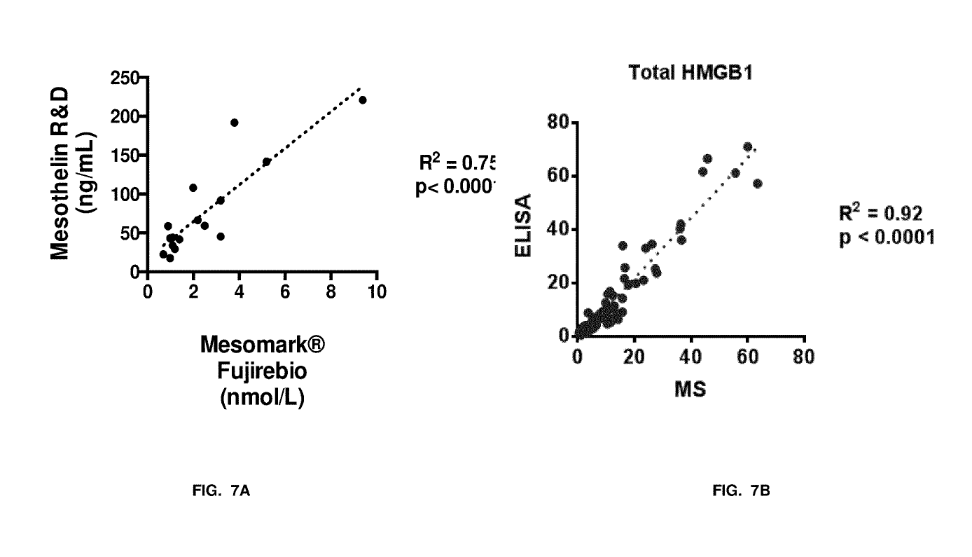

Total and isoform-specific HMGB1 in all three cohorts (healthy controls, MM patients, and asbestos-exposed individuals) were measured. Both a commercially available ELISA kit and a mass spectrometry protocol in accordance with methods and kits of some embodiments herein were used. The total levels of HMGB1 measured using these two different methods were very similar, (R²=0.92, P<0.0001) (FIG. 7B), corroborating the reliability of approaches described herein.

In the healthy control cohort, total HMGB1 was barely detectable (1.51±0.64 ng/mL). Levels significantly higher were measured in asbestos-exposed individuals (9.02±4.13 ng/mL, p<0.001) and MM patients, which showed the highest HMGB1 levels (30.51±17.30 ng/mL, p<0.001 when compared to either other group) (FIG. 1A).

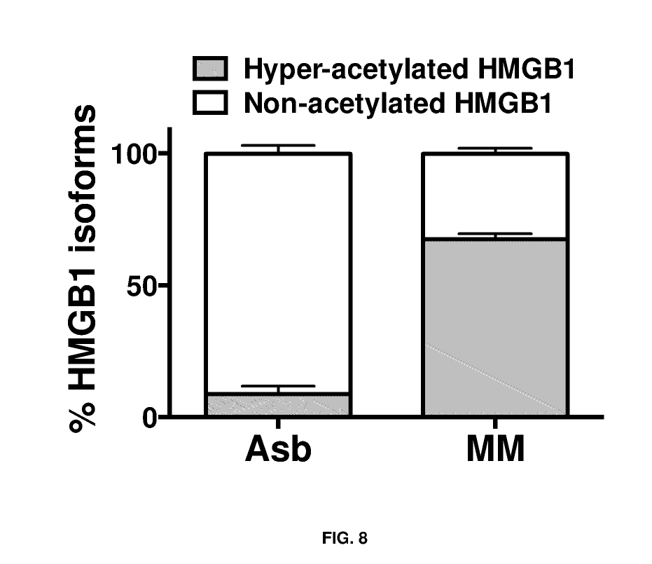

Total levels of HMGB1 for healthy individuals (H), asbestos-exposed individuals (Asb), and MM patients (MM) are also depicted in are also shown in FIG. 1G. Overall, hyper-acetylated HMGB1 comprised ˜10% of the total HMGB1 in the sera of asbestos-exposed individuals, and ˜67% of the total HMGB1 in the sera of MM patients (FIG. 8).

The HMGB1 isoform signature was highly consistent among all the individuals belonging to the same cohort. In asbestos-exposed individuals, fully reduced or chemokine HMGB1 was detected. This isoform was also characterized as being hypo-acetylated at NLS1 or NLS2 (FIG. 1B). In the MM cohort, four isoforms were detected: hypo-acetylated and hyper-acetylated fully reduced HMGB1, and hypo-acetylated and hyper-acetylated disulfide HMGB1 (FIG. 1C) Overall, hyper-acetylated HMGB1 comprised about 65% of total HMGB1 in MM patients, and only about 9% in asbestos-exposed individuals (FIG. 1E).

MM patients had also very high levels of hyper-acetylated HMGB1 (19.28±10.47 ng/mL), while healthy controls (0.47±0.23 ng/mL) and asbestos-exposed individuals (0.48±0.43 ng/mL) were virtually devoid of it (p<0.001 for both comparisons) (FIG. 1D). Compared to controls (1.04±0.61 ng/mL), the levels of hypo-acetylated HMGB1 were significantly higher in asbestos-exposed individuals (8.54±4.10 ng/mL, p<0.001) and MM patients (11.23±7.17 ng/mL, p<0.001), but the difference between asbestos-exposed individuals and MM patients was not significant (p=0.33) (FIG. 1H).

Disulfide cytokine HMGB1 was exclusively detected in sera from MM patients (19.12±11.42 FI over LLD) but could not be detected in sera from either asbestos-exposed or non-exposed healthy individuals since it is below the limit of detection (FIG. 1F). Instead, fully reduced chemokine HMGB1 were detected in all the three groups. Compared to the healthy control cohort (1.86±0.73 FI over LLD), fully reduced HMGB1 levels were significantly higher in asbestos-exposed individuals (5.73±2.69 FI over LLD, P<0.001), and even more so in MM patients (10.71±8.90 FI over LLD, P<0.001 vs. healthy controls, p<0.05 vs. asbestos-exposed individuals) (FIG. 1F). As such, a significant difference was found especially on disulfide HMGB1 among the MM patients, asbestos-exposed, and non-exposed healthy individuals.

Compared to controls (1.86±0.73 FI over LLD), fully reduced chemokine HMGB1 levels were significantly higher in asbestos-exposed individuals (5.73±2.69 FI over LLD, p<0.001), and even more so in MM patients (10.49±8.72 FI over LLD, p<0.001 vs. healthy controls, p<0.05 vs. asbestos-exposed individuals). Disulfide HMGB1 was exclusively detected in serum from MM patients (18.76±11.23 FI over LLD) (FIG. 1F). Thus a presence of disulfide HMGB1 (e.g. above the lower limit of detection) in accordance with methods and kits of some embodiments herein is indicate of MM, and can differentiate MM patients from healthy individuals and asbestos-exposed individuals.

Levels of disulfide HMGB1 and fully reduced HMGB1 for healthy individuals (H), asbestos-exposed individuals (Asb), and MM patients (MM) are also depicted in FIG. 1I.

The in vivo results described herein significantly overlapped with results previously observed in in vitro experiments. It has been observed that isoform-specific HMGB1 released from concentrated supernatants of primary mesothelial cells (HM) exposed to asbestos and MM cell lines. It has been observed herein that HM in normal tissue culture (unexposed controls) does not release detectable HMGB1 into extra-cellular space. Upon exposure to asbestos fibers, HM undergo programmed cell necrosis and passively released hypo-acetylated HMGB1 is observed (FIG. 1J). In the supernatant from MM cell cultures, only hyper-acetylated HMGB1 (actively secreted isoform) HMGB1 is observed (FIGS. 1K and 1L).

Accordingly, different HMGB1 isoforms can be used to differentiate between populations of subjects in accordance with some embodiments herein. In some embodiments, levels of hyper-acetylated HMGB1 above a predetermined level indicate that a subject has MM, while levels of hyper-acetylated HMGB1 below the predetermined level indicates that a subject does not have MM (but rather can be a healthy control or asbestos-exposed, non-MM subject).

### Example 2: Discrimination Between MM and Asbestos-Exposure

The performance of total and isoform-specific HMGB1 to discriminate asbestos-exposed individuals from MM patients was evaluated. Total and isoform-specific HMGB1 to proposed biomarkers such as mesothelin, osteopontin and fibulin-3 were compared.

The plasma levels of mesothelin were measured using a commercially available ELISA kit (R&D Systems, Minneapolis, Minn.) according to the manufacturer's instructions, and compared to the soluble mesothelin related peptides (0.4, 0.3-0.6 ng/ml) MESOMARK™ kit (Fujirebio Diagnostics Inc., Malvern, Pa.). These two testing methods show a very good correlation between the two kits (R2=0.75, P<0.0001) (FIG. 7A) when tested on a subset of samples from the cohort. Plasma levels of fibulin-3 and osteopoontin were measured in MM patients and asbestos-exposed individuals with commercially available ELISA kits following manufacturers' instructions (USCN, Wuhan, PRC for fibulin-3 and R&D Systems, Minneapolis, Minn. for osteopontin).

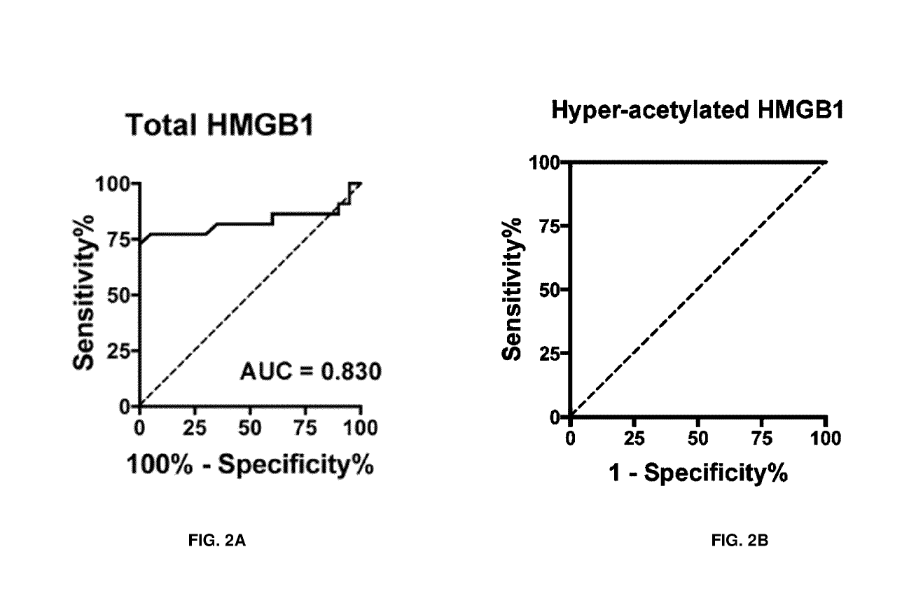

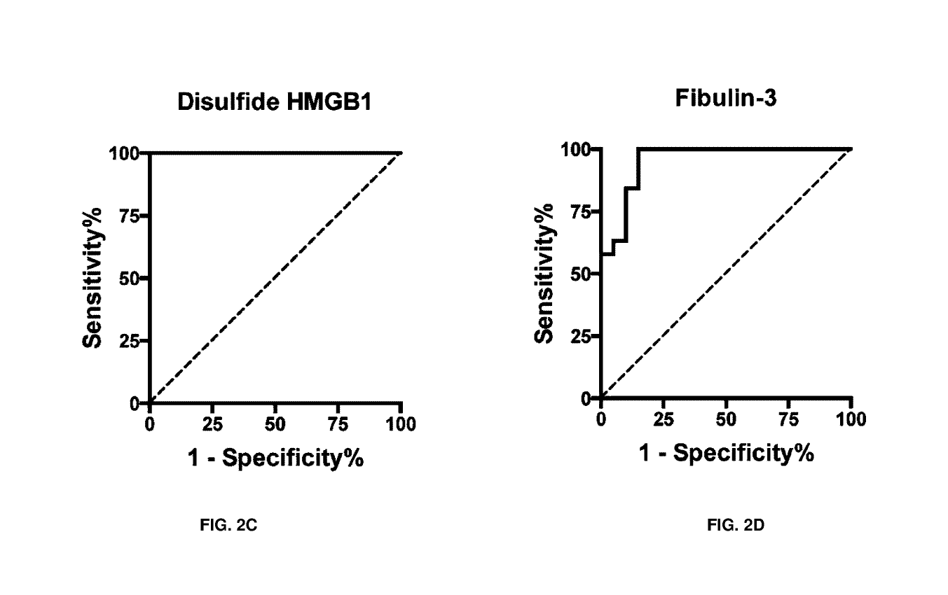

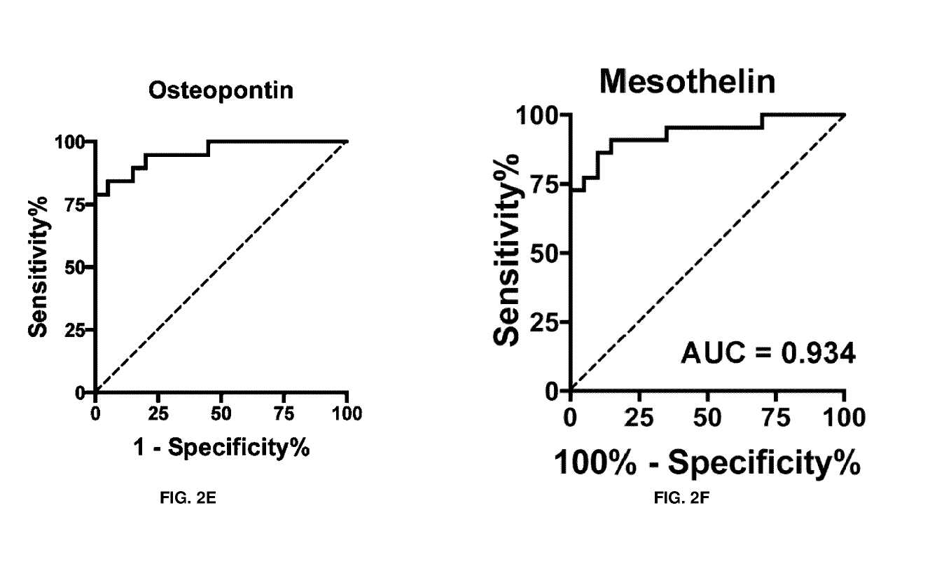

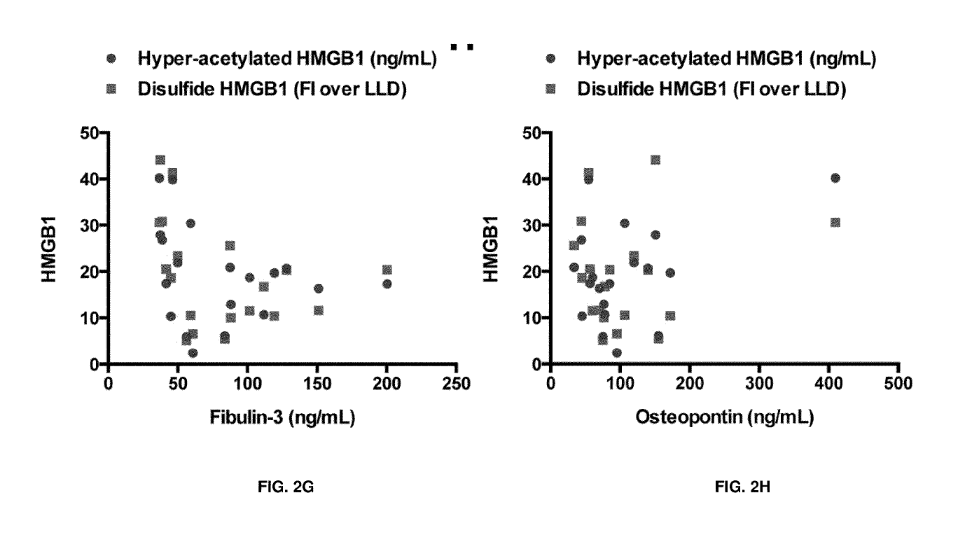

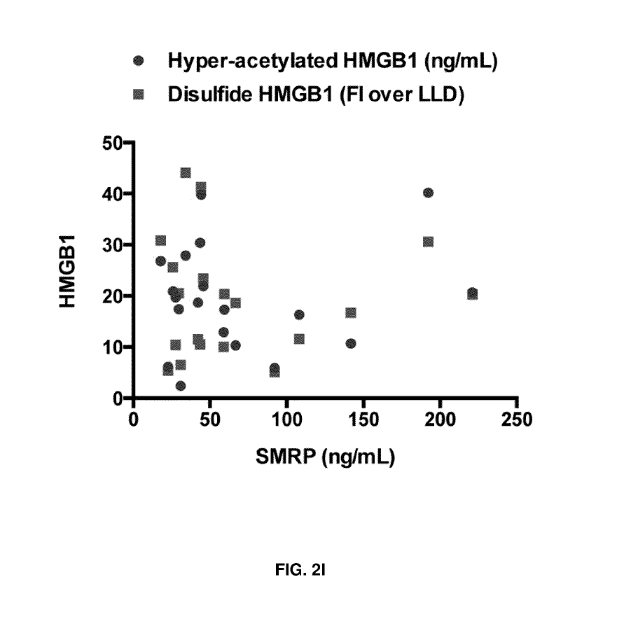

The AUC of total HMGB1 was 0.91 (0.80-1.00) (FIG. 2A); the sensitivity at a specificity of 100% was 85%; the specificity at a sensitivity of 100% was 10% (Table 2). The AUC (95% CI) for hyper-acetylated and disulfide HMGB1, the two isoforms typical of MM patients, were both 1.00 (1.00-1.00) [FIGS. 2B and 2C], with sensitivity and specificity of 100% for values respectively >2.15 ng/mL and >2.55 FI over LLD (Table 2).

TABLE 2

| | | | | Sensitivity at | Specificity at |
|---|---|---|---|---|---|
| | No. of | AUC | | 100% | 100% |
| Marker | Participants | (95% CI) | Cutoff | Specificity | Sensitivity |
| Total | 20 vs. 20 | 0.91 (0.80-1.00) | 15.75 ng/mL | 85% | 10% |
| HMGB1 | | | | | |
| Hyper- | | 1.00 (1.00-1.00) | 2.15 ng/mL | 100% | 100% |
| acetylated | | | | | |
| HMGB1 | | | | | |
| Disulfide | | 1.00 (1.00-1.00) | 2.55Fl | 100% | 100% |
| HMGB1 | | | over | | |
| | | | LLD | | |
| Osteopontin | | 0.95 (0.90-1.00) | — | — | — |
| Fibulin-3 | | 0.95 (0.89-1.00) | — | — | — |
| SMRP | | 0.96 (0.91-1.00) | — | — | — |

In this sample set, HMGB1 isoforms performed better in discriminating MM patients from asbestos-exposed individuals compared to osteopontin (see FIG. 2E), fibulin-3 (see FIG. 2D), and SMRP (see FIG. 2F).

In this sample set, the levels of isoform-specific HMGB1 in MM patients do not correlate with levels of osteopontin, fibulin-3, and SMRP. Without being limited by any theory, it is contemplated that these three molecules have independent roles in MM pathogenesis and progression of these molecules (see FIG. 2G, FIG. 2H, and FIG. 2I). It is further contemplated, however, that osteopontin, fibulin-3 and/or SMRP can provide additional information to HMGB1. As such, in some embodiments, methods further comprise detecting osteopontin, fibulin-3, SMRP, or a combination of these three in addition to the HMGB1 isoforms described herein. As such, in some embodiments, kits further comprise a reagent for detecting osteopontin, fibulin-3, SMRP, or a combination of these three in addition to the HMGB1 isoforms described herein.

Accordingly, detection of HMGB1 isoforms in accordance with some embodiments herein provides a greater sensitivity and greater specificity for differentiating between asbestos-exposed individuals and MM patients than total HMGB1 levels, or than levels of other markers such as osteopontin, fibulin-3, and/or SMRP.

### Example 3: Univariate and Multivariate Analyses of Total and Isoforms of HMGB1

Univariate and multivariate analyses were conducted to identify demographic variables that could influence levels of isoform-specific HMGB1 (see Tables 3.1-3.13).

For the statistical analysis, two age groups were defined: 55 years and less (N=30), and more than 55 years (n=29). Active and former smokers were grouped together and compared to non-smokers. When exposure to asbestos was known, exposed (long and short exposure) were grouped and compared to non-exposed. Using Barlett's test, it was found that HMGB1 and its isoforms presented significant heterogeneous variances between groups. Consequently, Mann and Whitney non-parametric test was used to compare two groups, and the Kruskall-Wallis non-parametric test was used to compare more than two groups. The significance was set to a p-value<0.05. The multivariate analysis of HMGB1 total and isoforms was carried out using a linear regression, after analyzing all interactions between the studied factors and the possible confounding due to each significant factor. Smoking status was found to be a confounding factor for total HMGB1; consequently the multivariate analysis of this variable was stratified on smoking status (see Tables 4.1-4.6). All statistical analyses were run using STATA.

Hyper-acetylated HMGB1 was strongly associated with MM, although its levels were slightly influenced by other factors (e.g. sex, age, smoking)(see Tables 3.3-3.4). Disulfide HMGB1 was strongly associated with MM, independently of demographic variables (see Tables 3.11-3.12).

In this sample set, the level of hyper-acetylated HMGB1 in 14 female subjects was 8.56±11.55 ng/mL, while the level of hyper-acetylated HMGB1 in 45 male subjects was 5.93±10.37 ng/mL (p<0.2529 for the comparison). The level of hyper-acetylated HMGB1 in 29 subjects older than 55 years old was 10.74±12.55 ng/mL, while the level of hyper-acetylated HMGB1 in 30 subjects younger than 55 years old was 2.46±6.19 ng/mL (p<0.0102 for the comparison). The level of hyper-acetylated HMGB1 in 25 subjects who are previous or currently active smokers was 10.48±11.99 ng/mL, while the level of hyper-acetylated HMGB1 in 33 non-smokers was 3.55±8.66 ng/mL (p<0.0436 for the comparison) (Tables 3.3-3.4).

In this sample set, the level of disulfide HMGB1 in 14 female subjects was 8.68±12.47 ng/mL, while the level of disulfide HMGB1 in 45 male subjects was 5.39±10.58 ng/mL (p<0.3730 for the comparison). The level of disulfide HMGB1 in 29 subjects older than 55 years old was 10.40±12.93 ng/mL, while the level of disulfide HMGB1 in 30 subjects younger than 55 years old was 2.09±6.87 ng/mL (p<0.0041 for the comparison). The level of disulfide HMGB1 in 25 subjects who are previous or currently active smokers was 9.75±12.33 ng/mL, while the level of disulfide HMGB1 in 33 non-smokers was 3.50±9.45 ng/mL (p<0.0445 for the comparison) (Table 3.11-3.12).

TABLE 3.1
Univariate analyses of total HMGB1

| Total HMGB1 | | | |
|---|---|---|---|
| Univariate analysis | N | Mean (ng/mL) [s.d.] | p value |
| Sex | | | 0.5040 |
| Male | 45 | 13.56 [15.66] | |
| Female | 14 | 12.87 [16.86] | |
| Age | | | 0.0003 |
| <=55 years | 30 | 6.84 [9.52] | |
| >55 years | 29 | 20.18 [18.16] | |
| Smoking status | | | 0.0001 |
| Non-smoker | 33 | 7.23 [12.89] | |
| Previous/active smoker | 25 | 21.76 [15.99] | |
| Asbestos exposure | | | 0.0001 |
| Never | 21 | 2.58 [4.93] | |
| <=20 yrs | 15 | 26.12 [19.44] | |
| 12 >20 yrs | 20 | 12.14 [8.91] | |
| unknown | 3 | 33.83 [22.70] | |
| MM | | | 0.0001 |
| No | 40 | 5.27 [4.79] | |
| Yes | 19 | 30.50 [17.30] | |
| MM vs asbestos exposed | | | 0.0001 |
| Non exposed vs asbestos exposed | | | 0.0001 |
| MM | 19 | 30.50 [17.30] | |
| Asb. exposed | 20 | 9.02 [4.12] | |
| Non exposed | 20 | 1.51 [0.64] | |

TABLE 3.2
Multivariate analyses of total HMGB1

| Total HMGB1 Multivariate analysis | Coef. | 95% CI | p value |
|---|---|---|---|
| In non-smokers- N = 33 | Linear regression R² = 0.5779 | | |
| MM | 19.40 | 9.67-29.12 | <0.001 |
| Asbestos | 6.67 | −1.31-14.64 | 0.098 |
| Sex | 3.91 | −3.42-11.24 | 0.284 |
| constant | −10.60 | −27.28-6.08 | 0.204 |
| In smokers - N = 25 | Linear regression R² = 0.6809 | | |
| MM | 28.03 | 19.54-36.51 | <0.001 |
| Sex | −16.32 | −29.36-−3.28 | 0.017 |
| constant | 26.58 | 12.46-40-71 | 0.001 |

TABLE 3.3
Univariate analyses of Hyper-acetylated HMGB1

| Hyper-acetylated HMGB1 | | | |
|---|---|---|---|
| Univariate analysis | N | Mean(ng/mL) [s.d.] | p value |
| Sex | | | 0.2529 |
| Male | 45 | 5.93 [10.37] | |
| Female | 14 | 8.56 [11.55] | |
| Age | | | 0.0102 |
| <=55 years | 30 | 2.46 [6.19] | |
| >55 years | 29 | 10.74 [12.55] | |
| Smoking status | | | 0.0436 |
| Non-smoker | 33 | 3.55 [8.66] | |
| Previous/active smoker | 25 | 10.48 [11.99] | |
| Asbestos exposure | | | 0.0237 |
| Never | 21 | 1.27 [3.68] | |
| <=20 yrs | 15 | 14.29 [13.21] | |
| >20 yrs | 20 | 3.76 [7.03] | |
| unknown | 3 | 23.00 [14.65] | |
| MM | | | 0.0001 |
| No | 40 | 0.475 [0.34] | |
| Yes | 19 | 19.28 [10.47] | |
| MM vs asbestos exposed | | | 0.0001 |
| Non exposed vs asbestos exposed | | | 0.4249 |
| MM | 19 | 19.28 [10.47] | |
| Asb. exposed | 20 | 0.48 [0.43] | |
| Non exposed | 20 | 0.47 [0.23] | |

TABLE 3.4
Multivariate analyses of Hyper-acetylated HMGB1

| Hyper-acetylated HMGB1 | | | |
|---|---|---|---|
| Multivariate analysis | | | |
| Patients with known smoking status - | Coef. | 95% CI | p value |
| N = 58 | Linear regression R² = 0.7290 | | |
| MM | 19.13 | MM | 19.13 |
| Smoking status | 1.23 | Smoking status | 1.23 |
| constant | 9.15 | 1.31-16.99 | 0.024 |

TABLE 3.5
Univariate analyses of Hypo-acetylated HMGB1

| Hypo-acetylated HMGB1 | | | |
|---|---|---|---|
| Univariate analysis | N | Mean (ng/mL) [s.d.] | p value |
| Sex | | | 0.0705 |
| Male | 45 | 7.63 [6.51] | |
| Female | 14 | 4.41 [5.36] | |
| Age | | | 0.0025 |
| <=55 years | 30 | 4.37 [4.83] | |
| >55 years | 29 | 9.44 [6.80] | |
| Smoking status | | | 0.0001 |
| Non-smoker | 33 | 3.67 [4.89] | |
| Previous/active smoker | 25 | 11.27 [5.54] | |
| Asbestos exposure | | | 0.0001 |
| Never | 21 | 1.30 [1.35] | |
| <=20 yrs | 15 | 11.83 [6.98] | |
| >20 yrs | 20 | 8.38 [4.52] | |
| unknown | 3 | 10.83 [8.06] | |
| MM | | | 0.0005 |
| No | 40 | 4.79 [4.78] | |
| Yes | 19 | 11.23 [7.17] | |
| MM vs asbestos exposed | | | 0.3252 |
| Non exposed vs asbestos exposed | | | 0.0001 |
| MM | 19 | 11.23 [7.17] | |
| Asb. exposed | 20 | 8.54 [4.10] | |
| Non exposed | 20 | 1.04 [0.61] | |

TABLE 3.6
Multivariate analyses of Hypo-acetylated HMGB1

| Hypo-acetylated HMGB1 | | | |
|---|---|---|---|
| Multivariate analysis | | | |
| Patients with known smoking | Coef. | 95% CI | p value |
| status - N = 58 | Linear regression R² = 0.5471 | | |
| Asbestos exposure | 5.22 | 1.85-8.58 | 0.003 |
| MM | 3.53 | 0.75-6.32 | 0.014 |
| Smoking status | 3.22 | 0.11-6.34 | 0.043 |
| constant | −7.30 | −11.69-−2.92 | 0.002 |

TABLE 3.7
Univariate analyses of Hypo-acetylated HMGB1

| Fully reduced HMGB1 | | | | |
|---|---|---|---|---|
| Univariate analysis | N | Mean (ng/mL) [s.d.] | | p value |
| Sex | | | | 0.2223 |
| Male | 45 | 6.48 | [6.79] | |
| Female | 14 | 4.56 | [4.57] | |
| Age | | | | 0.0089 |
| <=55 years | 30 | 4.10 | [4.00] | |
| >55 years | 29 | 8.02 | [7.67] | |
| Smoking status | | | | 0.0001 |
| Non-smoker | 33 | 3.71 | [4.41] | |
| Previous/active smoker | 25 | 9.27 | [7.22] | |
| Asbestos exposure | | | | 0.0001 |
| Never | 21 | 2.11 | [1.35] | |
| <=20 yrs | 15 | 10.61 | [9.72] | |
| >20 yrs | 20 | 6.09 | [3.27] | |
| unknown | 3 | 10.03 | [6.82] | |
| MM | | | | 0.0001 |
| No | 40 | 3.80 | [2.76] | |
| Yes | 19 | 10.71 | [8.90] | |
| MM vs asbestos exposed | | | | 0.0163 |
| Non exposed vs asbestos exposed | | | | 0.0001 |
| MM | 19 | 10.71 | [8.90] | |
| Asb. exposed | 20 | 5.73 | [2.69] | |
| Non exposed | 20 | 1.86 | [0.73] | |

TABLE 3.8
Multivariate analyses of Hypo-acetylated HMGB1

| Multivariate analysis | | | |
|---|---|---|---|
| Hypo-acetylated HMGB1 | | | |
| Patients with known smoking | Coef. | 95% CI | p value |
| status - N = 58 | Linear regression R² = 0.4046 | | |
| MM | 6.42 | 2.85-9.99 | 0.001 |
| Smoking status | 2.48 | −1.21-6.17 | 0.183 |
| Asbestos exposure | 2.14 | −2.27-6.56 | 0.334 |
| Sex | −1.61 | −5.20-1.97 | 0.371 |
| Age | −1.11 | −4.67-2.45 | 0.534 |
| constant | 0.71 | −7.78-9.20 | 0.868 |

TABLE 3.9
Univariate analyses of Percentage of Hyper-acetylated HMGB1

| Percentage of hyper-acetylated HMGB1 | | | | |
|---|---|---|---|---|
| Univariate analysis | N | Mean (%) [s.d.] | | p value |
| Sex | | | | 0.0108 |
| Male | 45 | 31.9% | [28.9%] | |
| Female | 14 | 50.2% | [24.1%] | |
| Age | | | | 0.2430 |
| <=55 years | 30 | 31.7% | [28.5%] | |
| >55 years | 29 | 28.7% | [28.7%] | |
| Smoking status | | | | 0.4653 |
| Non-smoker | 33 | 37.0% | [27.9%] | |
| Previous/active smoker | 25 | 33.6% | [29.7%] | |
| Asbestos exposure | | | | 0.4379 |
| Never | 21 | 38.7% | [26.7%] | |
| <=20 yrs | 15 | 42.2% | [27.3%] | |
| >20 yrs | 20 | 24.3% | [29.5%] | |
| unknown | 3 | 68.9% | [2.2%] | |
| MM | | | | 0.0001 |
| No | 40 | 22.8% | [25.2%] | |
| Yes | 19 | 64.5% | [6.2%] | |
| MM vs asbestos exposed | | | | 0.0013 |
| Non exposed vs asbestos exposed | | | | 0.0001 |
| MM | 19 | 64.5% | [6.2%] | |
| Asb. exposed | 20 | 8.6% | [14.0%] | |
| Non exposed | 20 | 37.0% | [26.2%] | |

TABLE 3.10
Multivariate analyses of Percentage of Hyper-acetylated HMGB1

| Percentage of Hyper-acetylated HMGB1 | | | |
|---|---|---|---|
| Multivariate analysis | | | |
| Patients with known smoking | Coef. | 95% CI | p value |
| status - N = 58 | Linear regression R² = 0.6198 | | |
| MM | 0.53 | 0.42-0.64 | <0.001 |
| Asbestos exposure | −0.27 | −0.37-−0.16 | <0.001 |
| constant | 0.63 | 0.46-0.80 | <0.001 |

TABLE 3.11
Univariate analyses of Disulfide HMGB1

| Disulfide HMGB1 | | | | |
|---|---|---|---|---|
| Univariate analysis | N | Mean (ng/mL) [s.d.] | | p value |
| Sex | | | | 0.3730 |
| Male | 45 | 5.39 | [10.58] | |
| Female | 14 | 8.68 | [12.47] | |
| Age | | | | 0.0041 |
| <=55 years | 30 | 2.09 | [6.87] | |
| >55 years | 29 | 10.40 | [12.93] | |
| Smoking status | | | | 0.0445 |
| Non-smoker | 33 | 3.50 | [9.45] | |
| Previous/active smoker | 25 | 9.75 | [12.33] | |
| Asbestos exposure | | | | 0.0036 |
| Never | 21 | 0.97 | [4.45] | |
| <=20 yrs | 15 | 14.72 | [13.94] | |
| >20 yrs | 20 | 2.95 | [6.44] | |
| unknown | 3 | 21.30 | [13.32] | |
| MM | | | | 0.0001 |
| No | 40 | 0.00 | | |
| Yes | 19 | 19.17 | [11.38] | |
| MM vs asbestos exposed | | | | 0.0001 |
| Non exposed vs asbestos exposed | | | | 1.000 |
| MM | 19 | 19.17 | [11.38] | |

| | | |
|---|---|---|
| Asb. exposed | 20 | 0.00 |
| Non exposed | 20 | 0.00 |

TABLE 3.12
Multivariate analyses of Disulfide HMGB1

| Multivariate analysis | | | |
|---|---|---|---|
| Patients with known smoking | Coef. | 95% CI | p value |
| status - N = 58 | Linear regression R² = 0.6944 | | |
| MM | 19.96 | 16.46-23.45 | <0.001 |
| constant | 0.00 | −1.95-1.95 | <0.001 |

s.d.: standard deviation - MM: malignant mesothelioma
All R² are very significant with a p-value <0.0001

TABLE 3.13
Multivariate analyses of all cohorts

| | | Total HMGB1 | Hyper-acetylated |
|---|---|---|---|
| Factor | N | (ng/mL) | HMGB1 (ng/mL) |
| Sex | | p = 0.4885 NS | p = 0.0621 NS |
| Male | 67 | 11.47 ± 13.36 | 5.97 ± 8.74 |
| Female | 33 | 9.49 ± 11.41 | 6.89 ± 7.77 |
| Age | | P = 0.0005*** | P = 0.0001*** |
| ≦55 years | 37 | 6.79 ± 8.73 | 3.00 ± 5.86 |
| >55 years | 63 | 13.18 ± 14.11 | 8.20 ± 9.09 |
| Smoking | | P = 0.0001*** | P = 0.0127* |
| No | 54 | 6.93 ± 10.31 | 4.24 ± 7.07 |
| Yes | 41 | 16.19 ± 14.46 | 8.87 ± 9.79 |
| Asbestos | | | |
| No vs Exposed | | P = 0.0001*** | P = 0.0001*** |
| Never | 22 | 3.51 ± 6.51 | 1.95 ± 4.81 |
| ≦20 yrs | 17 | 27.66 ± 20.08 | 15.71 ± 13.84 |
| >20 yrs | 20 | 12.14 ± 8.91 | 3.76 ± 7.03 |
| unknown | 41 | 7.11 ± 3.83 | 5.91 ± 3.35 |
| No vs short exp. | | p = 0.0001*** | p = 0.0024** |
| No vs long exp. | | p = 0.0001*** | p = 6413 NS |
| Short vs long exp. | | p = 0.0080** | P = 0.0055** |
| MM | | | |
| MM vs others | | p = 0.0001*** | p = 0.0001*** |
| No | 78 | 6.16 ± 4.33 | 3.11 ± 3.51 |
| Yes | 22 | 27.34 ± 18.12 | 17.49 ± 10.90 |
| Non-MM Exposed | 58 | | |
| MM vs other exposed | | p = 0.0001*** | p = 0.0001*** |
| Stage | | P = 0.3673 - NS | P = 0.3673 - NS |
| 1-2 | 9 | 29.2 ± 13.43 | 18.44 ± 7.93 |
| 3-4 | 13 | 26.05 ± 21.22 | 16.83 ± 12.83 |
| Effusion | | | |
| No vs Yes | | p = 0.0001*** | p = 0.0001*** |
| No | 40 | 5.27 ± 4.79 | 0.47 ± 0.34 |
| Yes | 60 | 15.04 ± 15.45 | 10.43 ± 9.30 |
| Benign | 13 | 6.82 ± 2.91 | 5.57 ± 2.48 |
| Malignant | 47 | 17.34 ± 16.73 | 11.79 ± 10.05 |
| Benign vs Malignant | | p = 0.0594 NS | p = 0.0762 NS |

MM: malignant mesothelioma - NS: non significant
*p < 0.05
**p < 0.01
***p < 0.001

Tables 4.1-4.6 provide multivariate models for total and isoforms of HMGB1.

TABLE 4.1
Multivariate models for total HMGB1

| Total HMGB1 | | | |
|---|---|---|---|
| Multivariate analysis | Coef. | 95% CI | p value |
| In non-smokers - N = 33 | Linear regression R² = 0.5779 | | |
| MM | 19.40 | 9.67-29.12 | <0.001 |
| Asbestos | 6.67 | −1.31-14.64 | 0.098 |
| Sex | 3.91 | −3.42-11.24 | 0.284 |
| constant | −10.60 | −27.28-6.08 | 0.204 |
| In smokers - N = 25 | Linear regression R² = 0.6809 | | |
| MM | 28.03 | 19.54-36.51 | <0.001 |
| Sex | −16.32 | −29.36-−3.28 | 0.017 |
| constant | 26.58 | 12.46-40-71 | 0.001 |

TABLE 4.2
Multivariate models for Hyper-acetylated HMGB1

| Hyper-acetylated HMGB1 | | | |
|---|---|---|---|
| Multivariate analysis | | | |
| Patients with known | Coef. | 95% CI | p value |
| smoking status-N = 58 | Linear regression R² = 0.7290 | | |
| MM | 19.13 | 15.72-22.53 | <0.001 |
| Smoking status | 1.23 | −1.96-4.41 | 0.443 |
| constant | −1.15 | −5.73-3.43 | 0.617 |

TABLE 4.3
Multivariate models for Hypo-acetylated HMGB1

| Hypo-acetylated HMGB1 | | | |
|---|---|---|---|
| Multivariate analysis | | | |
| Patients with known smoking | Coef. | 95% CI | p value |
| status - N = 58 | Linear regression R² = 0.5471 | | |
| Asbestos exposure | 5.22 | 1.85-8.58 | 0.003 |
| MM | 3.53 | 0.75-6.32 | 0.014 |
| Smoking status | 3.22 | 0.11-6.34 | 0.043 |
| constant | −7.30 | −11.69-−2.92 | 0.002 |

TABLE 4.4
Multivariate models for Fully reduced HMGB1

| Fully reduced HMGB1 | | | |
|---|---|---|---|
| Multivariate analysis | | | |
| Patients with known smoking | Coef. | 95% CI | p value |
| status - N = 58 | Linear regression R² = 0.4046 | | |
| MM | 6.42 | 2.85-9.99 | 0.001 |
| Smoking status | 2.48 | −1.21-6.17 | 0.183 |
| Asbestos exposure | 2.14 | −2.27-6.56 | 0.334 |
| Sex | −1.61 | −5.20-1.97 | 0.371 |
| Age | −1.11 | −4.67-2.45 | 0.534 |
| constant | 0.71 | −7.78-9.20 | 0.868 |

TABLE 4.5
Multivariate models for Percentage of hyper-acetylated HMGB1

| Percentage of hyper-acetylated HMGB1 | | | |
|---|---|---|---|
| Multivariate analysis | | | |
| Patients with known smoking | Coef. | 95% CI | p value |
| status - N = 58 | Linear regression R² = 0.6198 | | |
| MM | 0.53 | 0.42-0.64 | <0.001 |
| Asbestos exposure | −0.27 | −0.37-−0.16 | <0.001 |
| constant | 0.63 | 0.46-0.80 | <0.001 |

TABLE 4.6
Multivariate models for Disulfide HMGB1

| Disulfide HMGB1 | | | |
|---|---|---|---|
| Multivariate analysis | | | |
| Patients with known smoking | Coef. | 95% CI | p value |
| status - N = 58 | Linear regression R² = 0.6944 | | |
| MM | 19.96 | 16.46-23.45 | <0.001 |
| constant | 0.00 | −1.95-1.95 | <0.001 |

MM: malignant mesothelioma
All R² are very significant with a p-value <0.0001

### Example 4: Diagnosis of Malignant Mesothelioma

A method for diagnosing malignant mesothelioma is accordance with some embodiments herein is performed. A biological sample comprising serum of a subject at risk for malignant mesothelioma is provided. The serum sample is contacted with a mouse monoclonal antibody that binds specifically to hyper-acetylated HMGB1. The serum sample is contacted with a mouse monoclonal antibody that binds specifically to disulfide HMGB1 (the two antibody reactions are spatially separated). A secondary (goat anti-mouse) antibody that binds to the mouse primary antibodies is also contacted with the sample. The levels of antibodies that bind to hyper-acetylated HMGB1 and disulfide HMGB1 in the serum sample of the subject are detected. The levels of bound antibody (from which levels of hyper-acetylated HMGB1 and disulfide HMGB1 in the sample can be inferred) are then compared to a predetermined level. The bound levels of bound hyper-acetylated HMGB1 antibody and bound hyper-acetylated disulfide HMGB1 antibody are above the respective predetermined levels, and indicate that the subject has malignant mesothelioma. Subsequent appropriate treatments for malignant mesothelioma are then prescribed for the subject.

### Example 5: Differentiating Between Malignant Mesothelioma and Asbestos Exposure

A method for differentiating between malignant mesothelioma and asbestos exposure in a subject in accordance with some embodiments herein is performed. A lateral flow assay kit is provided, comprising an antibody that binds specifically to hyper-acetylated HMGB1 and an antibody that binds specifically to fully reduced HMGB1. A biological sample comprising serum from a subject suspected of having malignant mesothelioma is provided. The serum sample is contacted with the antibodies from the kit. The level of antibody bound to each of the two isoforms of HMGB1 (hyper-acetylated and disulfide) is detected via the lateral flow assay and compared to a predetermined level. The level of antibody bound to hyper-acetylated HMGB1 is greater than a predetermined level, and the level of antibody bound to fully reduced HMGB1 is greater than a predetermined level and indicates a presence of malignant mesothelioma in the subject rather than asbestos exposure. An appropriate therapy regiment for malignant mesothelioma is prescribed for the subject.

### Example 6: Differentiating Between Malignant Mesothelioma and Asbestos Exposure

A method for differentiating between malignant mesothelioma and asbestos exposure in a subject in accordance with some embodiments herein is provided. A biological sample comprising serum from a subject suspected of having malignant mesothelioma is provided. Levels of HMGB1 isoforms in the sample are detected by Electrospray Ionization Liquid Chromatography Mass Spectrometry. 10 ng/mL of disulfide HMGB1 are detected in the serum of the subject. This level of disulfide HMGB1 is greater than a predetermined level of 5 ng/mL. The subject is diagnosed as having malignant mesothelioma. An appropriate therapy regiment for malignant mesothelioma is prescribed for the subject.

### Example 7: Levels of HMGB1 Isoforms in the Serum of Various Subjects

Serum levels of HMGB1 isoforms for various patients were determined by Electrospray Ionization Liquid Chromatography Mass Spectrometry (ESI-LC-MS). The levels of each isoform are shown for healthy controls (Tables 7.1-7.2), mesothelioma patients (Tables 7.3-7.4), and asbestos-exposed individuals (Table 7.5-7.6). Levels of Total HMGB1, Hyper-acetylated HMGB1 and Hypo-acetylated HMGB1 are shown in ng/mL. Levels of fully reduced HMGB1 and Disulfide HMGB1 are shown as fold increase above lower limit of detection.

TABLE 7.1
Characteristics of healthy controls

| | | | SMOKING | | | |
|---|---|---|---|---|---|---|
| | | | PACK | CURRENT | | |
| ID | AGE | SEX | YEARS | SMOKING | ASBESTOS | MM |
| 01 | 38 | M | 0 | NO | NO | NO |
| 02 | 42 | F | 0 | NO | NO | NO |
| 03 | 21 | M | 0 | NO | NO | NO |
| 04 | 36 | F | 0 | NO | NO | NO |
| 05 | 44 | M | 0 | NO | NO | NO |
| 06 | 26 | M | 0 | NO | NO | NO |
| 07 | 56 | F | 0 | NO | NO | NO |
| 08 | 27 | M | 0 | NO | NO | NO |
| 09 | 37 | M | 0 | NO | NO | NO |
| 010 | 41 | M | 0 | NO | NO | NO |
| 011 | 49 | M | 0 | NO | NO | NO |
| 012 | 41 | M | 0 | NO | NO | NO |
| 013 | 25 | F | 0 | NO | NO | NO |
| 014 | 64 | F | 0 | NO | NO | NO |
| 015 | 29 | F | 0 | NO | NO | NO |
| 016 | 47 | M | 0 | NO | NO | NO |
| 017 | 23 | F | 0 | NO | NO | NO |
| 018 | 49 | M | 0 | NO | NO | NO |
| 019 | 41 | M | 0 | NO | NO | NO |
| 020 | 33 | F | 0 | NO | NO | NO |

TABLE 7.2
Levels of HMGB1 isoforms in healthy controls

| | SMOKING | | | Total | Hyperac | Hypoac | Fully reduced | Disulfide |
|---|---|---|---|---|---|---|---|---|
| | PACK | | | HMGB1 | HMGB1 | HMGB1 | HMGB | HMGB1 |
| ID | YEARS | ASBESTOS | MM | (ng/ml) | (ng/ml) | (ng/mL) | (FI-AUC-LLD) | (FI-AUC-LLD) |
| 01 | 0 | NO | NO | 1.1 | 0.5 | 0.6 | 1.3 | ND |
| 02 | 0 | NO | NO | 2.6 | 0.6 | 2 | 3.3 | ND |
| 03 | 0 | NO | NO | 1.9 | 0.3 | 1.6 | 3.1 | ND |
| 04 | 0 | NO | NO | 0.8 | 0.7 | 0.1 | 1.8 | ND |
| 05 | 0 | NO | NO | 2.3 | 0.8 | 1.5 | 2.8 | ND |
| 06 | 0 | NO | NO | 0.87 | 0.4 | 0.47 | 1.1 | ND |
| 07 | 0 | NO | NO | 0.81 | 0.1 | 0.71 | 1.1 | ND |
| 08 | 0 | NO | NO | 1.78 | 0.1 | 1.68 | 2.6 | ND |
| 09 | 0 | NO | NO | 0.32 | 0.2 | 0.12 | 1.2 | ND |
| 010 | 0 | NO | NO | 2.01 | 0.5 | 1.512 | 1.7 | ND |
| 011 | 0 | NO | NO | 2.29 | 0.9 | 1.388 | 1.9 | ND |
| 012 | 0 | NO | NO | 0.62 | 0.7 | −0.084 | 1.4 | ND |
| 013 | 0 | NO | NO | 1.41 | 0.6 | 0.808 | 1.2 | ND |
| 014 | 0 | NO | NO | 1.32 | 0.4 | 0.92 | 1.5 | ND |
| 015 | 0 | NO | NO | 1.76 | 0.3 | 1.46 | 1.5 | ND |
| 016 | 0 | NO | NO | 1.32 | 0.2 | 1.12 | 1.3 | ND |
| 017 | 0 | NO | NO | 1.98 | 0.6 | 1.38 | 1.4 | ND |
| 018 | 0 | NO | NO | 1.23 | 0.3 | 0.932 | 2.1 | ND |
| 019 | 0 | NO | NO | 2.36 | 0.5 | 1.864 | 3.2 | ND |
| 020 | 0 | NO | NO | 1.41 | 0.7 | 0.708 | 1.8 | ND |

TABLE 7.3
Characteristics of mesothelioma patients

| | | | SMOKING | | | |
|---|---|---|---|---|---|---|
| | | | PACK | CURRENT | | |
| ID | AGE | SEX | YEARS | SMOKING | ASBESTOS | MM |
| 1107S | 61 | F | 0 | NO | <20 years | YES |
| 1336A | 66 | F | 0 | NO | <20 years | YES |
| 939S | 50 | F | 15 | NO | NO | YES |
| 851S | 58 | F | 1 | NO | <20 years | YES |
| 1189S | 76 | M | 30 | NO | <20 years | YES |
| 1283 | 71 | M | 30 | NO | <20 years | YES |
| 1306 | 73 | F | 5 | NO | <20 years | YES |
| 1336 | 66 | F | 0 | NO | <20 years | YES |
| 1338 | 80 | M | 0 | NO | <20 years | YES |
| 1353 | 81 | M | 15 | NO | <20 years | YES |
| 1356 | 73 | M | UNK | UNK | ≧20 years | YES |
| 1363 | 64 | M | 20 | NO | <20 years | YES |
| 1373 | 55 | M | 0 | NO | <20 years | YES |
| 1396 | 59 | M | 30 | NO | ≧20 years | YES |
| 1419 | 25 | F | 0 | NO | NO | YES |
| 1621 | 89 | M | 0 | NO | ≧20 years | YES |
| 1633 | 77 | M | 10 | NO | ≧20 years | YES |
| 1672 | 75 | M | 30 | NO | ≧20 years | YES |
| 1744 | 59 | M | 7 | NO | <20 years | YES |
| 1755 | 74 | M | 30 | NO | <20 years | YES |

TABLE 7.4
Levels of HMGB1 in mesothelioma patients

| | | Total | Hyperac- | Hypoac- | Fully reduced | Disulfide |
|---|---|---|---|---|---|---|
| | | HMGB1 | HMGB1 | HMGB1 | HMGB | HMGB1 |
| ID | MM | (ng/ml) | (ng/ml) | (ng/mL) | (FI-AUC-LLD) | (FI-AUC-LLD) |
| 1107S | YES | 59.9 | 39.8 | 20.1 | 17.9 | 41.3 |
| 1336S | YES | 16.4 | 12.4 | 4 | 6.6 | 11.5 |
| 939S | YES | 23.2 | 16.3 | 6.9 | 5.9 | 11.6 |
| 851S | YES | 16.6 | 10.7 | 5.9 | 4.8 | 16.7 |
| 1189S | YES | 36.1 | 21.9 | 14.2 | 10.3 | 23.4 |
| 1283 | YES | 63.4 | 40.2 | 23.2 | 17.1 | 30.6 |
| 1306 | YES | 26.1 | 17.4 | 8.7 | 8.3 | 20.5 |
| 1336 | YES | 20.5 | 13.4 | 7.1 | 6.1 | 10.5 |
| 1338 | YES | 11.4 | 6.1 | 5.3 | 3.4 | 5.4 |
| 1353 | YES | 45.7 | 27.9 | 17.8 | 11.4 | 44.1 |
| 1356 | YES | 7.8 | 5.9 | 1.9 | 1.2 | 5.1 |
| 1363 | YES | 36.6 | 20.7 | 15.9 | 9.8 | 20.3 |
| 1373 | YES | 44.1 | 26.8 | 17.3 | 20.6 | 30.8 |
| 1396 | YES | 36.3 | 20.9 | 15.4 | 9.4 | 25.6 |
| 1419 | YES | 23.9 | 17.3 | 6.6 | 7.1 | 20.4 |
| 1621 | YES | 3.6 | 2.4 | 1.2 | 0.8 | 6.5 |
| 1633 | YES | 27.3 | 19.7 | 7.6 | 10.7 | 10.4 |
| 1672 | YES | 27.8 | 18.7 | 9.1 | 8.3 | 11.5 |
| 1744 | YES | 15.8 | 10.3 | 5.5 | 9.7 | 18.6 |
| 1755 | YES | 55.6 | 30.4 | 25.2 | 40.5 | 10.5 |

TABLE 7.5
Characteristics of asbestos-exposed individuals

| | | | SMOKING | | | |
|---|---|---|---|---|---|---|
| | | | PACK | CURRENT | | |
| ID | AGE | SEX | YEARS | SMOKING | ASBESTOS | MM |
| 1-S | 42 | M | 5 | NO | <20 years | NO |
| 10-S | 56 | M | 11.1 | NO | ≧20 years | NO |
| 2-S | 49 | M | 0 | NO | <20 years | NO |
| 6-S | 65 | M | 64 | NO | ≧20 years | NO |
| 14-S | 47 | M | 0 | NO | ≧20 years | NO |
| 15 | 56 | M | 5 | NO | ≧20 years | NO |
| 16 | 62 | M | 12 | NO | ≧20 years | NO |
| 17 | 52 | M | 0 | NO | ≧20 years | NO |
| 18 | 44 | M | 29 | YES | ≧20 years | NO |
| 19 | 56 | M | 0 | NO | ≧20 years | NO |
| 20 | 58 | M | 0 | NO | ≧20 years | NO |
| 21 | 55 | M | 0.75 | NO | ≧20 years | NO |
| 22 | 53 | M | 13.6 | NO | <20 years | NO |
| 23 | 70 | M | 12 | NO | ≧20 years | NO |
| 24 | 56 | M | 4.5 | NO | ≧20 years | NO |
| 25 | 63 | M | 54 | NO | ≧20 years | NO |
| 26 | 55 | M | 0 | NO | <20 years | NO |
| 27 | 65 | M | 0 | NO | ≧20 years | NO |
| 28 | 42 | M | 5 | NO | <20 years | NO |
| 29 | 70 | M | 1.8 | NO | ≧20 years | NO |

TABLE 7.6
Levels of HMGB1 in asbestos-exposed individuals

| | | | Total | Hyperac- | Hypoac- | Fully reduced | Disulfide |
|---|---|---|---|---|---|---|---|
| | | | HMGB1 | HMGB1 | HMGB1 | HMGB | HMGB1 |
| ID | ASBESTOS | MM | (ng/ml) | (ng/ml) | (ng/mL) | (FI-AUC-LLD) | (FI-AUC-LLD) |
| 1-S | <20 years | NO | 11.1 | 0.4 | 10.7 | 6.7 | ND |
| 10-S | ≧20 years | NO | 15.7 | 0.2 | 15.5 | 9.2 | ND |
| 2-S | <20 years | NO | 4.9 | 0.3 | 4.6 | 2.8 | ND |
| 6-S | ≧20 years | NO | 13.8 | 0.4 | 13.4 | 8.1 | ND |
| 14-S | ≧20 years | NO | 11.4 | 0.6 | 10.8 | 6.1 | ND |
| 15 | ≧20 years | NO | 6.4 | 0.1 | 6.3 | 4.2 | ND |
| 16 | ≧20 years | NO | 3.5 | 0.8 | 2.7 | 1.3 | ND |
| 17 | ≧20 years | NO | 9.9 | 0.1 | 9.8 | 6.5 | ND |
| 18 | ≧20 years | NO | 12.5 | 0.1 | 12.4 | 10.5 | ND |
| 19 | ≧20 years | NO | 5.6 | 0.3 | 5.3 | 5.9 | ND |
| 20 | ≧20 years | NO | 10.4 | 0.3 | 10.1 | 4.7 | ND |
| 21 | ≧20 years | NO | 11.7 | 1.9 | 9.8 | 8.4 | ND |
| 22 | <20 years | NO | 14.2 | 0.6 | 13.6 | 6.4 | ND |
| 23 | ≧20 years | NO | 8.3 | 0.4 | 7.9 | 6.9 | ND |
| 24 | ≧20 years | NO | 10.5 | 0.2 | 10.3 | 7.4 | ND |
| 25 | ≧20 years | NO | 13.6 | 1.2 | 12.4 | 8.5 | ND |
| 26 | <20 years | NO | 3.8 | 0.3 | 3.5 | 2.6 | ND |
| 27 | ≧20 years | NO | 1.1 | 0.7 | 0.4 | 0.5 | ND |
| 28 | <20 years | NO | 6.4 | 0.3 | 6.1 | 4.7 | ND |
| 29 | ≧20 years | NO | 5.7 | 0.4 | 5.3 | 3.3 | ND |

As such, levels of one or more HMGB1 isoforms can be detecting in individual patients in accordance with some embodiments herein. Additionally, it is contemplated that particular levels of particular HMGB1 isoforms can provide signatures characteristics of various populations.

### Example 8: HMGB1 Levels in Supernatants of HM and MM Cells in Tissue Culture

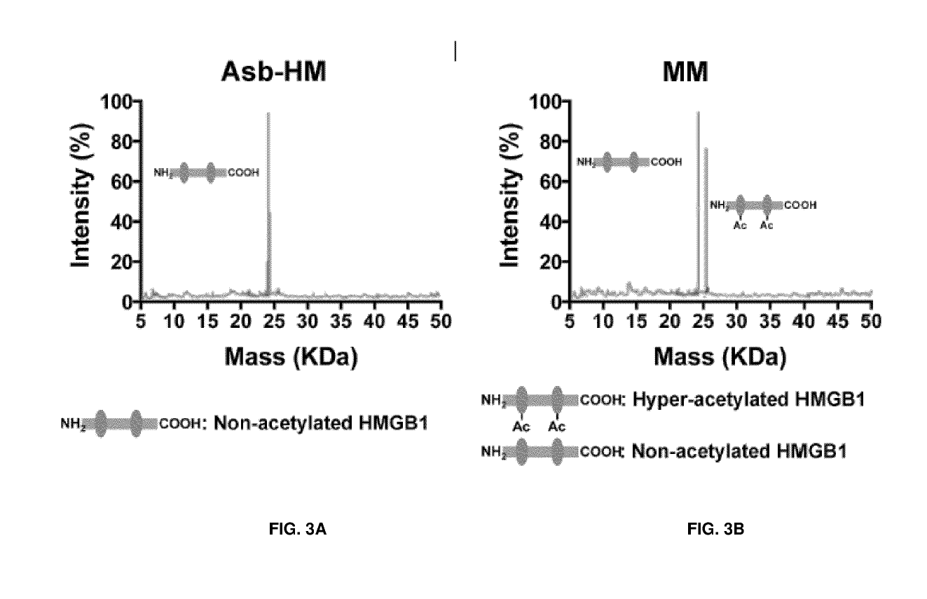

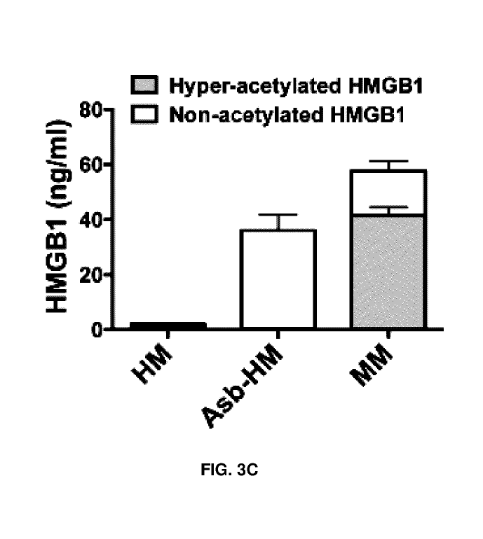

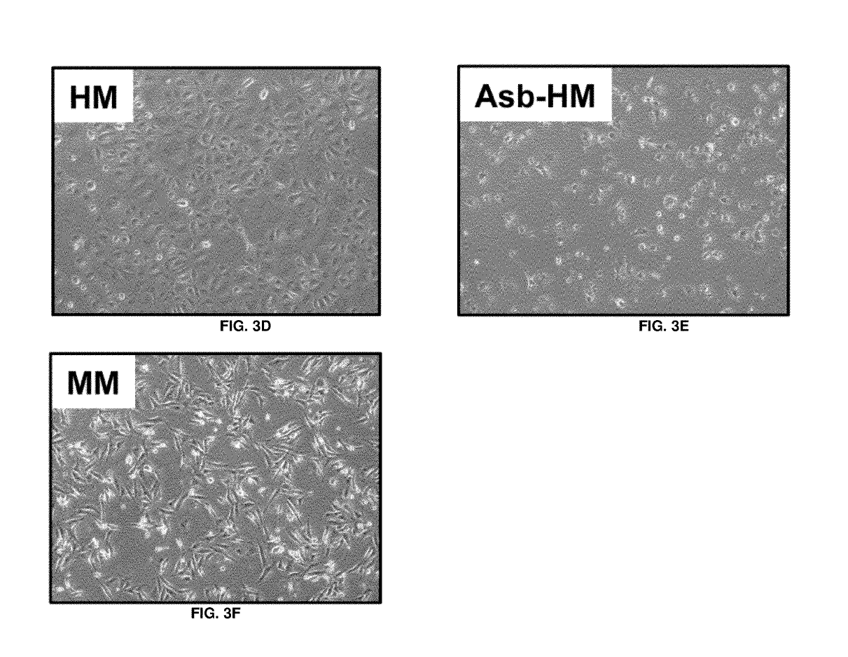

HMGB1 levels from concentrated supernatant of HM and MM cells in tissue culture were measured. Unexposed HM cells did not release detectable HMGB1 into the extracellular space (FIG. 3C). Only hypo-acetylated HMGB1 was detected in asbestos-exposed HM cell supernatant (about 37 ng/mL), and both hypo-acetylated HMGB1 (about 58 ng/mL) and hyper-acetylated HMGB1 (about 40 ng/mL) were measured in supernatants from MM cells (FIG. 3C).

When HM cells are exposed to 5 μg/cm² of crocidolite asbestos, ˜60-70% of them undergo programmed necrosis within 48 hours. In the supernatant of HM cells exposed to asbestos (Asb-HM), high levels of hypo-acetylated HMGB1 were consistently detected, as expected from cells undergoing necrosis (FIGS. 3A, 3C). In the supernatant of MM cells, actively secreted hyper-acetylated HMGB1, as well as hypo-acetylated HMGB1, were detected (FIGS. 3B, 3C). Without being limited by any theory, it is contemplated that the hypo-acetylated HMGB1 was likely released by a fraction of MM cells undergoing necrosis, as they are grown in serum-free tissue culture condition. Supernatants from MM cells showed higher levels of total (hypo-acetylated and hyper-acetylated) HMGB1 with a prevalence of the hyper-acetylated isoform, compared to the supernatants from HM exposed to asbestos that contained prevalently the hypo-acetylated isoform (FIG. 3C). The presence of hypo-acetylated HMGB1 in asbestos-exposed HM was associated with significant cell death with loss of the classic HM cobblestone appearance (FIG. 3D).

As such, measurements of levels of hypo-acetylated and/or hyper-acetylated HMGB1 in accordance with kits and/or methods of some embodiments herein can differentiate malignant mesothelioma cells from (i) asbestos-exposed cells and from (ii) cells that are neither asbestos-exposed nor have malignant mesothelioma. Additionally, measurements of total levels HMGB1 in accordance with kits and/or methods in accordance with some embodiments herein can differentiate malignant mesothelioma cells from (i) asbestos-exposed cells and from (ii) cells that are neither asbestos-exposes nor have malignant mesothelioma.

### Example 9: Discriminating Individuals with Asbestos-Exposure and/or MM from Healthy Controls Using HMGB1

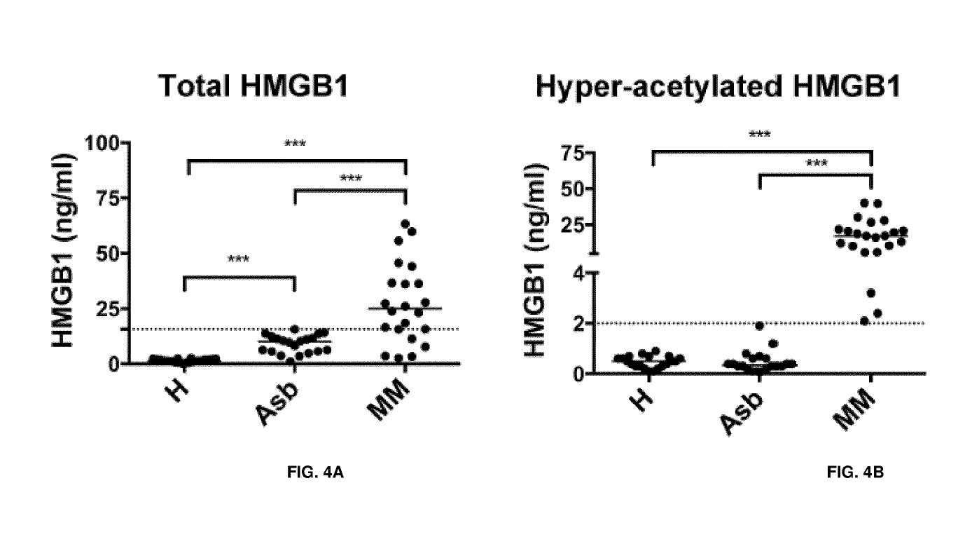

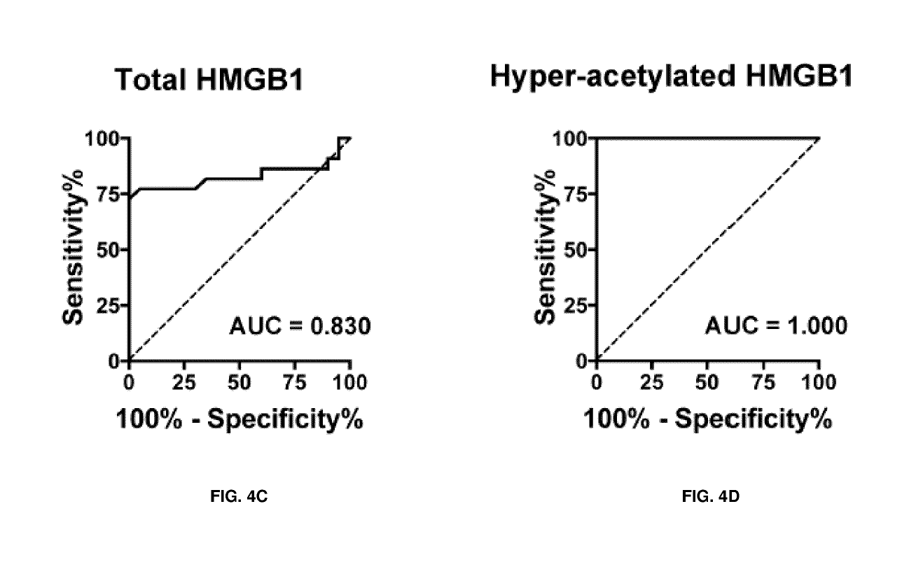

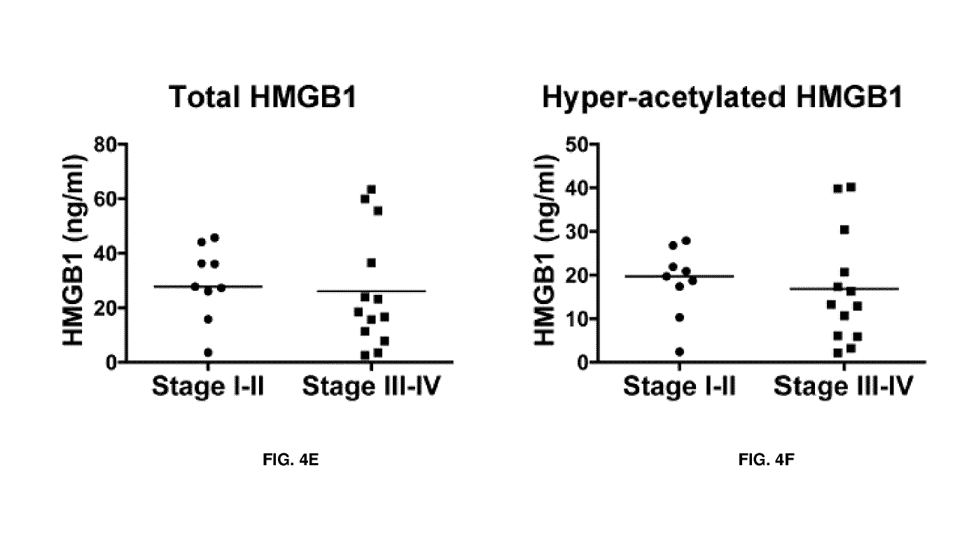

In another experiment, total and isoform-specific HMGB1 in the sera from all three cohorts (asbestos-unexposed healthy controls, MM patients, and individuals with asbestos exposure) were measured. In the (unexposed) healthy control cohort, total levels of HMGB1 detected were very low (1.4, 0.8-2.2 ng/ml). Total HMGB1 serum levels were significantly higher in asbestos-exposed individuals (10.2, 5.7-12.1 ng/ml) compared to the levels in unexposed controls (P<0.001). MM patients had the highest levels of total HMGB1 (25.0, 15.7-36.6 ng/ml) when compared to either other group (P<0.001) (FIG. 4A). The total levels of HMGB1 measured with a commercially available ELISA kit and with MS as described herein were very similar (R²=0.92, P<0.0001), corroborating the reliability of the measurement of the approach. The levels of hyper-acetylated HMGB1 were very low in both the healthy controls (0.5, 0.3-0.7 ng/ml) and asbestos-exposed individuals (0.4, 0.3-0.6 ng/ml). MM patients showed significantly higher levels of hyper-acetylated HMGB1 (17.4, 10.3-21.9 ng/ml) compared to either other group (P<0.001) (FIG. 4B). Overall, hyper-acetylated HMGB1 comprised ˜10% of the total HMGB1 in the sera of asbestos-exposed individuals, and ˜67% of the total HMGB1 in the sera of MM patients.

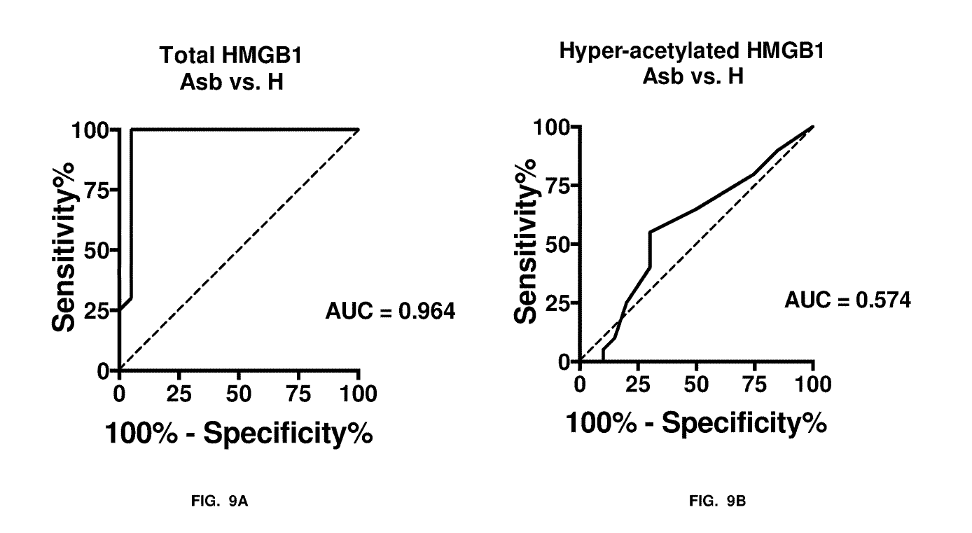

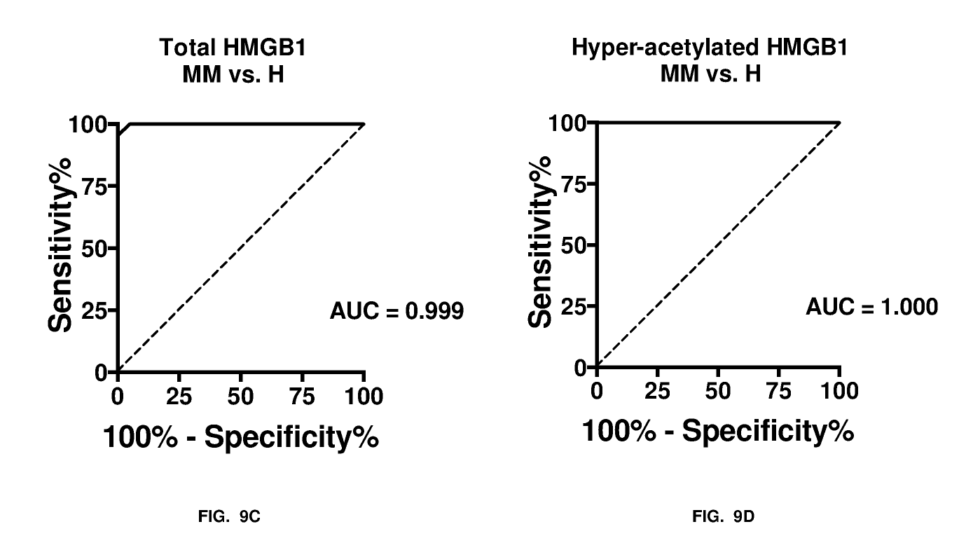

The sensitivity and specificity of total and hyper-acetylated HMGB1 as potential biomarkers to discriminate MM patients from asbestos-exposed individuals and healthy controls were assessed. Both, total and hyper-acetylated HMGB1, showed exceptional accuracy in discriminating MM patients from healthy controls with a receiver operating characteristic (ROC) area under the curve (AUC) of 0.999 (95% CI 0.994-1.000) and 1.000 (95% CI 1.000-1.000), respectively. Comparing asbestos-exposed individuals to healthy controls, the AUC of total and hyper-acetylated HMGB1 were 0.964 (95% CI 0.893-1.000) and 0.574 (95% CI 0.392-0.756), respectively (FIGS. 9A-9D; Tables 8, 9). These data indicate that total HMGB1 in accordance with methods and/or kits of some embodiments herein is a reliable biomarker to discriminate individuals with asbestos-exposure and/or MM from healthy controls.

TABLE 8
AUC of total and hyper-acetylated HMGB1 comparing healthy
controls, asbestos-exposed individuals, and MM patients.

| | Total HMGB1 | | Hyper-acetylated HMGB1 | |
|---|---|---|---|---|
| | AUC | Cut-off | AUC | Cut-off |
| | (95% CI) | (ng/ml) | (95% CI) | (ng/ml) |
| MM vs. Healthy | 0.999 (0.994-1.000) | 3.00 | 1.000 (1.000-1.000) | 1.50 |
| Asbestos-exposed vs. | 0.964 (0.893-1.000) | 3.05 | 0.574 (0.392-0.756) | 0.45 |
| Healthy | | | | |
| MM vs. Asbestos- | 0.830 (0.687-0.972) | 15.75 | 1.000 (1.000-1.000) | 2.00 |
| exposed | | | | |

TABLE 9
Full demographic characterization of individuals involved in the study.

| | | | | Profes- | | | | | Hyper- | | | |
|---|---|---|---|---|---|---|---|---|---|---|---|---|
| | | | | sional | | | | Total | acetylated | | | |
| | | | | Asbestos | | | Pleural | HMGB1 | HMGB1 | Fibulin-3 | OPN | MRP |
| ID | Age | Sex | Smoking | Exposure | Cancer | Stage | Effusions | (ng/mL) | (ng/mL) | (ng/mL) | (ng/mL) | (ng/mL) |
| H1 | 38 | M | NO | NO | NO | | NO | 1.1 | 0.5 | | | |
| H2 | 42 | F | NO | NO | NO | | NO | 2.6 | 0.6 | | | |
| H3 | 21 | M | NO | NO | NO | | NO | 1.9 | 0.3 | | | |
| H4 | 36 | F | NO | NO | NO | | NO | 0.8 | 0.7 | | | |
| H5 | 44 | M | NO | NO | NO | | NO | 2.3 | 0.8 | | | |
| H6 | 26 | M | NO | NO | NO | | NO | 0.87 | 0.4 | | | |
| H7 | 56 | F | NO | NO | NO | | NO | 0.81 | 0.1 | | | |
| H8 | 27 | M | NO | NO | NO | | NO | 1.78 | 0.1 | | | |
| H9 | 37 | M | NO | NO | NO | | NO | 0.32 | 0.2 | | | |
| H10 | 41 | M | NO | NO | NO | | NO | 2.01 | 0.5 | | | |
| H11 | 49 | M | NO | NO | NO | | NO | 2.29 | 0.9 | | | |
| H12 | 41 | M | NO | NO | NO | | NO | 0.62 | 0.7 | | | |
| H13 | 25 | F | NO | NO | NO | | NO | 1.41 | 0.6 | | | |
| H14 | 64 | F | NO | NO | NO | | NO | 1.32 | 0.4 | | | |
| H15 | 29 | F | NO | NO | NO | | NO | 1.76 | 0.3 | | | |
| H16 | 47 | M | NO | NO | NO | | NO | 1.32 | 0.2 | | | |
| H17 | 23 | F | NO | NO | NO | | NO | 1.98 | 0.6 | | | |
| H18 | 49 | M | NO | NO | NO | | NO | 1.23 | 0.3 | | | |
| H19 | 41 | M | NO | NO | NO | | NO | 2.36 | 0.5 | | | |
| H20 | 33 | F | NO | NO | NO | | NO | 1.41 | 0.7 | | | |
| Asb1 | 42 | M | YES | <20 years | NO | | NO | 11.1 | 0.4 | 21.4756 | 34.96 | 12.50882 |
| Asb2 | 56 | M | YES | ≧20 years | NO | | NO | 15.7 | 0.2 | 13.2877 | 21.399725 | 23.8244 |
| Asb3 | 49 | M | NO | <20 years | NO | | NO | 4.9 | 0.3 | 40.1556 | 53.95 | 16.31582 |
| Asb4 | 65 | M | YES | ≧20 years | NO | | NO | 13.8 | 0.4 | 51.8484 | 17.590575 | 18.53108 |
| Asb5 | 47 | M | NO | ≧20 years | NO | | NO | 11.4 | 0.6 | 22.8123 | 17.49605 | 19.13404 |
| Asb6 | 56 | M | YES | ≧20 years | NO | | NO | 6.4 | 0.1 | 18.5905 | 37.324 | 17.02064 |
| Asb7 | 62 | M | YES | ≧20 years | NO | | NO | 3.5 | 0.8 | 14.6779 | 35.54375 | 9.52488 |
| Asb8 | 52 | M | NO | ≧20 years | NO | | NO | 9.9 | 0.1 | 21.123 | 8.80555 | 11.65856 |
| Asb9 | 44 | M | YES | ≧20 years | NO | | NO | 12.5 | 0.1 | 16.8203 | 44.3125 | 30.5134 |
| Asb10 | 56 | M | NO | ≧20 years | NO | | NO | 5.6 | 0.3 | 17.8959 | 29.4095 | 29.0932 |
| Asb11 | 58 | M | NO | ≧20 years | NO | | NO | 10.4 | 0.3 | 25.5682 | 56.24175 | 16.6412 |
| Asb12 | 55 | M | YES | ≧20 years | NO | | NO | 11.7 | 1.9 | 15.004 | 12.753925 | 15.82444 |
| Asb13 | 53 | M | YES | <20 years | NO | | NO | 14.2 | 0.6 | 56.3542 | 27.009 | 13.547 |
| Asb14 | 70 | M | YES | ≧20 years | NO | | NO | 8.3 | 0.4 | 5.25956 | 3.9168 | 19.66094 |
| Asb15 | 56 | M | YES | ≧20 years | NO | | NO | 10.5 | 0.2 | 2.8734 | 36.77525 | 16.3464 |
| Asb16 | 63 | M | YES | ≧20 years | NO | | NO | 13.6 | 1.2 | 18.9485 | 2.669475 | 10.44004 |
| Asb17 | 55 | M | NO | <20 years | NO | | NO | 3.8 | 0.3 | 6.12838 | 35.12675 | 9.04916 |
| Asb18 | 65 | M | NO | ≧20 years | NO | | NO | 1.1 | 0.7 | 7.69722 | 10.9303 | 21.813 |
| Asb19 | 42 | M | YES | <20 years | NO | | NO | 6.4 | 0.3 | 3.31255 | 28.37825 | 17.5944 |
| Asb20 | 70 | M | YES | ≧20 years | NO | | NO | 5.7 | 0.4 | 3.00255 | 46.46775 | 9.05864 |
| MM1 | 61 | F | NO | <20 years | MM | 3 | YES | 59.9 | 39.8 | 46.2541 | 54.55 | 44.1934 |
| MM2 | 66 | F | NO | <20 years | MM | 3 | YES | 16.4 | 12.4 | 88.1786 | 76.4015 | 58.9668 |
| MM3 | 50 | F | YES | NO | MM | 4 | YES | 23.2 | 16.3 | 151.144 | 70.12875 | 108.1566 |
| MM4 | 58 | F | YES | <20 years | MM | 3 | YES | 16.6 | 10.7 | 111.936 | 77.84725 | 141.83 |
| MM5 | 76 | M | YES | <20 years | MM | 2 | YES | 36.1 | 21.9 | 49.8787 | 119.8525 | 45.6438 |
| MM6 | 71 | M | YES | <20 years | MM | 3 | YES | 63.4 | 40.2 | 36.7745 | 409.495 | 192.2712 |
| MM7 | 73 | F | YES | <20 years | MM | 2 | YES | 26.1 | 17.4 | 41.5634 | 56.46175 | 29.5894 |
| MM8 | 80 | M | NO | <20 years | MM | 3 | YES | 11.4 | 6.1 | 83.8305 | 155.24525 | 22.6902 |
| MM9 | 81 | M | YES | <20 years | MM | 2 | YES | 45.7 | 27.9 | 37.3635 | 150.93375 | 34.05 |
| MM10 | 73 | M | | ≧20 years | MM | 3 | YES | 7.8 | 5.9 | 56.2035 | 75.0035 | 92.116 |
| MM11 | 64 | M | YES | <20 years | MM | 3 | YES | 36.6 | 20.7 | 128.071 | 139.99275 | 220.984 |
| MM12 | 55 | M | NO | <20 years | MM | 2 | YES | 44.1 | 26.8 | 38.8566 | 44.186 | 17.80812 |
| MM13 | 59 | M | YES | ≧20 years | MM | 1 | YES | 36.3 | 20.9 | 87.6252 | 33.795 | 25.8422 |
| MM14 | 25 | F | NO | NO | MM | 3 | YES | 23.9 | 17.3 | 200.554 | 84.887 | 59.3842 |
| MM15 | 89 | M | NO | ≧20 years | MM | 1 | YES | 3.6 | 2.4 | 60.8523 | 95.50625 | 30.8464 |
| MM16 | 77 | M | YES | ≧20 years | MM | 2 | YES | 27.3 | 19.7 | 119.358 | 172.11375 | 27.619 |
| MM17 | 75 | M | YES | ≧20 years | MM | 1 | YES | 27.8 | 18.7 | 101.575 | 60.24375 | 42.1062 |
| MM18 | 59 | M | YES | <20 years | MM | 2 | YES | 15.8 | 10.3 | 45.0645 | 45.1065 | 66.6734 |
| MM19 | 74 | M | YES | <20 years | MM | 3 | YES | 55.6 | 30.4 | 59.2125 | 106.44025 | 43.5086 |
| MM20 | 56 | M | NO | | MM | 3 | YES | 15.7 | 13.3 | 240.10325 | 240.10325 | 106.305 |
| MM21 | 65 | F | NO | | MM | 3 | YES | 3.4 | 3.2 | 145.10725 | 145.10725 | 174.222 |
| MM22 | 65 | M | YES | | MM | 4 | YES | 2.6 | 2.1 | 164.38425 | 164.38425 | 12.8443 |
| Ben1 | M | 67 | YES | | NO | | YES | 6.5 | 5.2 | 63.3642 | 97.84775 | 16.4524 |
| Ben2 | F | 78 | | | NO | | YES | 10.4 | 8.1 | 39.4971 | 255.8175 | 102.003 |
| Mal1 | F | 82 | YES | | LYMPHOMA | | YES | 12.6 | 11.3 | 41.9381 | 56.72775 | 34.028 |
| Mal2 | M | 86 | NO | | LYMPHOMA | | YES | 6.9 | 5.9 | 9.08033 | 99.64375 | 30.1432 |
| Mal3 | M | 18 | NO | | NSCLC | | YES | 1.5 | 1.1 | 0.2 | 69.33175 | 8.47577 |
| Ben3 | F | 43 | NO | | NO | | YES | 12.8 | 10.6 | 22.2148 | 75.675 | 10.4681 |
| Ben4 | M | 74 | YES | | NO | | YES | 10.6 | 8.9 | 21.2225 | 74.47075 | 21.3811 |
| Mal4 | M | 65 | NO | | NSCLC | | YES | 3.4 | 2.9 | 8.67527 | 121.3458 | 38.209 |
| Ben5 | F | 47 | NO | | NO | | YES | 4.8 | 3.9 | 30.4874 | 105.444 | 28.4325 |
| Ben6 | M | 84 | YES | | NO | | YES | 8.9 | 7.5 | 23.8493 | 56.364 | 35.5268 |
| Ben7 | M | 66 | YES | | NO | | YES | 6.4 | 5.1 | 39.0355 | 137.0808 | 25.9947 |
| Ben8 | M | 63 | YES | | NO | | YES | 3.3 | 2.6 | 40.6437 | 65.47375 | 21.3165 |
| Ben9 | M | 67 | YES | | NO | | YES | 5.5 | 4.1 | 18.6413 | 57.0265 | 23.9214 |
| Ben10 | F | 73 | YES | | NO | | YES | 6.4 | 5.7 | 2.4003 | 63.373 | 20.7841 |
| Mal5 | M | 65 | YES | | NSCLC | | YES | 17.8 | 15.7 | 15.1932 | 148.5098 | 22.1066 |
| Ben11 | F | 93 | NO | | NO | | YES | 8.7 | 6.9 | 53.3543 | 51.13475 | 20.9697 |
| Ben12 | F | 60 | YES | | NO | | YES | 4.5 | 3.4 | 26.2048 | 126.5385 | 18.7316 |
| Ben13 | M | 59 | NO | | NO | | YES | 3.4 | 2.9 | 19.0393 | 96.8095 | 49.0096 |
| Mal6 | F | 68 | | | BREAST | | YES | 6.6 | 5.7 | 9.27292 | 81.02375 | 27.3833 |
| Mal7 | F | 40 | NO | | BREAST | | YES | 1.4 | 1.3 | 63.2857 | 431.475 | 215.512 |
| Mal8 | F | 45 | | | CHOLANGIO | | YES | 7.7 | 5.9 | 64.4723 | 433.8425 | 23.0554 |
| Mal9 | M | 75 | NO | | MELANOMA | | YES | 5.1 | 4.2 | 23.0104 | 195.906 | 78.7056 |
| Mal10 | M | 62 | NO | | NSCLC | | YES | 10.2 | 9.1 | 32.9417 | 137.4518 | 26.3117 |
| Mal11 | M | 66 | YES | | SCLC | | YES | 12.4 | 11.6 | 56.1929 | 229.431 | 41.0153 |
| Mal12 | F | 77 | YES | | NSCLC | | YES | 5.1 | 4.8 | 25.2506 | 214.0833 | 31.3136 |
| Mal13 | F | 85 | NO | | SARCOMA | | YES | 6.7 | 5.4 | 22.7689 | 51.42775 | 26.1407 |
| Mal14 | M | 86 | NO | | LYMPHOMA | | YES | 2.3 | 1.9 | 11.4095 | 78.43475 | 25.5605 |
| Mal15 | F | 78 | YES | | NSCLC | | YES | 3.8 | 2.5 | 18.5142 | 66.14925 | 16.9944 |
| Mal16 | F | 79 | NO | | NSCLC | | YES | 9.8 | 7.9 | 0.2 | 59.29125 | 4.389 |
| Mal17 | M | 56 | NO | | NSCLC | | YES | 4.5 | 3.7 | 14.6286 | 31.606 | 22.6945 |
| Mal18 | M | 67 | YES | | NSCLC | | YES | 7.5 | 6.4 | 2.20185 | 82.04775 | 8.40764 |
| Mal19 | M | 49 | NO | | SFTP | | YES | 6.7 | 5.4 | 0.29129 | 53.919 | 32.2524 |
| Mal20 | F | 87 | NO | | LYMPHOMA | | YES | 8.7 | 6.4 | 15.7883 | 88.08575 | 10.9371 |
| Mal21 | M | 50 | | | SARCOMA | | YES | 11.3 | 9.1 | 1.44303 | 129.4805 | 19.6771 |
| Mal22 | F | 61 | NO | | UNKNOWN | | YES | 2.3 | 1.7 | 2.464 | 183.872 | 15.0376 |
| Mal23 | F | 63 | NO | | BREAST | | YES | 10.6 | 8.5 | 0.2 | 32.558 | 4.20473 |
| Mal24 | F | 67 | NO | | PANCREAS | | YES | 6.7 | 5.7 | 39.7719 | 155.856 | 27.5158 |
| Mal25 | M | 78 | YES | | EMCa | | YES | 5.9 | 4.7 | 0.2 | 93.71525 | 3.41595 |

Abbreviations for Table 8:
H: healthy individuals
Asb: asbestos-exposed individuals
MM: malignant mesothelioma patients
Ben: patients with benign pleural effusions
Mal: patients with malign non-MM pleural effusions
NSCLC: non small cell lung carcinoma
SCLC: small cell lung carcinoma
SFTP: solitary fibrous tumor of the pleura
EMCa: epithelial-myoepithelial carcinoma

Comparing MM patients and asbestos-exposed individuals, the AUC of total HMGB1 was 0.830 (95% CI 0.687-0.972). At specificity 100%, the sensitivity was 72.73% (for values>15.75 ng/ml, which also corresponded to the cut-off value); at sensitivity 100%, the specificity was 5%. (FIG. 4C) The AUC of hyper-acetylated HMGB1, when comparing MM patients to asbestos-exposed individuals, was 1.000 (95% CI 1.000-1.000), with cut-off value of 2.00 ng/ml. (FIG. 4D) These results indicate that hyper-acetylated HMGB1 is a sensitive and specific biomarker to discriminate MM patients from asbestos-exposed individuals.

The differences in serum levels of total HMGB1 and hyper-acetylated HMGB1 in MM patients in their early stage (Stage I-II) and late stage (Stage III-IV) were measured (FIGS. 4E, 4F). No significant difference was detected, suggesting that early lesions are also associated with increased HMGB1 levels and that hyper-acetylated HMGB1 may be a valuable screening tool for early detection of MM among asbestos-exposed cohorts.

As such, measurement of serum levels of hyper-acetylated HMGB1 and total HMGB1 in accordance with kits and/or methods of some embodiments herein can be useful for early detection of MM among asbestos-exposed cohorts.

### Example 10: Discriminating MM Patients and Patients with Pleural Effusions Due to Other Causes

Total and hyper-acetylated HMGB1 in MM patients and patients with pleural effusions due to other causes were measured. Additionally, sensitivity and specificity of total and hyper-acetylated HMGB1 were measured to discriminate MM patients from patients with pleural effusions due to other causes.

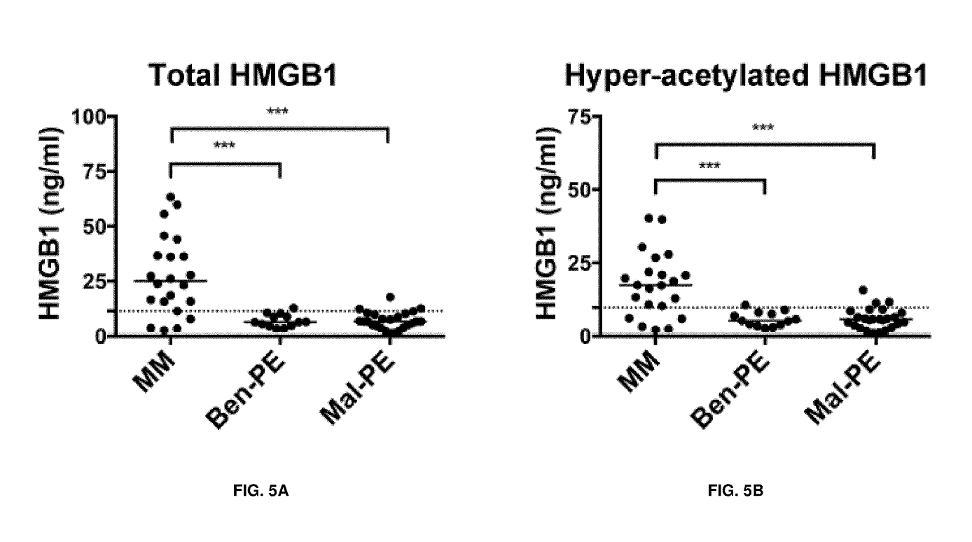

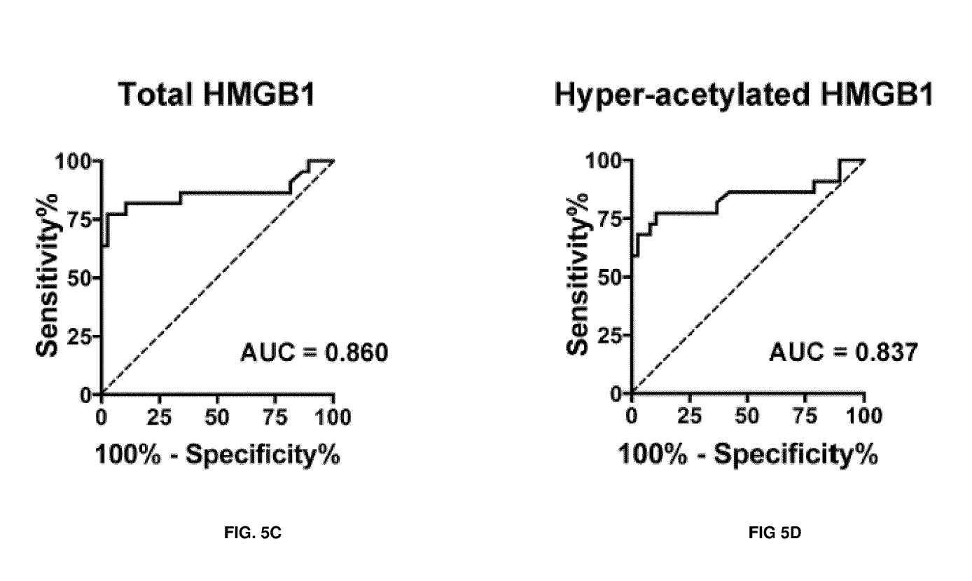

Thirteen serum samples from patients with cytologically benign pleural effusions and 25 serum samples from patients with pleural effusions due to non-MM malignancy were available for these studies. MM patients had significantly higher levels of total HMGB1 compared to patients with cytologically benign pleural effusions (6.4, 4.7-9.7 ng/ml; P<0.001) and malignant (non-MM) pleural effusions (6.7, 4.2-10.0 ng/ml; P<0.001) (FIG. 5A). Levels of hyper-acetylated HMGB1 were significantly higher in the sera from MM patients compared to sera from patients with benign pleural effusions (5.2, 3.7-7.8 ng/ml; P<0.001) or malignant (non-MM) pleural effusions (5.7, 3.3-8.2 ng/ml; P<0.001) (FIG. 5B).

The sensitivity and specificity of total and hyper-acetylated HMGB1 to discriminate MM patients from patients with pleural effusions due to other causes were also assessed. The AUC of total HMGB1 was 0.860 (95% CI 0.736-0.984) (FIG. 5C); at specificity 100%, the sensitivity was 63.64%; at sensitivity 100%, the specificity was 10.53%. The AUC for hyper-acetylated HMGB1 was 0.837 (95% CI 0.709-0.966) (FIG. 5D); at specificity 100%, the sensitivity was 59.09%; at sensitivity 100%, the specificity was 10.53%. Best cut-off values to discriminate MM patients from patients with benign or malignant non-MM pleural effusions were 11.35 ng/ml (sensitivity 81.82%, specificity 89.47%) and 9.70 ng/ml (sensitivity 77.27%, specificity 89.47%) respectively for total and hyper-acetylated HMGB1. These results indicate that levels of total and hyper-acetylated HMGB1 in accordance with kits and/or methods of some embodiments herein can be used to distinguish MM patients from patients with pleural effusions due to other causes.

Very low levels of hyper-acetylated HMGB1 (<2.00 ng/ml) were found both in asbestos-exposed individuals and healthy controls.

### Example 11: Levels of Other Possible MM Biomarkers

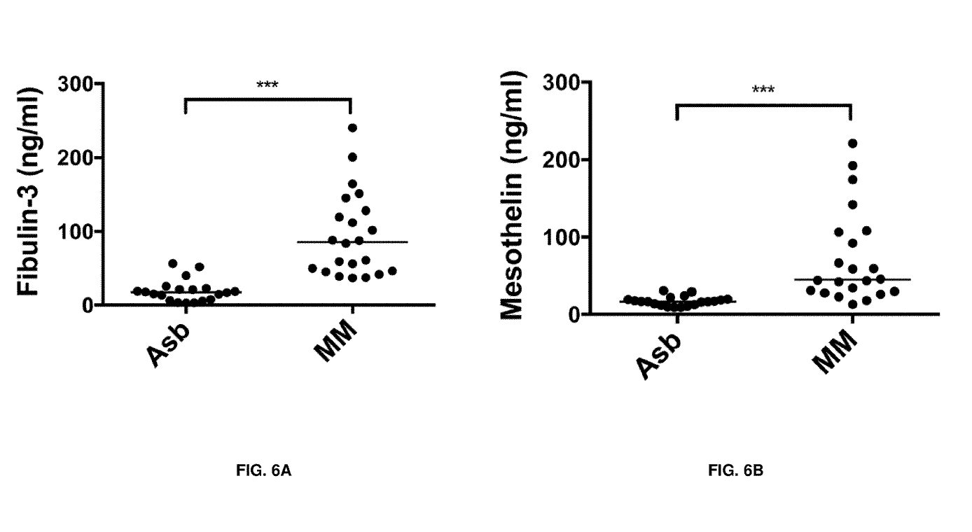

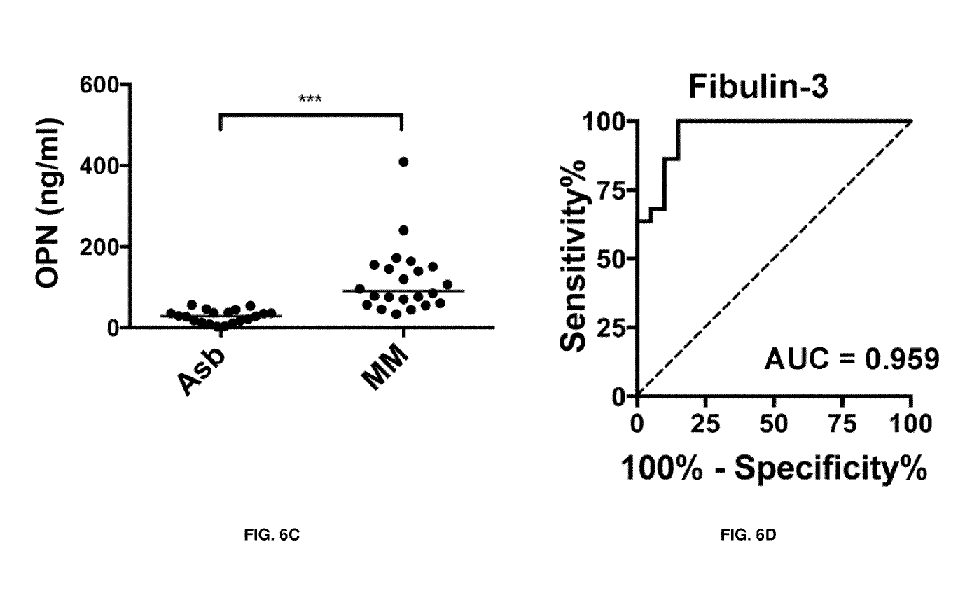

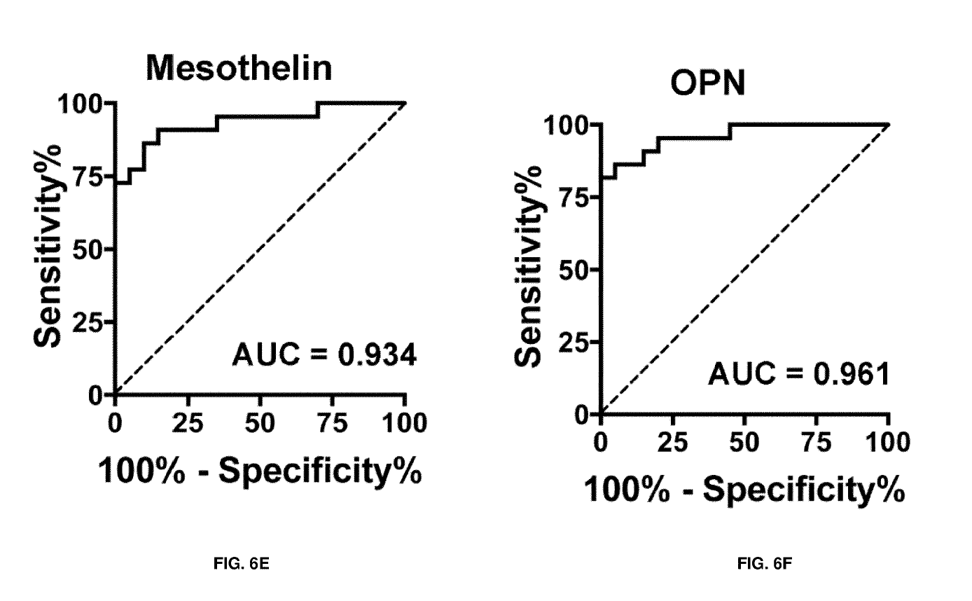

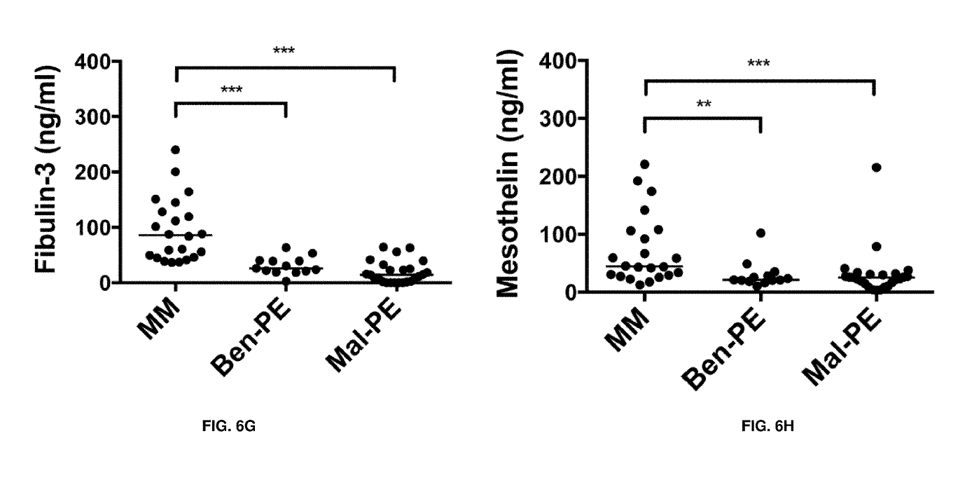

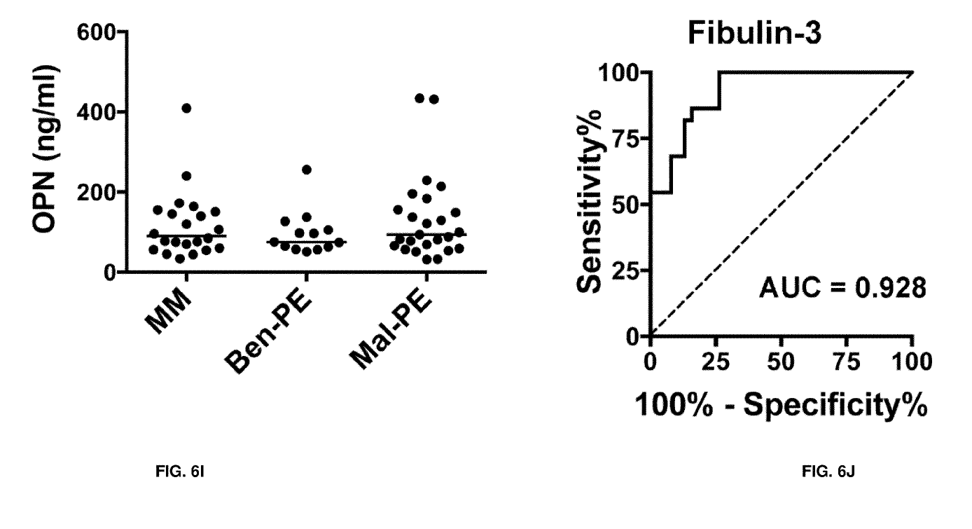

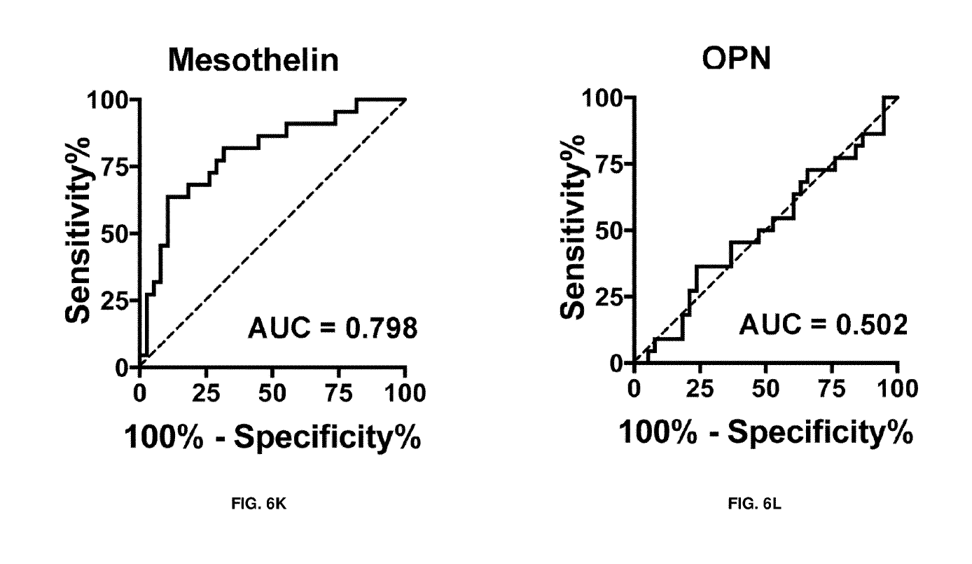

The levels of three additional possible MM biomarkers (fibulin-3, mesothelin, and OPN) from the same asbestos-exposed individuals and MM patients were measured and compared to total and hyper-acetylated HMGB1. All three biomarkers were significantly higher in MM patients than those in the asbestos-exposed individuals (P<0.001) (FIGS. 6A-C, Table 10). Fibulin-3 had AUC of 0.959 (95% CI 0.905-1.000) (FIG. 6D) Mesothelin had AUC of 0.934 (95% CI 0.858-1.000) (FIG. 6E), and OPN had AUC of 0.961 (95% CI 0.910-1.000) (FIG. 6F).

TABLE 10
Levels of additional biomarkers in studied cohorts

| | Fibulin-3, ng/ml | OPN, ng/ml | Mesothelin, ng/ml |
|---|---|---|---|
| | (median, | (median, | (median, |
| | 1^{st} quartile-3^{rd} | 1^{st} quartile-3^{rd} | 1^{st} quartile-3^{rd} |
| | quartile) | quartile) | quartile) |
| Asbestos-exposed | 17.36, 6.91-22.14 | 28.89, 15.12-37.05 | 16.49, 12.08-19.40 |
| MM | 85.73, 46.25-128.07 | 90.20, 60.24-150.93 | 44.92, 29.59-106.31 |
| Benign Pleural | 26.20, 20.13-40.07 | 75.68, 60.20-115.99 | 21.38, 19.76-31.98 |
| Effusion | | | |
| Malignant (non-MM) | 14.63, 1.82-29.10 | 93.71, 62.72-169.86 | 25.56, 12.99-31.78 |
| Pleural Effusion | | | |

The levels of all three biomarkers (fibulin-3, mesothelin, and OPN) were also measured and compared in MM patients to patients with pleural effusions due to other causes. The levels of fibulin-3 were significantly higher in MM patients than those in patients with either benign pleural effusion or malignant non-MM effusion (P<0.001) (FIG. 6G). Mesothelin levels were higher in MM patients than the benign effusion group (P<0.01) or the non-MM malignant effusion group (P<0.001) (FIG. 6H). OPN levels did not show significant difference among the groups (FIG. 6I). Fibulin-3 had an AUC of 0.928 (95% CI 0.868-0.989)(FIG. 6J), mesothelin had an AUC of 0.798 (95% CI 0.678-0.918) (FIG. 6K), and OPN had an AUC of 0.502 (95% CI 0.348-0.657) (FIG. 6L). None of these biomarkers reached the sensitivity and specificity achieved by hyper-acetylated HMGB1 (AUC of 1.000) to discriminate these two cohorts (FIG. 4D). Total HMGB1 (AUC=0.860) and hyper-acetylated HMGB1 (AUC=0.837), performed better than OPN and mesothelin in this comparison (FIGS. 5C-D and 6H-I). Among MM patients, levels of total and hyper-acetylated HMGB1 did not correlate with any of the other biomarkers. Without being limited by any theory, these data suggest independent roles of these molecules in MM.

Accordingly, measurement of hyper-acetylated HMGB1 in accordance with methods and/or kits of some embodiments herein discriminates subjects with MM from subject with pleural effusions with a greater degree of sensitivity and specificity than fibulin-3, mesothelin, and/or OPN.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims. One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method of detecting an HMGB1 isoform signature in a sample of a subject, the method comprising:
providing a sample of a subject;
contacting the sample with at least one of:
(a) an antibody that binds specifically to hyper-acetylated HMGB1;
(b) an antibody that binds specifically to disulfide HMGB1 (HMGB1C23-C45);
(c) an antibody that binds specifically to hypo-acetylated HMGB1;
(d) an antibody that binds specifically to fully reduced HMGB1;
(e) an antibody that binds specifically to hyper-acetylated disulfide HMGB1;
(f) an antibody that binds specifically to hyper-acetylated fully reduced HMGB1;
(g) an antibody that binds specifically to hypo-acetylated disulfide HMGB1; or
(h) an antibody that binds specifically to hypo-acetylated fully-reduced HMGB1; and detecting a level of the antibody bound to HMGB1 in the sample.

2. The method of claim 1, wherein the method is performed in vitro.

3. The method of any one of claims 1-2, further comprising comparing the level of bound antibody to a predetermined level.

4. The method of claim 3, wherein binding of any of (a)-(h) below the predetermined level identifies an HMGB1 isoform signature characteristic of a healthy subject.

5. The method of claim 3, wherein binding of any of (a)-(h) above the predetermined level identifies an HMGB1 isoform characteristic of a subject that has at least one of asbestos exposure, malignant mesothelioma or an inflammatory cancer.

6. The method of claim 3, wherein the sample is contacted with (a) and (c), and wherein binding of (c) above a first predetermined level and binding of (a) above a second predetermined level identifies an HMGB1 isoform characteristic of a subject that has malignant mesothelioma or an inflammatory cancer.

7. The method of claim 3 or claim 6, wherein the sample is contacted with (a) and (c), and wherein binding of (c) above a first predetermined level and binding of (a) below a second predetermined level identifies an HMGB1 isoform characteristic of a subject that has asbestos exposure, but not malignant mesothelioma.

8. The method of claim 3, wherein the sample is contacted with (b) and (d), and wherein binding of (d) above a first predetermined level and binding of (b) above a second predetermined level identifies an HMGB1 isoform characteristic of a subject that has malignant mesothelioma or an inflammatory cancer.

9. The method of claim 3 or claim 6, wherein the sample is contacted with (b) and (d), and wherein binding of (d) above a first predetermined level and binding of (b) below a second predetermined level identifies an HMGB1 isoform characteristic of a subject that has asbestos exposure, but not malignant mesothelioma.

10. The method of claim 3, wherein the sample is contacted with (a) and (c), and wherein binding of (c) above a first predetermined level and binding of (a) above a second predetermined level identifies an HMGB1 isoform characteristic of a subject that has an inflammatory cancer.

11. The method of claim 3, wherein the sample is contacted with (b) and (d), and wherein binding of (d) above a first predetermined level and binding of (b) above a second predetermined level identifies an HMGB1 isoform characteristic of a subject that has an inflammatory cancer.

12. The method of claim 3, wherein binding of any of (a)-(h) above the predetermined level identifies an HMGB1 isoform characteristic of a subject that has an inflammatory cancer.

13. The method of any one of claim 5, 10, 11, or 12, wherein the inflammatory cancer is selected from mesothelioma, colorectal cancer, pancreatic cancer, breast cancer, and liver cancer.

14. The method of any one of claims 1-13, wherein the antibody that binds specifically to hyper-acetylated HMGB1 binds specifically to an acetylated lysine in NLS1 or an acetylated lysine in NLS2 of HMGB1.

15. The method of any one of claims 1-13, wherein the antibody that binds specifically to hyper-acetylated HMGB1 binds specifically to one of acetylated K30, acetylated K43, acetylated K90, or acetylated K141 of HMGB1.

16. The method of any one of claims 1-15, wherein the antibody that binds specifically to disulfide HMGB1 binds specifically to a C23 and/C45 of HMGB1 when C23 and C45 of HMGB1 are joined by a disulfide bond.

17. The method of any one of claims 1-15, wherein the antibody that binds specifically to fully reduced HMGB1 binds specifically to at least one of reduced C23, reduced C45, or reduced C106 of HMGB1.

18. The method of any one of claims 1-17, wherein the sample comprises a serum sample or a plasma sample.

19. The method of any one of claims 1-18, further comprising comparing binding of the antibody to a negative control.

20. The method of claim 19, wherein the negative control comprises a sample of an individual known not to have mesothelioma.

21. The method of any one of claims 1-19, further comprising:
contacting the sample with an antibody that binds specifically to HMGB1 of any isoform;
determining the level of total HMGB1 in the sample; and
comparing the level of total HMGB1 to a negative control or predetermined level.

22. The method of claim 21, wherein if the subject is a smoker, the negative control is a smoker, and wherein if the subject comprises a non-smoker, the negative control comprises a non-smoker.

23. The method of claim 21, wherein if the subject is a smoker, the predetermined level is higher than if the subject is a non-smoker.

24. A method of identifying malignant mesothelioma in a subject, the method comprising:
providing a sample of the subject;
contacting the sample with at least one of:
(a) an antibody that binds specifically to hyper-acetylated HMGB1;
(b) an antibody that binds specifically to disulfide HMGB1 (HMGB1C23-C45);
(c) an antibody that binds specifically to hypo-acetylated HMGB1; or
(d) an antibody that binds specifically to fully reduced HMGB1;
detecting a level of the antibody bound to HMGB1 in the sample;
comparing the level of binding of antibody to a predetermined level; and
diagnosing the subject as having malignant mesothelioma when the level of antibody bound to HMGB1 is greater than the predetermined level.

25. The method of claim 24 wherein the sample is contacted with (a), and wherein the predetermined level is a level of the antibody of (a) bound to 1 ng/mL hyper-acetylated HMGB1.

26. The method of claim 24 wherein the sample is contacted with (a), and wherein the predetermined level is a level of the antibody of (a) bound to 5 ng/mL hyper-acetylated HMGB1.

27. The method of claim 24 wherein the sample is contacted with (b), and wherein the predetermined level is a level of the antibody of (b) bound to 0.1 ng/mL disulfide HMGB1.

28. The method of claim 24 wherein the sample is contacted with (b), and wherein the predetermined level is a lower limit of detection of the antibody of (b) bound to disulfide HMGB1.

29. The method of claim 24 wherein the sample is contacted with (b), and wherein the predetermined level is a level of the antibody of (b) bound to 5 ng/mL disulfide HMGB1.

30. The method of any one of claims 24-29, comprising contacting the sample with at least two of (a), (b), (c), and (d).

31. The method of claim 24 wherein the sample is contacted with (a), and wherein the predetermined level is a level of the antibody of (a) bound to 1 ng/mL hyper-acetylated HMGB1, and wherein the sample is further contacted with (b), (c), or (d).

32. The method of claim 24 wherein the sample is contacted with (b), and wherein the predetermined level is a lower limit of detection of the antibody of (b) bound to disulfide HMGB1, and wherein the sample is further contacted with (a), (c), or (d).

33. The method of claim 24 wherein the sample is contacted with (b), and wherein the predetermined level is a level of the antibody of (b) bound to 0.1 ng/ml disulfide HMGB1, and wherein the sample is further contacted with (a), (c), or (d).

34. The method of any one of claims 24-33, wherein the biological sample of the subject comprises a fluid.

35. The method of claims 24-33, wherein the sample of the subject comprises a serum or a plasma.

36. The method of any one of claims 24-35, wherein the antibody that binds specifically to hyper-acetylated HMGB1 binds specifically to an acetylated lysine in NLS1 or an acetylated lysine in NLS2 of HMGB1.

37. The method of any one of claims 24-35, wherein the antibody that binds specifically to hyper-acetylated HMGB1 binds specifically to one of acetylated K30, acetylated K43, acetylated K90, or acetylated K141 of HMGB1.

38. The method of any one of claims 24-37, wherein the antibody that binds specifically to disulfide HMGB1 binds specifically to a C23 and/C45 of HMGB1 when C23 and C45 of HMGB1 are joined by a disulfide bond.

39. The method of any one of claims 24-37, wherein the antibody that binds specifically to fully reduced HMGB1 binds specifically to at least one of reduced C23, reduced C45, or reduced C106 of HMGB1.

40. The method of any one of claims 24-39, wherein the sample is contacted with (a), and wherein the predetermined level is a level of the antibody of (a) bound to 2 ng/mL hyper-acetylated HMGB1.

41. The method of any one of claims 24-39, wherein the sample is contacted with (a), and wherein the predetermined level is a level of the antibody of (a) bound to 2 ng/mL hyper-acetylated HMGB1, and wherein the sample is further contacted with (b), (c), or (d).

42. The method of any of claims 1-41, wherein the antibody further comprises a detectable moiety.

43. The method of any of claims 1-42, wherein the method comprises an in vitro method.

44. The method of any of claims 1-42, wherein the method comprises an in vivo method.

45. A method of differentiating between malignant mesothelioma and asbestos-exposure in a subject, the method comprising:
providing a sample from a subject suspected of having malignant mesothelioma;
contacting the sample with at least one of:
(a) an antibody that binds specifically to hyper-acetylated HMGB1;
(b) an antibody that binds specifically to disulfide HMGB1 (HMGB1C23-C45); or
(c) an antibody that binds specifically to fully reduced HMGB1; and
detecting a level of the antibody bound to hyper-acetylated HMGB1, disulfide HMGB1 (HMGB1C23-C45) or fully reduced HMGB1; and
comparing the level of antibody bound to hyper-acetylated HMGB1, disulfide HMGB1, or fully reduced HMGB1 to a predetermined level, wherein a level of hyper-acetylated HMGB1, disulfide HMGB1, or fully reduced HMGB1 greater than a predetermined level indicates a presence of malignant mesothelioma in the subject.

46. The method of claim 45, wherein the sample is contacted with (a), and wherein the predetermined level is a level of the antibody of (a) bound to 2 ng/mL of hyper-acetylated HMGB1.

47. The method of claim 45, wherein the sample is contacted with (a), and wherein the predetermined level is a level of the antibody of (a) bound to 5 ng/mL of hyper-acetylated HMGB1.

48. The method of any one of claims 45-47, wherein the sample is contacted with (b), and wherein the predetermined level is a lower limit detection of the antibody of (b).

49. The method of any one of claim 45-47, wherein the sample is contacted with (b), and wherein the predetermined level is a 5-fold increase over a lower limit detection of the antibody of (b).

50. The method of any one of claims 45-47, wherein the sample is contacted with (b), and wherein the predetermined level is a level of the antibody of (b) bound to 5 ng/mL of disulfide HMGB1.

51. The method of any one of claims 45-50, wherein the antibody that binds specifically to hyper-acetylated HMGB1 binds specifically to an acetylated lysine in NLS1 or an acetylated lysine in NLS2 of HMGB1.

52. The method of any one of claims 45-50, wherein the antibody that binds specifically to hyper-acetylated HMGB1 binds specifically to one of acetylated K30, acetylated K43, acetylated K90, or acetylated K141 of HMGB1.

53. The method of any one of claims 45-52, wherein the antibody that binds specifically to disulfide HMGB1 binds specifically to a C23 and/C45 of HMGB1 when C23 and C45 of HMGB1 are joined by a disulfide bond.

54. The method of any one of claims 45-53, wherein the antibody that binds specifically to fully reduced HMGB1 binds specifically to at least one of reduced C23, reduced C45, or reduced C106 of HMGB1.

55. The method of any one of claims 45-54, wherein the method comprises an in vitro method.

56. The method of any one of claims 45-54, wherein the method comprises an in vivo method.

57. The method of any one of claims 45-56, wherein the antibody comprises a detectable moiety.

58. A method of identifying asbestos exposure, malignant mesothelioma, or inflammatory cancer in a subject, the method comprising:
determining an amount of an isoform of HMGB1 in a sample of the subject, wherein the isoform of HMGB1 is selected from:
(a) a 24.585 KDa isoform of HMGB1;
(b) a 24.587 KDa isoform of HMGB1;
(c) a 25.467 KDa isoform of HMGB1; or
(d) a 25.469 KDa isoform of HMGB1; and
comparing an amount of the isoform of HMGB1 to a predetermined level, wherein an amount greater than the predetermined level identifies asbestos exposure, malignant mesothelioma, or inflammatory cancer in the subject.

59. The method of claim 58, wherein a level of (c) or (d) greater than 5 ng/mL indicates a presence of mesothelioma, and wherein a level of (a) or (d) greater than 5-fold of a lower limit of detection indicates a presence of mesothelioma or inflammatory cancer.

60. The method of claim 58, wherein a level of (c) or (d) less than 1 ng/mL and a level of (a) or (d) greater than 2 ng/mL indicates asbestos exposure, but not mesothelioma.

61. The method of claim 58 or 59, wherein the inflammatory cancer is selected from: mesothelioma, colorectal cancer, pancreatic cancer, breast cancer, and liver cancer.

62. The method of any one of claims 58-61, wherein the amount of the isoform of HMGB1 in the sample is determined by mass spectrometry.

63. The method of any one of claims 58-61, wherein the amount of the isoform of HMGB1 in the sample is determined by contacting the sample with:
(a) an antibody that binds specifically to hypo-acetylated disulfide HMGB1 to detect the 24.585 kDa isoform;
(b) an antibody that binds specifically to hypo-acetylated fully reduced HMGB1 to detect the 24.587 kDa isoform;
(c) an antibody that binds specifically to hyper-acetylated disulfide HMGB1 to detect the 25.467 isoform; or
(d) an antibody that binds specifically to hyper-acetylated fully-reduced HMGB1 to detect the 25.693 kDa isoform.

64. A immunoassay kit comprising at least one of:
(a) a first antibody that binds specifically to hyper-acetylated HMGB1;
(b) a first antibody that binds specifically to disulfide HMGB1 (HMGB1C23-C45);
(c) a first antibody that binds specifically to hypo-acetylated HMGB1;
(d) a first antibody that binds specifically to fully reduced HMGB1;
(e) a first antibody that binds specifically to hyper-acetylated disulfide HMGB1;
(f) a first antibody that binds specifically to hyper-acetylated fully reduced HMGB1;
(g) a first antibody that binds specifically to hypo-acetylated disulfide HMGB1; or
(h) a first antibody that binds specifically to hypo-acetylated fully-reduced HMGB1; and
a first detectable moiety.

65. The immunoassay kit of claim 64, wherein the immunoassay comprises an ELISA.

66. The immunoassay kit of claim 64, wherein the immunoassay comprises a lateral flow assay.

67. The immunoassay kit of any one of claims 64-66, wherein the first antibody that binds specifically to hyper-acetylated HMGB1 binds specifically to an acetylated lysine in NLS1 or an acetylated lysine in NLS2 of HMGB1.

68. The immunoassay kit of any one of claims 64-66, wherein the first antibody that binds specifically to hyper-acetylated HMGB1 binds specifically to one of acetylated K30, acetylated K43, acetylated K90, or acetylated K141 of HMGB1.

69. The immunoassay kit of any one of claims 64-66, wherein the first antibody that binds specifically to disulfide HMGB1 binds specifically to a C23 and/C45 of HMGB1 when C23 and C45 of HMGB1 are joined by a disulfide bond.

70. The immunoassay kit of any one of claims 64-66, wherein the first antibody that binds specifically to fully reduced HMGB1 binds specifically to at least one of reduced C23, reduced C45, or reduced C106 of HMGB1.

71. The immunoassay kit of any one of claims 64-66, further comprising a second antibody that binds specifically to HMGB1.

72. The immunoassay kit of claim 71, wherein the second antibody is immobilized on a substrate.

73. The immunoassay kit of claim 71, wherein the first antibody is immobilized on a substrate.

74. The immunoassay kit of claim 71 or 73, wherein the second antibody comprises the detectable moiety.

75. The immunoassay kit of claim 71 or 73, further comprising a third antibody that binds specifically to the second antibody, wherein the third antibody comprises the detectable moiety.

76. The immunoassay kit of any one of claims 64-75, wherein the first antibody comprises the detectable moiety.

77. The immunoassay kit of claim 65 or 66, further comprising a third antibody that binds specifically to the first antibody, wherein the third antibody comprises the detectable moiety.

78. The immunoassay kit of any one of claims 64-77, wherein the first antibody comprises a monoclonal antibody.

79. The immunoassay kit of claim 64, wherein the immunoassay comprises no wash assay.

80. The immunoassay kit of claim 79, further comprising a second detectable moiety, wherein the first detectable moiety and the second detectable moiety comprise a FRET pair.

81. The method of any one of claims 24-44, further comprising contacting the sample with an antibody that binds specifically to total HMGB1.

82. The method of any one of claim 3-20, 20, 21, or 24-44, wherein the predetermined level of is selected based on at least one of sex, age, or smoking status of the subject.

83. The method of any one of claim 3-20, 24-44, 81, or 82, wherein the predetermined level is higher for a female subject than for a male subject.

84. The method of any one of claim 3-20, 24-44, 81, or 82, wherein the predetermined level is higher for a subject 55 years or older than for a subject under the age of 55 years.

85. The method of any one of claim 3-20, 24-44, 81, or 82, wherein the predetermined level is higher for a subject who is a previous or active smoker than for a subject who is a non-smoker.

86. The method of any one of claim 1-57, 63, or 81-85, wherein the antibody comprises a non-native antibody.

87. The method of any one of claim 1-57, 63, or 81-85, wherein the antibody comprises a monoclonal antibody.

88. The method of any one of claim 1-57, 63, or 81-85, wherein the antibody comprises an antibody engineered against an HMGB1 isoform.

89. The method of any one of claim 1-57, 63, or 81-88, further comprising detecting a binding pattern of the antibody.

90. The method of any one of claim 1-57, 63, or 81-89, wherein the predetermined level is based upon an electronically stored or written value.

91. The method of any one of claim 1-57, 63, or 81-89, wherein the predetermined level is based upon a control or standard sample.

92. The method of any one of claim 1-57, 63, or 81-91, further comprising recommending a treatment regimen for malignant mesothelioma or inflammatory cancer for a subject for whom the level of bound antibody exceeds the predetermined level.

93. The method of any one of claim 1-57, 63, or 81-91, further comprising recommending a treatment regimen for malignant mesothelioma for a subject for whom the level of bound antibody exceeds the predetermined level.

94. The immunoassay kit of any one of claims 64-80, wherein the antibody comprises a non-native antibody.

95. The immunoassay kit of any one of claims 64-80, wherein the antibody comprises a monoclonal antibody.

96. The immunoassay kit of any one of claims 64-80, wherein the antibody was engineered against an HMGB1 isoform.

97. A kit for detecting at least on HMGB1 isoform by mass spectrometry, the kit comprising:
a first standard consisting essentially of a single isoform of HMGB1, wherein the single isoform is selected from:
(a) a 24.585 KDa isoform of HMGB1;
(b) a 24.587 KDa isoform of HMGB1;
(c) a 25.467 KDa isoform of HMGB1; and
(d) a 25.469 KDa isoform of HMGB1.

98. The kit of claim 97, further comprising a second standard consisting essentially of a single isoform of HMGB1, wherein the single isoform is selected from:
(a) a 24.585 KDa isoform of HMGB1;
(b) a 24.587 KDa isoform of HMGB1;
(c) a 25.467 KDa isoform of HMGB1; and
(d) a 25.469 KDa isoform of HMGB1,
wherein the second standard consists essentially of a different HMGB1 isoform than the first standard.

99. The kit of claim 97 or 98, further comprising an antibody that binds specifically to HMGB1.

100. The method of claim 3, wherein binding of any of (a)-(h) below the predetermined level identifies an HMGB1 isoform signature characteristic of a subject who does not have malignant mesothelioma.

101. The method of claim 24, further comprising diagnosing the subject as not having malignant mesothelioma, when the level of antibody bound to HMGB1 is less than the predetermined level.


SEQUENCE LISTING (1 sequence)

SEQ ID NO: 1 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE